(19) United States
(12) Patent Application Publication
(10) Pub. No.: US 2017/0050173 A1
(43) Pub. Date: Feb. 23, 2017

(54) PROGRAMMABLE MIP CATCH AND RELEASE TECHNOLOGY

(71) Applicant: The Decaf Company, LLC, Danville, CA (US)

(72) Inventors: James P. Farr, Dublin, CA (US); William P. Sibert, Danville, CA (US); Michael J. Petrin, Pleasant Hill, CA (US); Marion M. Stuckey, Danville, CA (US)

(73) Assignee: The Decaf Company, LLC, Danville, CA (US)

(21) Appl. No.: 15/237,507

(22) Filed: Aug. 15, 2016

Related U.S. Application Data
(60) Provisional application No. 62/207,231, filed on Aug. 19, 2015.

(51) Int. Cl. B01J 20/26; A61K 31/522; A61K 47/32; C08F 20/06

(52) U.S. Cl. CPC B01J 20/268; C08F 20/06; A61K 31/522; A61K 47/32

(57) ABSTRACT

Programmable molecular imprinted polymers (MIPs) that have modified binding site kinetics for target imprintable entities (TIEs) that operate to control the adsorption, binding, release and equilibrium distribution of related materials into and out of the MIPs, which are useful for the controlled adsorption, controlled release and control of concentrations of such materials in media including gases, liquids, fluids, biological systems, solutions and other environments. When a collective plurality of the MIPs with modified binding site kinetics are combined, the resulting MIP systems can be tailored to exhibit pseudo zero- and first-order kinetics, as well as higher kinetic profiles, and when further combined with time-delay functionality, can be tailored to exhibit delayed uptake and release, ramped uptake and release of materials, step functions, polynomial, geometric, exponential and other unique kinetic profiles of material exchange between the novel MIPs and a fluid media that are not readily achievable by other means.

### PRIORITY

This application claims the benefit of co-pending provisional patent application No. 62/207,231, entitled PROGRAMMABLE MIP CATCH AND RELEASE TECHNOLOGY, filed by the same inventors on Aug. 19, 2016 which is incorporated by reference, together with its appendix, as if fully set forth herein.

### INTRODUCTION

The present disclosure relates generally to programmable molecular imprinted polymers (MIPs) that have modified binding site kinetics for target imprintable entities (TIEs) and that operate to control the adsorption, binding, release and transit of materials into and out of the MIPs matrices, which are useful for the controlled adsorption, release and control of concentrations of materials in fluid media, biological systems, gases, liquids, solutions and other environments. When a collective plurality of the novel MIPs with modified binding site kinetics are combined, the resulting MIP systems surprisingly can be tailored to exhibit pseudo zero- and first-order kinetics, as well as higher order behaviors, and when further combined with time-delay functionality, can be tailored to exhibit delayed uptake and release, ramped uptake and release of materials, step functions, polynomial, geometric, exponential and other unique kinetic profiles of material exchange between the novel MIPs and a fluid media that are not readily achievable by any other means.

### SUMMARY

One aspect of the present disclosure is a polymeric matrix comprising a plurality of binding sites within a molecularly imprinted polymer (MIP) that exhibit at least one average associative binding constant (kₘ) with respect to a selected material (m); wherein the magnitude of said average associative binding constant is significantly different than that of the average equilibrium associative binding constant exhibited by said polymer matrix for a reference target imprinted entity (TIE) used as the template forming entity in the formation of said plurality of binding sites within said MIP; and wherein said plurality of binding sites operate to enable the controlled capture (adsorption) and the controlled release (de-adsorption) of said selected material, and combinations thereof, when in contact with a fluid media.

Another aspect of the present disclosure is a polymeric matrix formed by means of polymerizing a plurality of monomers into a three dimensional matrix in the presence of a target imprinted entity, a porogen, a solvent, optionally a cosolvent, optionally comonomers, optionally a pore modifying agent, and optionally a cross-linking agent, and combinations thereof; wherein the polymeric matrix exhibits at least one average associative binding constant (kₘ) with respect to a selected material (m); wherein the magnitude of said average associative binding constant is significantly different than that of the average equilibrium associative binding constant exhibited by said polymer matrix for a reference target imprinted entity (TIE).

Another aspect of the present disclosure is a polymeric matrix having a plurality of binding sites that exhibit at least one average associative binding constant (km) that is suboptimal with respect to a selected material (m) compared to the average associative binding constant (k_{TIE}) of said polymer matrix for a target imprinted entity (TIE) used as the template forming entity in the formation of said plurality of binding sites within said molecularly imprinted polymer.

A further aspect of the present disclosure is a polymeric matrix having two or more sets of binding sites wherein each said set of binding sites exhibits a significantly different average associative binding constant (kmₙ, n=1,2,3 . . . ) with respect to a selected material; wherein at least two of said sets (n) of binding sites are formed during a polymerization process using at least one second polymerization aid that is different from a first polymerization aid employed in the formation of a first set of binding sites; wherein said second polymerization aid is selected from a different TIE, a different porogen, a different solvent, a different cosolvent, a different pore modifying agent, or combinations thereof; and wherein said significantly different average associative binding constants differ by at least one least significant difference (LSD) unit at the 80% confidence level.

Another aspect of the present disclosure is a polymeric matrix of claim having a set of binding sites that exhibit an average associative binding constant that is significantly lower than that of the average equilibrium associative binding constant exhibited by said polymer matrix for a target imprinted entity (TIE) used as the template forming entity in the formation of said plurality of binding sites within said MIP; wherein each of said average equilibrium associative binding constants for each of said sets of binding constants are each significantly different in magnitude from each other; and wherein said average equilibrium associative binding constants differ by at least on least significant difference (LSD) unit at the 80% confidence level, or alternatively at the 90% confidence level, or alternatively at the 95% confidence level, or alternatively at the 99% confidence level.

Another aspect of the present disclosure is the use of the novel polymeric matrices to control the catching and release of a material between the molecularly imprinted polymers and a fluid media selected from air, an aqueous solution, a bodily fluid, a liquid, a chemical composition, a solvent, a vapor, water, and combinations thereof.

Yet a further aspect of the present disclosure is a polymeric matrix operating to controllably release a selected material comprising a molecularly imprinted polymer templated using a target imprinted entity that differs from said selected material in at least one feature selected from a chemical, physical or stereo isometric characteristic of said selected material.

A further aspect of the present disclosure is a polymeric matrix operating to controllably catch and/or release a selected material comprising a molecularly imprinted polymer templated using a target imprinted entity that shares at least one common attribute with said selected material; wherein said at least one common attribute is selected from an atom, a chemical group, a chemical bond, a substituent group, an atomic arrangement, a molecular arrangement, a chemical structure, a charge bearing chemical group, an isomer, a stereo-isomer, a sequence of atomic or molecular entities, a three-dimensional structure or portion of a three-dimensional structure, and combinations thereof.

Another aspect of the present disclosure is the use of a time-delay element associated with at least one of the novel molecularly imprinted polymers or matrices.

One additional aspect of the present disclosure is a polymeric matrix comprising a combination of two or more distinct molecularly imprinted polymer matrices each having at least one or a plurality of sets of binding sites wherein each said set of binding sites exhibits an average associative binding constant (km) with respect to said selected material; wherein each of said sets (n) of binding sites is formed during a polymerization process using one of a different monomer, a different comonomer, a different polymer, a different cross-linking agent, a different TIE, a different porogen, a different solvent, a different cosolvent, a different pore modifying agent, or combinations thereof.

Yet a further aspect of the present disclosure is a polymer matrix further comprising one or a plurality of distinct time-delay elements each associated with one or more of said distinct molecularly imprinted polymer matrices each having a time delay factor or dissolution characteristic that is significantly different from each other of said other time delay factors or dissolution characteristics.

Another aspect of the present disclosure is a polymer matrix wherein at least one average associative binding constant (kₘ) has a value that is less than the average associative binding constant for the TIE used to template said molecular imprinted polymer by at least one least significant difference (LSD) unit at an 80% confidence level, or alternatively at the 90% confidence level, or alternatively at the 95% confidence level, or alternatively at the 99% confidence level.

A further aspect of the present disclosure is a polymer matrix having a set of average associative binding constants each having values that are less than the average associative binding constant for the TIE used to template said molecular imprinted polymer, and wherein each of said plurality of average associative binding constants for said material are significantly different from each other by at least one least significant difference (LSD) unit at an 80% confidence level, or alternatively at the 90% confidence level, or alternatively at the 95% confidence level, or alternatively at the 99% confidence level.

Yet another aspect of the present disclosure is a polymer matrix having a set of average associative binding constants each having values that are less than the average associative binding constant for the TIE used to template said molecular imprinted polymer; wherein each of said plurality of average associative binding constants for said material differ by at least a factor of two in magnitude with respect to each other.

One aspect of the present disclosure is a polymer matrix having a set of average associative binding constants each having values that are less than the average associative binding constant for the TIE used to template said molecular imprinted polymer; wherein at least two of said plurality of average associative binding constants for said material differ by at least a factor of two in magnitude with respect to each other.

A further related aspect of the present disclosure is a polymer matrix having a set of average associative binding constants each having values that are significantly less than the average associative binding constant for the TIE used to template said molecular imprinted polymer; wherein at least two of said plurality of average associative binding constants for said material differ by at least a factor of ten in magnitude from each other.

One aspect of the present disclosure is a molecularly imprinted polymer comprising a polymeric matrix formed in the presence of a target imprintable entity, a plurality of monomers, a solvent, optionally one or more porogens, and optionally a second plurality of comonomers; wherein said polymeric matrix exhibits at least one set of suboptimal binding sites with an average associative binding constant for a reference material that is lower in magnitude with respect to the average associative binding constant exhibited by the target imprintable entity employed; wherein said reference material is selected from the group consisting of said target imprintable entity, an analog, isomer or derivative of said target imprintable entity, an associative molecule, and combinations thereof.

Yet another aspect of the present disclosure is a molecularly imprinted polymer comprising a polymeric matrix formed in the presence of a target imprintable entity, a plurality of monomers, at least one porogen, a solvent, and optionally additional comonomers, copolymers, cross-linking agents, coupling agents, and combinations thereof; wherein said polymeric matrix exhibits a plurality of suboptimal binding sites with an average associative binding constant for a reference material that is lower in magnitude with respect to the average associative binding constant exhibited by the target imprintable entity employed; wherein said reference material is selected from the group consisting of said target imprintable entity, an analog, isomer or derivative of said target imprintable entity, an associative molecule, and combinations thereof.

A further aspect of the present disclosure is a method of controlling the concentration of a material within a fluid media comprising the use of a polymeric matrix comprising: a plurality of binding sites within a molecularly imprinted polymer (MIP) that exhibit at least one average associative binding constant (kₘ) with respect to a selected material (m); wherein the magnitude of said average associative binding constant is significantly different than that of the average equilibrium associative binding constant exhibited by said polymer matrix for a reference target imprinted entity (TIE) used as the template forming entity in the formation of said plurality of binding sites within said MIP; and wherein said plurality of binding sites operate to enable the controlled capture and the controlled release of said selected material, and combinations thereof, when in contact with a fluid media.

Yet a further aspect of the present disclosure is a method of controlling the concentration of a material within a fluid media comprising the use of a polymeric matrix comprising: two or more sets of binding sites; wherein each said set of binding sites exhibits a significantly different average associative binding constant (kmₙ, n=1,2,3 . . . ) with respect to said selected material; wherein at least two of said sets (n) of binding sites are formed during a polymerization process using at least one second polymerization aid than is different than a first polymerization aid employed in the formation of a first set of binding sites; wherein said second polymerization aid is selected from a different TIE, a different porogen, a different solvent, a different cosolvent, a different pore modifying agent, or combinations thereof; and wherein said significantly different average associative binding constants differ by at least on least significant difference (LSD) unit at the 80% confidence level.

Yet another related aspect of the present disclosure is a method further employing a second polymer matrix; wherein said second polymer matrix comprises one or a plurality of distinct delay elements each associated with a first or second molecularly imprinted polymer; wherein said delay element is selected from: time release coating, each having a time delay factor or dissolution characteristic that is significantly different from each other of said other time delay factors or dissolution characteristics.

An additional aspect of the present disclosure is a molecularly imprinted polymer system for use in the catch and/or release of multiple materials comprising: (a) a first molecularly imprinted polymer with at least one suboptimal average associative binding constant with respect to a first material to be released; (b) a second molecularly imprinted polymer with at least one suboptimal average associative binding constant with respect to a second material to be captured; wherein said first molecularly imprinted polymer is dosed with said first material to a desired degree of saturation; wherein said first molecularly imprinted polymer and said second molecularly imprinted polymer are introduced or contacted with a fluid media; and wherein said first and said second molecularly imprinted polymers operate to controllably release a first material into said fluid media and controllably adsorb a second material from said fluid media, respectively.

One further aspect of the present disclosure is a molecularly imprinted polymer system for use in the controlled release of a selected material comprising: (a) a first molecularly imprinted polymer with at least one first suboptimal average associative binding constant with respect to said selected material; (b) a second molecularly imprinted polymer with at least one second suboptimal average associative binding constant with respect to said selected material; wherein said second suboptimal average associative binding constant differs in magnitude from said first suboptimal average associative binding constant by at least one least significant difference (LSD) at an 80% confidence level; wherein said first molecularly imprinted polymer is dosed with said first material to a desired degree of saturation; wherein said first molecularly imprinted polymer and said second molecularly imprinted polymer are introduced or contacted with a fluid media so as to be in fluidic communication with each other; and wherein said first and said second molecularly imprinted polymers operate to controllably release said selected material into said fluid media following a desired release profile corresponding to the a release rate proportional to the ratio of said first and said second suboptimal average associative binding constants.

Yet another aspect of the present disclosure is a molecularly imprinted polymer system for use in the controlled release of a selected material comprising: (a) a plurality of molecularly imprinted polymers each exhibiting at least one suboptimal average associative binding constant with respect to said selected material; wherein said suboptimal average associative binding constants of said plurality of molecularly imprinted polymers each exhibit values that differ in magnitude from each other by at least one least significant difference (LSD) at an 80% confidence level; wherein said plurality of molecularly imprinted polymer is dosed with said selected material to a desired degree of saturation; wherein said plurality of molecularly imprinted polymers are introduced or contacted with a fluid media so as to be in fluidic communication with each other; and wherein said plurality of molecularly imprinted polymers operate to controllably release said selected material into said fluid media following a desired release profile corresponding to a profile selected from: pseudo-zero order, pseudo-first order, pseudo-n order, exponential, linear, geometric, polynomial, sigmoidal, and combinations thereof.

In a further related aspect of the present disclosure is a molecularly imprinted polymer system further comprising a time-delay element associated with at least of one of said plurality of molecularly imprinted polymers; wherein said time delay element operates to delay the time of contact between said associated molecularly imprinted polymer and the fluid media in contact therewith for a selected time period determined by said time delay element; wherein said time-delay element is selected from any suitable material that is slowly or sparingly soluble and/or disintegrates over a desired time period within said fluid media so as to require a desired period of time to be sufficiently dissolved or compromised so as to expose the associated molecularly imprinted polymer to said fluid media.

On additional aspect of the present disclosure is a molecularly imprinted polymer system for use in the treatment of a specific biological pathogen, comprising: (a) a first molecularly imprinted polymer matrix templated with at least one molecular recognition pattern corresponding to a surface borne molecular entity associated with the exterior cellular membrane of a specific biological pathogen and that operates to bind said pathogen upon contact; (b) a second molecularly imprinted polymer matrix with at least one suboptimum associative binding constant with respect to a treatment agent effective against said biological pathogen; wherein said second molecularly imprinted polymer matrix is preloaded with said treatment agent after formation and extraction of a suitable templating material; (c) optionally, a time-delay coating around said second MIP matrix bearing said preloaded treatment agent; wherein said coating is effective in shielding said second molecularly imprinted polymer matrix for a desired time period; wherein said second molecularly imprinted polymer matrix with said at least one suboptimal associative binding constant operates to controllably release the preloaded treatment agent at a controlled rate into a fluid media.

Yet another aspect of the present disclosure is a molecularly imprinted polymer system further comprising a third molecularly imprinted polymer matrix; wherein said third molecularly imprinted polymer matrix has been templated with the treatment agent to exhibit a higher associative binding constant than that of said second molecularly imprinted polymer matrix and operates to adsorb excess treatment agent from said surrounding fluid media.

A further aspect of the present disclosure is a molecularly imprinted polymer system further comprising a second delay-release coating around said third molecularly imprinted polymer matrix; wherein said coating is effective in shielding said third molecularly imprinted polymer matrix for a desired second time period that is greater than or equal to the time period exhibited by said delay-release coating around said second molecularly imprinted polymer matrix.

Another aspect of the present disclosure is the combination of these novel molecularly imprinted polymer matrices with one or a plurality of tethering elements that operate to bind the novel polymer matrices to each other or to a target delivery site; wherein said tethering element is selected from a physical link, a chemical bond, a molecular bond, a molecular linker group, a polymer chain, an ionic bond, a physical linker moiety, and/or combinations thereof.

A further related aspect of the present disclosure is the use of the novel molecularly imprinted polymers with a physical linker moiety having at least two or more template groups (T) and at least one spacer group (S); wherein said template group is any molecule or molecular fragment capable of being used as a target imprinted entity (TIE) in the formation of a molecularly imprinted polymer matrix; and wherein said spacer group is any molecule or molecular fragment in the form of a linear chain, branched chain, substituted chain, star polymer, dendritic or any suitable repeating chemical unit; wherein said physical linker moiety has the following structure:

T-(S)n-T

wherein n includes any integer value from n=1 to about 1000 and wherein said template groups operate to bind to a molecularly imprinted polymer that has been imprinted with a target imprinted entity comprising a template group, a chemically modified template group, a molecular analog to said template group bearing at least one common molecular recognition site, and combinations thereof.

Yet another aspect of the present disclosure is a reversible molecularly imprinted polymer association complex comprising: (a) a physical linker moiety comprising a molecule having at least two or more template groups (T) and at least one spacer group (S); wherein said template group is any molecule or molecular fragment capable of being used as a target imprinted entity (TIE) in the formation of a molecularly imprinted polymer matrix; and wherein said spacer group is any molecule or molecular fragment that can be formed into a linear chain, branched chain, or any suitable repeating chemical unit; wherein said physical linker moiety has the following structure:

T-(S)n-T

wherein n includes any integer value from n=1 to about 1000; wherein said template group operates to bind to a molecularly imprinted polymer (MIP) that has been imprinted with a target imprinted entity selected from the group consisting of an unmodified template molecule, a chemically modified template group, a molecular analog to said template group bearing at least one common molecular group or constituent, and combinations thereof; (b) at least two molecularly imprinted polymer matrices each bearing a plurality of surface sites capable of binding to one or more of said template groups of said physical linker moiety; wherein each of said molecularly imprinted matrices each binds to at least one of said template groups of said physical linker moiety to form said molecularly imprinted polymer association complex; wherein said molecularly imprinted polymer association complex has the following general structure:

MIP_{Template}:T-(S)n-T:MIP_{Template}

wherein said association complex is formed by combining the materials (a) and (b) under conditions such that a first template group on a first end of said physical linker moiety binds to a first of said molecularly imprinted polymer matrices; and a second template group on the second end of said same physical linker moiety binds to a second of said molecularly imprinted polymer matrices; wherein said first template group and said second template group are optionally selected from the group consisting of: the same group, a different group, and combinations thereof.

Yet a further related aspect of the present disclosure is the a reversible molecularly imprinted polymer association complex wherein said physical linker moiety comprises a structure:

(T)n-(P)m

wherein, unless otherwise stated, n is an integer from 2 to about 10,000,000 and m is an integer from 1 to about 100,000,000; and wherein P is a polymer selected from a linear, branched or substituted polymer with n number of T substituents and m number of repeated monomers; a star polymer with n number of T substituents and wherein m=1; a dendritic polymer with n number of T substituents located at terminal positions and m=1 to about 1,000; a block copolymer with n number of T substituents and wherein m is the total number of monomer groups of all kinds, copolymers thereof; and combinations thereof.

An additional aspect of the present disclosure is a method of constructing a molecularly imprinted polymer system for the programmed catch and/or release of a selected material comprising: (a) selecting a first molecularly imprinted polymer that feature a first set of binding sites that exhibit a first average associative binding constant with respect to said selected material; (b) selecting a second molecularly imprinted polymer that feature a second set of binding sites that exhibit a second average associative binding constant with respect to said selected material; wherein said first and said second average associative binding constants are significantly different in value by at least one least significant difference (LSD) at an 80% confidence level; wherein said first and said second average associative binding constants are significantly lower in value than the magnitude of the average associative binding constant of the target imprintable entity (TIE) used to imprint either one of said molecularly imprinted polymers; wherein at least two of said sets of binding sites are formed during a polymerization process using at least one second polymerization aid than is different than a first polymerization aid employed in the formation of said first set of binding sites; wherein said second polymerization aid is selected from a different target imprintable entity, a different porogen, a different solvent, a different cosolvent, a different pore modifying agent, or combinations thereof; (c) optionally, associating at least one of said molecularly imprinted polymers with a time-delay factor that operates to delay the exposure of said at least one molecularly imprinted polymer it is associated with for a desired period of time after contact with a fluid media; wherein said molecularly imprinted polymer system operates to provide the programmed catch and/or release of said material into said fluid media.

One further aspect of the present disclosure is a molecularly imprinted polymer system for use in the controlled release of a medicinal agent in the presence of a contra-indicated substance, comprising: (a) a first molecularly imprinted polymer matrix templated with at least one molecular recognition pattern corresponding to said contra-indicated substance that operates to strongly catch or bind said substance upon contact; (b) a second molecularly imprinted polymer matrix with at least one or a plurality of suboptimum associative binding constants with respect to said medicinal agent; wherein said second molecularly imprinted polymer matrix is preloaded with said medicinal agent after formation and extraction of a suitable templating material; (c) optionally, a time-delay coating around said second molecularly imprinted polymer matrix bearing said preloaded medicinal agent; wherein said coating is effective in shielding said second molecularly imprinted polymer matrix for a desired time period; wherein said second molecularly imprinted polymer matrix with said at least one or a plurality of suboptimal associative binding constants operates to controllably release the preloaded medicinal agent at a controlled rate into a fluid media; and wherein said optional time-delay coating operates to enable said first molecularly imprinted matrix to adsorb said contra-indicated substance from said fluid media prior to the release of said medicinal agent.

### GENERAL EMBODIMENTS

In one general embodiment of the present disclosure, a molecular imprinted polymer (MIP) that is imprinted with a first target imprintable entity (TIE) using porogens, solvents, and polymerization conditions selected to produce binding sites exhibiting at least one modified average associative binding constant (i.e., kₘ<k_{TIE}) with respect to a second material (m) intended to be absorbed, exchanged or released from the MIP, can be designed, produced and used to control that second material's rate of release and desired release profile, the rate of adsorption and desired adsorption profile, and combinations thereof.

In a second general embodiment of the present disclosure, a molecular imprinted polymer (MIP) that is imprinted with a first target imprintable entity (TIE) using porogens, solvents, and polymerization conditions selected to produce binding sites exhibiting at least one modified average associative binding constant (i.e., kₘ<k_{TIE}) with respect to a second material (m) that is intended to be absorbed, exchanged or released from the MIP, can be designed, produced and used in selected combinations with time-delay release materials associated with the novel MIPs to control that material's rate of release and desired time-delayed release profile, the rate of adsorption and desired time-delayed adsorption profile, and combinations thereof.

In a third general embodiment of the present disclosure, a molecular imprinted polymer (MIP) that is imprinted with one or more target imprintable entities (TIE) using porogens, solvents and polymerization conditions selected to produce a plurality of binding sites exhibiting at least two or more modified average associative binding constants (i.e., kₘ₁≠kₘ₂, . . . kₘ₁₀<k_{TIE1}) with respect to a second material (m) intended to be absorbed, exchanged or released from the MIP, can be designed, produced and used to control that material's rate of release and desired release profile, the rate of adsorption and desired adsorption profile, and combinations thereof.

In a fourth general embodiment of the present disclosure, a molecular imprinted polymer (MIP) that is imprinted with one or more target imprintable entities (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE1}) with respect to a second material (m) intended to be absorbed, exchanged or released from the MIP, can be designed, produced and used to achieve predetermined release and adsorption profiles exhibiting zero-order, first-order, second-order, increasing ramp profiles, decreasing ramp profiles, exponential, geometric and polynomial profiles, and combinations thereof.

In a fifth general embodiment of the present disclosure, a molecular imprinted polymer (MIP) that is imprinted with a first target imprintable entity (TIE) using porogens, solvents, and polymerization conditions selected to produce binding sites exhibiting at least one modified average associative binding constant (i.e., kₘ<k_{TIE}) with respect to a second material (m) that is intended to be absorbed, exchanged or released from the MIP, can be designed, produced and used in selected combinations with time-delay release materials associated with the novel MIPs to achieve predetermined delayed release and delayed adsorption profiles exhibiting delayed zero-order, first-order, second-order, increasing ramp profiles, decreasing ramp profiles, exponential, geometric and polynomial profiles, and combinations thereof.

In a sixth general embodiment of the present disclosure, a molecular imprinted polymer (MIP) that is imprinted with one or more target imprintable entities (TIE) using porogens, solvents, and polymerization conditions selected to produce a plurality of binding sites exhibiting at least one modified average associative binding constants (i.e., kₘ<k_{TIE1}; kₙ<k_{TIE2}) each with respect to a second material (m) and a third material (n), which then operates to independently control both the second and the third material's rate of release and desired release profile, the rate of adsorption and desired adsorption profile, and combinations thereof, following independently determined profiles corresponding to zero-order, first-order, second-order, increasing ramp, decreasing ramp, increasing step, decreasing step, exponential, geometric, polynomial profiles, and combinations thereof, independently for both the second material and the third material.

In a seventh general embodiment of the present disclosure, a molecular imprinted polymer (MIP) that is imprinted with one or more target imprintable entities (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE1} and; kₙ₁≠kₙ₂, . . . kₙ₁₀<k_{TIE2}) with respect to a second material (m), a third material (n) and in further combination with selected time-delay release materials associated with the novel MIP, which then operates to independently control both the second and third material's rate of release and desired time-delayed release profile, the rate of adsorption and desired time-delayed adsorption profile, and combinations thereof, following independently determined desired profiles corresponding to delayed zero-order, delayed first-order, delayed second-order, delayed ramp, delayed step, delayed exponential, delayed geometric, delayed polynomial profiles, and combinations thereof, independently for both the second material and the third material.

In an eight general embodiment of the present disclosure, a molecular imprinted polymer (MIP) that is imprinted with one or more target imprintable entities (TIE) using porogens, solvents, and polymerization conditions selected to produce a plurality of binding sites exhibiting at least one modified average associative binding constants (i.e., kₘ<k_{TIE1} and kₙ<k_{TIE2}) each with respect to a second material (m) and a third material (n), which then operates to independently control both the second and the third material's rate of release and desired release profile, the rate of adsorption and desired adsorption profile, and combinations thereof, following independently determined profiles corresponding to zero-order, first-order, second-order, increasing ramp, decreasing ramp, increasing step, decreasing step, exponential, geometric, polynomial profiles, and combinations thereof, independently for both the second material and the third material, and with respect to the exchange of the second and third material between a media and a MIP matrix in contact with the media, where the media includes a gas, a liquid, a fluid, a neat liquid material, a solution, a composition, aqueous and non-aqueous solutions, a vapor, a liquid film, a wetted interface, a wetted surface, a biological system, and combinations thereof, as well as other media disclosed herein.

In one embodiment of the present disclosure, a molecular imprinted polymer that is imprinted with a first target imprintable entity (TIE) is selected that has at least one modified average associative binding constant with respect to a second material (i.e., kₘ<k_{TIE}) intended to be released, can be designed, produced and used to control insects by means of slowly releasing an insecticidal material to an air space, water supply, a surface or the like. For example and without limitaiton, a novel MIP made into the form of or incorporated into bed linens, protective nets or window screens could be produced using a selected TIE, which is then extracted from the MIP, which in turn is then saturated with an insecticide or insect repellant such as DEET (N,N-Diethyl-meta-toluamide).

With the proper selection of the TIE and polymerization conditions used to form and imprint the MIP, the present disclosure enables the selective design and production of a MIP having one or more of a plurality of sets of binding sites wherein the average associate binding constant is modified and suboptimal with respect to the material to be released, here DEET (i.e., kDEET<km) and which then operates to release the insecticide at a predetermined desired rate and release profile over a desired time period. The novel MIP matrix could then be recharged by washing in the presence of the insecticide or direct application of the insecticide to the MIP matrix in neat form or the form of a solution, with insecticide sufficiently applied so as to saturate or fill a substantial majority of available binding sites within the MIP matrix, which would then operate to controllably release the insecticide, be recharged, re-used, etc., repeatedly. In other embodiments, other insecticides and combinations thereof could similarly be employed using the methods of the present disclosure, and the desired rate of release and release profile of any particular material could be achieved by use of the novel approach to design a control release MIP by proper selection of the TIE and polymerization conditions employed to produce one or more of a plurality of material binding sites within the MIP exhibiting modified and suboptimal average associative binding constants with respect to the material to be regulated.

In a related embodiment to that immediately above, the novel MIPs could be in the form of a MIP matrix having a plurality of modified binding sites having two or more sets of average associative binding constants with respect to a selected medicant or material to be dosed, so that the MIP matrix would operate to release the medicant in a controlled fashion according to a desired time release profile whose characteristics are determined by the selection of the sets of average associative binding constants, each of which exhibit a k_{MIPm} that is less than and significantly different that the k_{Optimal} or k_{TIE} value with respect to the selected material; and wherein each k_{MIPm} is significantly different in value that every other average associative binding constant, would operate to release the insecticide at a selected rate and release profile over a desired time period. In a closely related embodiment to this, the novel MIPs could be in the form of a MIP system, in which two or more of the novel MIP matrices, each having a characteristic modified and suboptimal binding site or plurality thereof, are combined in order to operate together to achieve a desired release or desired catch profile with respect to a medicant or material to be released into a fluid media, and a material to be caught or removed from the fluid media, respectively.

In a further related embodiment to that immediately above, the novel MIPs could employ a plurality of modified binding sites having two or more sets of average associative binding constants with respect to an medicant or material to be dosed, and the MIPs either combined or separated being subsequently coated with a time-delay release coating or dissolvable barrier providing a time release delay function, so that the resulting delay release MIP matrix would operate to release the medicant in a controlled fashion according to a desired time release profile whose characteristics are determined by the selection of the sets of average associative binding constants and the time delay characteristics of the one or more time-delay release coatings employed. In these particular novel embodiments, an initial low or high level dosage rate of a medicant could be achieved, followed by a change in the release rate to a second low or high level dosage rate, or alternatively a change in the release rate according to a step-function or ramp-function change in rate over time, and combinations thereof, as desired.

In one further embodiment of the present disclosure, a molecular imprinted polymer with at least one modified average associative binding constant with respect to a pesticidal, antibiotic or antimicrobial material can be designed, produced and used to release the desired material in combination with a second MIP having catching characteristics and that have been imprinted with one or more molecular species common to the surface of a selected parasitic organism, such as for example but not limited to, surface proteins, surface enzymes, glycoproteins, sugars, and other biochemical entities present on the exterior surfaces of a selected organism's cell wall or protein sheath. In this example embodiment, the combined novel MIP matrix would operate to provide the controlled or time release of an antimicrobial or antibiotic agent, for example, while simultaneously operating to strongly adsorb and catch the selected individual parasitic organisms by means of strongly binding to molecular species on the surfaces of the parasites. In a specific example, a further embodiment to that described immediately above would be incorporating the novel MIP systems into a filtering system for rendering contaminated water potable, such as the LIFESTRAW, in which the novel MIP system could controllably and over time release an antimicrobial material such as, but not limited to, an organic chlorine-releasing material, a hypohalite, sodium dichloroisocyanurate, chloramine-T and the like, into the filtered water in a controlled release manner to prevent the over dosage or consumption of excess antimicrobial, while the novel MIP system simultaneously binds and catches rotavirus from the filtered water stream owing to at least one of the MIP matrices including a MIP imprinted to recognize one or more molecular species common the surface of infective rotaviruses being targeted for removal and treatment.

In a related embodiment, the MIP system could be fashioned into or added to a water filtration means, the novel MIPs selected so as to enable the controlled release into the treated water of a disinfectant or water sterilizing active, such as, but not limited to, an organic chlorine-releasing material, a hypohalite, sodium dichloroisocyanurate, chloramine-T and the like, operating to make the treatment water potable, or safe for consumption.

In yet a further embodiment of the present disclosure, a molecular imprinted polymer with at least one modified average associative binding constant with respect to an malarial antimicrobial or anti-malarial agent can be designed, produced and used to release that antimicrobial or agent in combination with a second MIP having catching characteristics that has been imprinted with one or more molecular species common to the surface of the malarial parasitic organism, such as for example but not limited to, surface proteins, surface enzymes, glycoproteins, sugars, and other biochemical entities present on the exterior surfaces of malarial protozoan cell walls. In this example embodiment, the combined novel MIP matrix would operate to provide the controlled or time release of a malarial antimicrobial and/or an anti-malarial agent, while simultaneously operating to strongly adsorb and catch individual protozoan and parasitic species associated with malarial infections by means of strongly binding to molecular species on the surfaces of the parasites. Thus, this example embodiment, ingested by a mammal or human, and in the form of a MIP particle, fiber, film or other suitable physical form compatible with introduction into the bloodstream or by ingestion, would operate to adsorb and remove the actual malarial parasites from the fluid media as well as operating to controllably release an antimicrobial and/or an anti-malarial agent in that same fluid media providing a dual protective benefit.

In other related embodiments, the novel MIPs could target other disease organisms and disease organism released toxins, while providing controlled release of antimicrobials and anti-parasitic agents targeting other organisms, microbes, viruses, prions, eukaryotes, bacteria, archaea, and other infective materials and the like, in polymer matrices comprising the novel MIPs in any suitable form enabling ingestion, injection, inhalation, insertion, incorporation and/or application to an animal or human exposed to one or more disease organisms or toxins thereof. In an novel example of this immediately preceding embodiment, the novel MIPs could be formed into a fiber or incorporated into a fiber for use as a suture for sewing and closing surgical sites and wounds. One or a plurality of the novel MIP matrices having one or more sets of modified and suboptimal average associative binding constants with respect to a selected antimicrobial compound could be employed to affect the extended and controlled release of that material while the sutures are in place and exposed to bodily fluids, in order to maintain a steady or constant level of antimicrobial compound released into the fluid media in contact with sutures incorporating the novel MIPs. In a further embodiment, the novel MIPs could be selected and combined in a MIP system having two distinct types of novel MIP matrices present, one for example providing the controlled release of an antimicrobial and a second providing the controlled release of a coagulating agent, for example, so that when used in the form of a suture, the included novel MIPs would operate to release two different medicants, each at its own unique selected rate or unique release profile over a selected time period. In a related embodiment, the present novel MIP matrix is tailored to have one or more sets of modified binding sites enabling the controlled release of an anti-inhibitor coagulant complex material, such as for example, but not limited to Vitamin K, prothrombin, thrombin activating factors VII, VII, IX, X and XI, their commercially available versions including Autoplex™ T, Feiba™ NF, Feiba™ VH Immuno™ and combinations thereof.

In a further related embodiment, the novel MIP system described immediately above are incorporated into fibers for use in bandages, wraps, swabs, surgical drapes, pads, wipes and other textile or fiber-based products used in the treatment of wounds, surgical sites, abrasions, cuts, scrapes and other injured sites of a mammal, the novel MIPs operating to deliver one or more time-delayed medicants for controlling infective agents while optionally, simultaneously operating to adsorb and bind one or more infective agents themselves or one or more toxic byproducts or metabolites released by the selected infective agent.

In another related embodiment to control vascular restenosis, the novel MIPs could be fashioned into, coated onto or otherwise incorporated into a medical insert such as a coronary stent, employing a control release MIP that has been fashioned to deliver extended and controlled time release of selected medicines such as anticoagulant drugs and scar tissue reducing agents that prevent restenosis, and do so in the immediate locality of the emplaced insert for maximum effectiveness. In this embodiment, the novel MIPs could provide for reliable, extended and controlled time release of FDA-approved anti-restenosis factors including Paclitaxel, Taxol, Rapamycin (macrolide antibiotic), as well as other antiplatelet agents, anticoagulants, anti-inflammatory agents, hypolipidemic agents, ACE inhibitors, calcium antagonists and antioxidants, and combinations thereof.

In yet another further related embodiment, the novel MIP system described immediately above could be fashioned into the form of bristles for use in a toothbrush, or in the form of fibers or string in floss, or incorporated into material forming a dental pick or a flossing device for cleaning between teeth and other related dental instruments, the novel MIPs tailored to deliver a time-delayed dosage of an anti-bacterial, or anti-halitosis, anti-carries or anti-plaque effective agent during use by means of employing one or more MIP matrices to release an effective agent(s).

In yet a further related embodiment, the MIP system described immediately above could further be used in combination with another MIP present and imprinted so as to bind and catch one or more selected bacterial species known to be associated with the disease condition being treated, so that the ensemble or MIP system operates to reduce the bacterial population in the mouth and around the teeth during a cleaning operation, and simultaneously provides a measured release of an effective agent to the mouth and tissues during use, saliva acting as a fluid media to transport materials from and into the MIP system, for example.

In another embodiment of the present disclosure, a molecular imprinted polymer with at least one modified average associative binding constant with respect to a pharmaceutical drug can be designed, produced and used to deliver that drug selectively by means of an novel “payload” MIP in combination with a “recognition” MIP that has been imprinted with and operates to target a specific cell or particular cellular surface feature associated with a disease condition of that cell, optionally including a delay element associated with the novel MIP to delay the release of the drug for a predetermined time period after introduction of the combination of MIPs into a mammal, for example, to affect treatment of a cellular based disease such as cancer, tuberculosis, and the like, the time-delay enabling the combination of MIPs (MIP complex) to circulate through the body and for the recognition MIP to become anchored at the desired treatment site, before the drug is substantially released from the payload MIP.

In yet another embodiment of the present disclosure, a molecular imprinted polymer with at least one modified average associative binding constant with respect to a pharmaceutical material to help control weight can be designed, produced and used to deliver a time-delay dosage of a material capable of blocking fat transport to adipose or vascular cells. In this example, an novel MIP matrix is templated to have one or more binding sites with modified average associative binding constants with respect to an expression vector material (typically a short amino-acid sequence) that binds to the FABP4 gene that modulates adipose fat storage in mammalian cells via the expressed enzyme prohibitin. The novel MIP matrix is saturated with the expression vector material and is then paired with a second MIP that has been templated with a nine amino acid adipocyte targeting sequence (ATS) that is specific to prohibitin and thus will operate to bind to the enzyme in situ upon contact. Both the novel MIP matrix and the ATS-templated recognition MIP, in the form of a MIP complex, are then reduced to nanoscale sizes, approximately to the 100-200 nanometer size range suitable for ingestion or injection into the digestive track or blood stream of a mammal undergoing treatment and thus capable of being taken into and circulated by means of the blood and/or lymphatic system. Eventually, circulating MIP complexes within the mammalian body contact and strongly bind to a prohibitin enzyme molecule located in the vicinity of a adipose fat cell, interfering with its function by means of binding to the enzyme at a selected recognition site, preferably near an active site of the enzyme required for functionality, and the novel MIP matrix component of the MIP complex then releasing the FABP4 gene interfering expression vector material in the vicinity of the adipose cell, which absorbs the expression vector material and which in turn shuts down the gene expressing the prohibitin enzyme production, resulting in the concerted interference with, and reduced production of the enzyme, resulting in reducing the amount of free fats transported to adipose or vascular cells, and thus reducing fat storage in adipose or vascular cells within the effective vicinity of the treatment area where the recognition MIP component of the MIP complex has located. In further embodiments of this novel example, the MIP matrix could be a tethered collection of the novel MIP and recognition MIP materials in the form of nano-sized particles with a covalent chemical bond attaching them, and optionally, wherein the covalent chemical bond is one that is susceptible to eventual breakage, such as for example, but not limited to an ester bond which will eventually hydrolyze and enable the MIP complex to break apart after it has operated to bind to and release its payload to targeted tissue, and the MIP components then released back into the bloodstream and eventually filtered therefrom and excreted from the treated mammal.

In yet another related embodiment to that described immediately above, the MIP complex could further include a delay release coating on the novel MIP matrix component, selected from a suitable material that would slowly dissolve under biological conditions over a desired time period and then operating as described herein to temporarily shield the novel MIP matrix and prevent the start of the release of its payload until the MIP complex has had sufficient time within the circulatory system of the mammal being treated to locate at the desired position by means of the associated recognition MIP matrix, and then operate to release its payload when the delay release coating decays or dissolves sufficiently to expose the novel MIP matrix to the local cellular environment.

In an embodiment of the present disclosure, a molecular imprinted polymer with at least one modified average associative binding constant with respect to an anti-cancer drug or cancer treatment agent can be used in combination with a recognition MIP to target cancer cells in a human or animal and then release its payload. In this embodiment, all the MIP components are utilized that are in the nano size range, such as a nanoparticle having a diameter of around 100 to 200 nanometers. An novel MIP matrix preloaded with a drug that kills cancer cells is programmed to have a desirable controlled release profile sufficient to deliver the drug over a selected time period, and the drug-laden MIP matrix is then coated with a time-delay coating with sufficient properties to delay the exposure of the novel MIP matrix for a desired time. Then, the coated MIP matrix is combined, for example by either physically attaching or chemically linking, to a recognition MIP that has been templated with a recognition material that is representative of some unique protein or cellular material associated with a cancer cell, so that in the form of a nanoparticle complexed to the novel drug releasing MIP, the MIP complex will eventually bind to a cancer cell after being introduced to the body of a human or animal, and as the delay-release coating around the payload MIP dissolves, release the anti-cancer drug or cancer treatment agent locally at that site, operating to weaken or kill the cancer cell preferentially owing to delivery of the desired drug or agent near the targeted cell. Then, over time, the novel MIP complex would dissociate or be swept back into the blood stream upon disintegration of the targeted cell, and eventually be excreted from the treated human or animal.

In a further related embodiment to that described immediately above, an novel MIP matrix component could be designed and produced to release an RNA interference vector (RISC) to stop production of cells associated with cancer by suppressing expression of a gene associated with that vector, for example, but not limited to an novel MIP matrix programmed to controllable release RNA interference vectors targeting gene sequences such as HMGA1 for breast cancer cells, CELF1 for lung cancer cells, EGFR for gastric cancer cells, eIF3c for colon cancer cells, ICB-1 for ovarian and breast cancer cells, and the like, as well as combinations thereof.

In yet a further related embodiment to that described immediately above, an novel MIP complex could further include a “catching” MIP, optionally including a time-delay coating, that has been templated with the actual anti-cancer drug, cancer treatment agent or interference vector, so as to operate, when subsequently exposed to the cellular environment upon dissolution or breaching of the time-delay coating, to then strongly and optimally adsorb all free and accessible previously released drug, agent or vectors to prevent their spreading to tissues outside of the vicinity of the targeted cell.

In a further embodiment, a second novel MIP matrix could be used in combination with the MIP complex described in the embodiment immediately above, having a least one modified associative binding constant with respect to the payload material selected, so that it would operate as a “scavenging” MIP matrix to controllably adsorb excess drug, agent or vector materials released from the first novel payload delivery MIP matrix, but at a slower adsorbing rate than the release rate exhibited by that first MIP matrix, so that the concentration of the treatment material is able to build up to an effective dosage level in the vicinity of the targeted cell, and then be scavenged by the second novel MIP matrix which operates to reduce the treatment material concentration at a later time and thus prevent migration of excess amounts of treatment material from the vicinity of the cell to which the MIP complex has become attached or become associated with.

In yet a further embodiment, the second novel MIP scavenging matrix described immediately above could also be coated with a time-delay coating designed to dissolve or become breached after a time period greater than the time-delay coating, if used, or a time period sufficient to enable the substantial quantitative release of the treatment agent by a first novel MIP system, so that the treatment agent is enabled to function for a selected period of time without interference, and any excess material remaining is then scavenged by the second novel MIP matrix after the selected time period associated with its unique time-delay coating has passed.

In another embodiment relating to contraception and sexually transmitted disease control, a condom, diaphragm, cervical plug, cervical shield, sponge or other similar device to be inserted into a vaginal cavity is constructed from or combined with an novel MIP system that operates to simultaneously release a spermicidal agent, contraceptive or antimicrobial active while also operating to adsorb a selected pathogen into and from the vaginal environment. By means of the novel MIP systems described herein, controlled time-delay of a spermicidal agent, contraceptive or antimicrobial active can be achieved to deliver a first specific dosage or first release rate and maintain that initial level or rate, and optionally in combination with a time-delay functionality, can further be designed to achieve a second level or second release rate and maintain that second level or rate for a second period of time, for example.

The example novel MIP system may be combined with a recognition MIP, being a MIP that has been imprinted with one or more characteristic molecular entities associated with a particular pathogen's exterior cellular surface or membrane, which operates to bind the pathogens to accessible sites within the recognition MIP, reducing media concentration levels of the pathogen, and thus reducing the spread of germs and lowering the chances of infection and disease transmission. In further embodiments related to this example, the novel MIP systems could include recognition MIPs targeting for example the homologous type-common surface glycoprotein-D residues of Herpes Simplex Virus Types 1 and 2. In yet further embodiments related to this same example, the novel MIP systems could include recognition MIPs targeting other sexually transmitted disease organisms via a similar mechanism, including such pathogens, but not limited to AIDS, HPV, hepatitis, bacterial vaginosis, chlamydia, trichomoniasis, gonorrhea, syphilis, and combinations thereof.

In a related embodiment, the novel MIPs may be tailored to control the population of Candida albicans (yeast fungus) and Gardnerella vaginalis, and combinations of the two, both leading causes of vaginitis, by imprinting a MIP matrix with the organisms or selected cellular membrane materials characteristic to the two organisms, to produce binding sites having suboptimal associative binding constants so that the novel MIP matrix or system operates to controllable limit and reduce the level of the organisms present in fluid media in locations such as the vagina and cervix, but not completely bind and immobilize all of the organisms present. Such MIP matrices could be fashioned into webs for use in tampons and similar devices, or otherwise fashioned into diaphragms, sponges, shields, condoms, and the like for temporary insertion or prolonged emplacement within a vaginal cavity. Thus, the novel MIPs may be used to control the catching (adsorption) and release of live organisms in a manner similar to how the present disclosure operates to recognize and bind other chemical and other biological materials, by selection of a MIP exhibiting two or more average associative binding constants with respect to the organism or a recognition site present on the exterior cell or membrane surface of the target organism. In this manner, the novel MIPs operate to maintain a healthy level of organisms present, preventing toxic shock syndrome or excessive culling of the population, acting instead to maintain a reduced, sub-colonization level of organisms present. In further embodiments, the novel MIPs described immediately above may be combined with additional novel MIPs and MIP matrices that have been designed and selected to affect a desired controlled and time-delay dosage of a medicant to maintain vaginal health, examples including, but not limited to, anti-vaginosis drugs, pH buffers, antimicrobials, antifungal agents, yeast colony factor inhibitors, hormones, estrogen, testosterone, epithelial growth and repair factors, and the like, and combinations thereof.

In one embodiment, the novel MIPs may be formed into or combined with contact lenses or the like to produce therapeutic contact lens, patches, films, ocular inserts, intraocular inserts, intravitreal inserts, punctal implants, treatment ointments, lotions, drops and solutions, and combinations thereof, that operate to controllable deliver a therapeutic material to the eye or ocular cavity as programmed to achieve a desired release rate, release rate profile, and combinations thereof. In this example, FDA-approved ocular topical medicants, including but not limited to Bromfenac (NSAID), Bepotastine (Talion, an antihistamine), Besifloxacin (fluoroquinolone antibiotic), Ganciclovir (antiviral), Loteprednol etabonate (corticosteroid), Fluocinolone acetonide (corticosteroid), Timolol (beta-adrenergic receptor antagonist for glaucoma), Macugen and combinations thereof, could be controllable dosed as desired to treat a variety of eye diseases selected from, but not limited to allergies, dryness, irritation, redness, Age Related Macular Degeneration (AMD), allergic conjunctivitis, bacterial conjunctivitis (Pink Eye), corneal edema, Dry Eye Syndrome (DES), glaucoma, viral conjunctivitis and the like, and combinations thereof.

In a further embodiment, the novel MIPs may be formed into or combined with textile materials fashioned into a range of fabrics, clothing, linens, swabs, wraps, bandages, pillow cases, coverings and the like, the MIPs tailored to deliver a controlled release dosage of one or more materials effective in controlling the spread of germs. Suitable materials include for example, but are not limited to, primary antimicrobials, disinfectants, bacteriostats, antivirals, anti-colonization signally factors, and combinations thereof, to prevent the spread of nosocomial infections. In operation, such novel MIP materials would operate to release their payload material when the MIP is exposed to a liquid or biological contaminant or secretion, such as condensed breath, nasal secretions, blood, lymph, plasma, bodily secretions, semen, sweat, spit, snot, tears, urine, pus, vomit, and the like.

In another embodiment, the novel MIPs are tailored to deliver timed release of a plant hormone that will accelerate the growth of plants, promote fruiting and/or ripening. Fashioned into the form of beads or pellets, a “payload” MIP containing for example, but not limited to gibberellins, could be exploited as a soil amendment agent to release the material over time. In a preferred embodiment, the polymers used to produce the novel MIP would be biodegradable, so that at some time after the novel MIPs have served their purpose, the remaining MIP materials would eventually be broken down and degraded by soil bacteria present and leave no environmental trace or residue behind.

In an novel embodiment relating to personal care, the novel MIPs could be designed to affect the time release of a hair growth stimulant, such as for example, but not limited to ROGAINE. Tinted by a suitable dye to match a person's desired hair color, the novel MIP matrix could be fashioned into the form of small hair-like fibers or adherent nano-fibers that could be applied, sprayed or sprinkled onto thinning hair or balding regions of the skin. In the presence of moisture (sweat, humidity), the novel MIP would operate to release the therapeutic material to the scalp and hair follicles, while temporarily tinting the treatment area and giving the appearance of hair being present at the treated locations.

In another novel embodiment relating to air treatment, the novel MIPs are fashioned into an air filtration device, such as an air filter, breathing filter, HVAC filter insert, filtering element, filter mask, and the like, the MIP matrix being used in the form of, for example but not limited to, a fabric sheet, fabric web, fiber web, non-woven matrix, filter disk, foam element, and the like. In these embodiments, the MIP matrix is tailored for the slow release of an air treatment chemical, such as for example but not limited to a volatile biocide, fragrance, perfume, scent, or other volatile material such as an essential oil. Alternatively, the MIP matrix is tailored for the slow release of another material, such as for example but not limited to a or a non-volatile biocide, fungicide, bactericide or the like, the latter which operates to prevent growth of microbes on the filter itself. In either of these embodiments the novel MIPs matrix could further be combined with a ‘targeting’ MIP that has been imprinted with one or more pathogens or molecular recognition fragments thereof, which operates to bind to the targeted pathogens and immobilize them in place on the filter element.

In a further related embodiment, the novel MIPs are fashioned into an air treatment device suitable for incorporation as a filtering element, flavoring element or insert associated with a cigarette or cigar style device which treats air inhaled by the user, the novel MIPs selected to deliver a time-delayed or controlled amount of a volatile material into the inhaled air stream, examples of such materials including, but not limited to nicotine, nicotine analogues, THC (tetrahydrocannabinol), THC analogues, cannabinol and cannabinoid analogues, flavoring agents, cough suppressant materials, analgesics, and the like, and combinations thereof.

In another related embodiment, the novel MIPs are fashioned into a dosage form for ingestion, such as for example, a pill or capsule of particulated MIP matrices, or a polymeric matrix suitable for transdermal delivery of a selected natural medicinal active, such as for example, but not limited to cannabinoid (CBD), cannabinol (CBC), tetrahydrocannabinol (THC), related compounds, isomers, hemp extracts, and the like, and combinations thereof, the novel MIP matrices operating to affect the controlled, time-delay delivery of the medicinal actives to a patient via the intestinal track, or through the skin, respectively. A particular advantage of using the novel, programmed time-delay MIP matrices described herein is that the drug or material to be released cannot easily be deliberately and prematurely released or separated from the MIP matrix, preventing the extraction, concentration and potential abuse of the selected drug or material, because crushing, mechanical degradation, separation or other physically destructive actions directed against the novel MIPs or MIP matrices does not alter the time-delay properties of the plurality of programmed, time-delay binding sites within the novel MIPs.

In a series of novel embodiments for the treatment of water, the novel MIPs are selected to provide the controlled time-delay of a material into a body or stream of water, such materials including for example but not limited to, nutrients, micronutrients, vitamins, flavors, enzymes, scents, taste modifiers, water softening materials, pH adjustment agents, buffering agents, and the like, and combinations thereof. In related embodiments, the above novel MIP systems could further be combined with a “catching” MIP selected to adsorb microbes, pathogens, toxins, undesired chemical elements, compounds, molecules and materials simultaneously from the filtered water source as the novel MIPs release their treatment agent or material into the water source. In another related embodiment, the above novel MIP systems could further be combined with a “catching” MIP selected to adsorb select toxic metals, such as aluminum, arsenic, chromium, copper, lead, mercury, and the like, having been either imprinted with the select metals or compounds thereof, or imprinted with metal binding compounds, such as, but not limited to chelants, sequestrants, chelators, polyanions, crown ethers, cationic sorbents, and the like, and combinations thereof, which may optionally be left intemplated within the formed MIP matrix, wherein the metal binding compounds operate to bind and remove select metal cations from the surrounding aqueous media.

In an example of a household product application, the novel MIPs are fashioned into or combined with a toilet treatment device that is placed in the tank, bowl or in contact with water within a toilet bowl, cistern, bidet or the like, the novel MIP component operating to deliver a controlled or timed-release of a selected material, such as for example but not limited to an antimicrobial agent, biocide, fragrance, scent, perfume, disinfectant material, oxidant, bleach, bleach activator, sequestrant, chelant, biofilm suppressing agent, cleaning aid, surfactant, buffer, pH adjusting material, visual indicator, dye and the like, and combinations thereof. In related embodiments, the novel MIP component could be further combined with a “catching” MIP selected to adsorb microbes, odors, malodors, pathogens, toxins, undesired chemical elements, compounds, molecules and materials simultaneously from the water source as the novel MIPs release their treatment agent or material into the water source.

In an example of a food preservation system, the novel MIPs are fashioned into the form of a coating, film, or insert in a food package, container or storage unit, the novel MIP component operating to deliver a controlled or timed release of a selected material, such as for example but not limited to an antimicrobial agent, biocide, anti-spoilage agent, buffer, pH adjusting material, preservative, anti-oxidant, free-radical scavenger, anti-corrosion agent, corrosion inhibitor, taste enhancer, and the like and combinations thereof into the package air space or into the foodstuff therein. In related embodiments, the novel MIP component could be further combined with a “catching” MIP selected to adsorb microbes, odors, malodors, pathogens such as botulism and the like, toxins such as botulinum, undesired chemical elements, compounds, molecules and materials simultaneously from the foodstuff or package as the novel MIPs release their treatment agent or material into the foodstuff or package.

### Objects of the Disclosure

One object of the disclosure is to design, produce and use a programmed molecular imprinted polymer (MIP) that is imprinted with a first target imprintable entity (TIE) using porogens, solvents, and polymerization conditions selected to produce binding sites exhibiting at least one modified average associative binding constant (i.e., kₘ₁<k_{TIE}) with respect to a second material (m), which then operates to control the second material's rate of release and desired release profile, the rate of adsorption and desired adsorption profile, and combinations thereof.

A second object of the disclosure is to design, produce and use a programmed molecular imprinted polymer (MIP) that is imprinted with a first target imprintable entity (TIE) using porogens, solvents, and polymerization conditions selected to produce binding sites exhibiting at least one modified average associative binding constant (i.e., kₘ₁<k_{TIE}) with respect to a second material (m), which then operates to control the second material's rate of release and desired release profile, the rate of adsorption and desired adsorption profile, and combinations thereof, with respect to the exchange of the second material between a media and a MIP matrix in contact with the media, where the media includes a gas, a liquid, a fluid, a neat liquid material, a solution, a composition, aqueous and non-aqueous solutions, a vapor, a liquid film, a wetted interface, a wetted surface, a biological system, and combinations thereof.

Another object of the disclosure is to design, produce and use a programmed molecular imprinted polymer (MIP) that is imprinted with a first target imprintable entity (TIE) using porogens, solvents, and polymerization conditions selected to produce binding sites exhibiting at least one modified average associative binding constant (i.e., kₘ₁<k_{TIE}), with respect to a second material (m) in further combination with selected time-delay release materials associated with the novel MIP, which then operates to control the second material's rate of release and desired time-delayed release profile, the rate of adsorption and desired time-delayed adsorption profile, and combinations thereof. In one aspect of the disclosure, the modified average associative binding constant exhibited by the second material is significantly lower in value than the average associative binding constant exhibited by the TIE material with respect to the novel MIP.

A further object of the disclosure is to design, produce and use a programmed molecular imprinted polymer (MIP) that is imprinted with a first target imprintable entity (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE}), with respect to a second material (m) which then operates to control the second material's rate of release and desired release profile, the rate of adsorption and desired adsorption profile, and combinations thereof. In one aspect of the disclosure, the plurality of modified average associative binding constants exhibited by the second material are each significantly different in value from each other, and are significantly lower in value than the average associative binding constant exhibited by the TIE material with respect to the novel MIP.

Another object of the disclosure is to design, produce and use a programmed molecular imprinted polymer (MIP) that is imprinted with a first target imprintable entity (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE}), with respect to a second material (m) in further combination with selected time-delay release materials associated with the novel MIP, which then operates to control the second material's rate of release and desired time-delayed release profile, the rate of adsorption and desired time-delayed adsorption profile, and combinations thereof.

Yet another object of the disclosure is to design, produce and use a programmed molecular imprinted polymer (MIP) that is imprinted with a first target imprintable entity (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE}) with respect to a second material (m), which then operates to control the second material's rate of adsorption and release following a desired profile corresponding to zero-order, first-order, second-order, exponential, geometric, increasing ramp profiles, decreasing ramp profiles, polynomial profiles, and combinations thereof.

One further object of the disclosure is to design, produce and use a programmed molecular imprinted polymer (MIP) that is imprinted with a first target imprintable entity (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE}) with respect to a second material (m), in further combination with selected time-delay release materials associated with the novel MIP, which then operates to control the second material's rate of release and desired time-delayed release profile, the rate of adsorption and desired time-delayed adsorption profile, and combinations thereof, following a desired profile corresponding to delayed zero-order, delayed first-order, delayed second-order, delayed ramp, delayed step, delayed exponential, delayed geometric, delayed polynomial profiles, and combinations thereof.

An additional object of the disclosure is to design, produce and use a programmed molecular imprinted polymer (MIP) that is imprinted with one or more target imprintable entities (TIE) using porogens, solvents, and polymerization conditions selected to produce a plurality of binding sites exhibiting at least one modified average associative binding constant (i.e., kₘ₁<k_{TIE1} and; kₙ₁<k_{TIE2}) each with respect to a second material (m) and a third material (n), which then operates to independently control both the second and the third material's rate of release and desired release profile, the rate of adsorption and desired adsorption profile, and combinations thereof, following independently determined profiles corresponding to zero-order, first-order, second-order, increasing ramp, decreasing ramp, increasing step, decreasing step, exponential, geometric, polynomial profiles, and combinations thereof, independently for both the second material and the third material. In one aspect of the disclosure, the plurality of binding sites for the second material exhibit modified average associative binding constants that are each significantly different in value from each other, and that are significantly lower in value than the average associative binding constant exhibited by the TIE material used to produce the binding sites for that second material with respect to the novel MIP; and the plurality of binding sites for the third material exhibit modified average associative binding constants that are each significantly different in value from each other, and that are significantly lower in value than the average associative binding constant exhibited by the TIE material used to produce the binding sites for that third material with respect to the novel MIP; and the no two sets of binding sites for either the second material and the third material exhibit the same average associative binding constant for the same material.

Another object of the disclosure is to design, produce and use a programmed molecular imprinted polymer (MIP) that is imprinted with one or more target imprintable entities (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE1} and kₙ₁≠kₙ₂, . . . kₙ₁₀<k_{TIE2}) each with respect to a second material (m) and a third material (n), which then operates to independently control both the second and the third material's rate of release and desired release profile, the rate of adsorption and desired adsorption profile, and combinations thereof, following independently determined profiles corresponding to zero-order, first-order, second-order, increasing ramp, decreasing ramp, increasing step, decreasing step, exponential, geometric, polynomial profiles, and combinations thereof, independently for both the second material and the third material.

Yet another object of the disclosure is to design, produce and use a programmed molecular imprinted polymer (MIP) that is imprinted with one or more target imprintable entities (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE1} and kₙ₁≠kₙ₂, . . . kₙ₁₀<k_{TIE2}) with respect to a second material (m), a third material (n) and in further combination with selected time-delay release materials associated with the novel MIP, which then operates to independently control both the second and third material's rate of release and desired time-delayed release profile, the rate of adsorption and desired time-delayed adsorption profile, and combinations thereof, following independently determined desired profiles corresponding to delayed zero-order, delayed first-order, delayed second-order, delayed ramp, delayed step, delayed exponential, delayed geometric, delayed polynomial profiles, and combinations thereof, independently for both the second material and the third material.

A further object of the disclosure is to design, produce and use a programmed molecular imprinted polymer (MIP) that is imprinted with one or more target imprintable entities (TIE) using porogens, solvents, and polymerization conditions selected to produce a plurality of binding sites exhibiting at least two or more modified average associative binding constants (i.e., kₘ₁≠kₘ₂, . . . kₘ₁₀<k_{TIE1} and kₙ₁≠kₙ₂, . . . kₙ₁₀<k_{TIE2}) each with respect to a second material (m) and a third material (n), which then operates to independently control both the second and the third material's rate of release and desired release profile, the rate of adsorption and desired adsorption profile, and combinations thereof, following independently determined profiles corresponding to zero-order, first-order, second-order, increasing ramp, decreasing ramp, increasing step, decreasing step, exponential, geometric, polynomial profiles, and combinations thereof, independently for both the second material and the third material with respect to the exchange of the second material and a third material between a media and a MIP matrix in contact with the media, where the media includes a gas, a liquid, a fluid, a neat liquid material, a solution, a composition, aqueous and non-aqueous solutions, a vapor, a liquid film, a wetted interface, a wetted surface, a biological system, and combinations thereof, and wherein said second material and third material are initially present in the MIP matrix, the media and combinations thereof.

Yet another object of the disclosure is the use of the novel MIP and MIP matrices as disclosed herein as components in combination with other MIPs providing molecular site recognition capability to achieve a MIP system which operates to cause the MIP system, when in a compatible form, to self locate to a desired and targeted site within a selected environment, enabling the novel MIP components to operate as disclosed herein to catch and/or release one or a plurality of independent materials at that site, the programmed molecular imprinted polymer (MIP) being imprinted with one or more target imprintable entities (TIE) under porogen, solvent and polymerization conditions selected to produce a plurality of binding sites exhibiting at least two or more modified average associative binding constants (i.e., kₘ₁≠kₘ₂, . . . kₘ₁₀<k_{TIE1} and kₙ₁≠kₙ₂, . . . kₙ₁₀<k_{TIE2}); and kₒ₁≠kₒ₂, . . . kₒ₁₀<k_{TIE3}, etc.), each with respect to a plurality of materials (m, n, o . . . ), and operating to independently control the various materials rates of release and desired release profiles, the rates of adsorption and desired adsorption profiles, and combinations thereof, following independently determined profiles corresponding to zero-order, first-order, second-order, increasing ramp, decreasing ramp, increasing step, decreasing step, exponential, geometric, polynomial profiles, and combinations thereof, independently for the various materials with respect to the exchange of those materials between a media and a MIP or MIP matrix in contact with the media, where the media includes a gas, a liquid, a fluid, a neat liquid material, a solution, a composition, aqueous and non-aqueous solutions, a vapor, a liquid film, a wetted interface, a wetted surface, a biological system, and combinations thereof, and wherein the various materials are initially present in the MIP or MIP matrix, the media and combinations thereof.

### BRIEF DESCRIPTION OF THE DRAWINGS

FIG. 1 shows a graph of a model release system.

FIG. 2A shows a graphical illustration of a novel MIP system.

FIG. 2B shows a graph corresponding to the illustration of FIG. 2A.

FIG. 3A shows a graphical illustration of a MIP system having an approximate equal number of two significantly different material binding sites.

FIG. 3B shows a graph corresponding to the illustration of FIG. 3A.

FIG. 3C shows a graph corresponding to the illustration of FIG. 3A.

FIG. 4A shows a graphical illustration of a MIP system having an approximate equal number of two different sets of material binding sites.

FIG. 4B shows a graph corresponding to the illustration of FIG. 4A.

FIG. 5A shows a graphical illustration of a MIP system having an approximate equal number of two different sets of material binding sites.

FIG. 5B shows a graph corresponding to the illustration of FIG. 5A.

FIG. 5C shows a diagram corresponding to a cross-sectional view of an oral dosage form.

FIG. 5D shows a diagram corresponding to a cross-sectional view of an another oral dosage from employing a first MIP matrix component and a second coated MIP matrix component.

FIG. 6A shows a graphical illustration of a MIP system having a dissimilar number of two different sets of material binding sites.

FIG. 6B shows a graph corresponding to the illustration of FIG. 6A.

FIG. 6C shows a graph corresponding to the illustration of FIG. 6A.

FIG. 7A shows a graphical illustration of a MIP system having two different sets of material binding sites.

FIG. 7B shows a graph corresponding to the illustration of FIG. 7A.

FIG. 8 shows a graph of a selected “step up” release profile.

FIG. 9 shows a graph of a selected initial high dosage steady-state release, followed by a step down to a subsequent delayed low dosage steady-state release profile.

FIG. 10 shows a graph of a selected initial steady state dosage release followed by a drop to a delayed low-to-high ramp increasing release dosage profile.

FIG. 11 shows one embodiment of an novel schematic process in diagrammatic form detailing the process for determining optimized parameter values for an novel MIP system.

FIG. 12A shows a result of modeling a novel MIP system in order to achieve a desired controlled, time-delay dosage profile.

FIG. 12B shows a result of modeling a novel MIP system in order to achieve a desired controlled, time-delay dosage profile.

FIG. 12C shows a result of modeling a novel MIP system in order to achieve a desired controlled, time-delay dosage profile.

FIG. 12D shows a result of modeling a novel MIP system in order to achieve a desired controlled, time-delay dosage profile.

FIG. 12E shows a result of modeling a novel MIP system in order to achieve a desired controlled, time-delay dosage profile.

FIG. 12F shows the root-mean-square (RMS) error of the successive novel MIP systems shown in FIGS. 12A-E compared to the desired dosage profile.

### DETAILED DESCRIPTION OF THE DRAWINGS

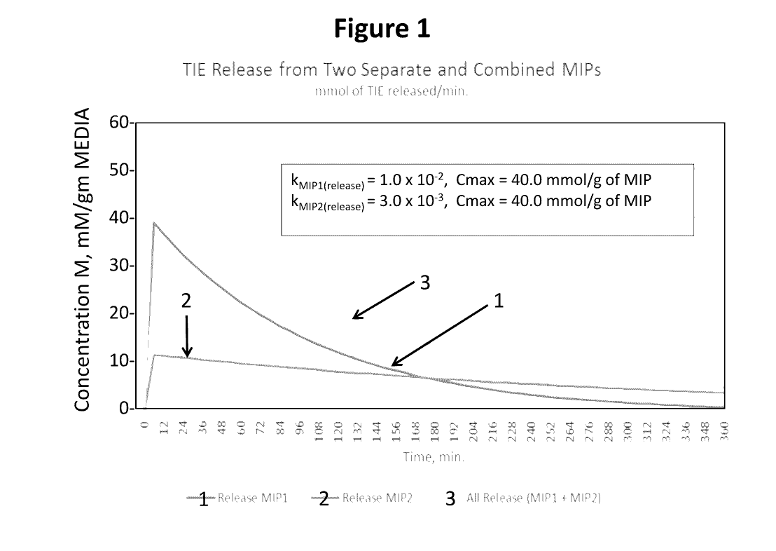

FIG. 1 shows a graph of a model release system being a combination of two MIP matrices each having a distinctive release rate with respect to a preloaded material, and the subsequent resulting overall (combined) release profile (concentration) of that material into a fluid media over time.

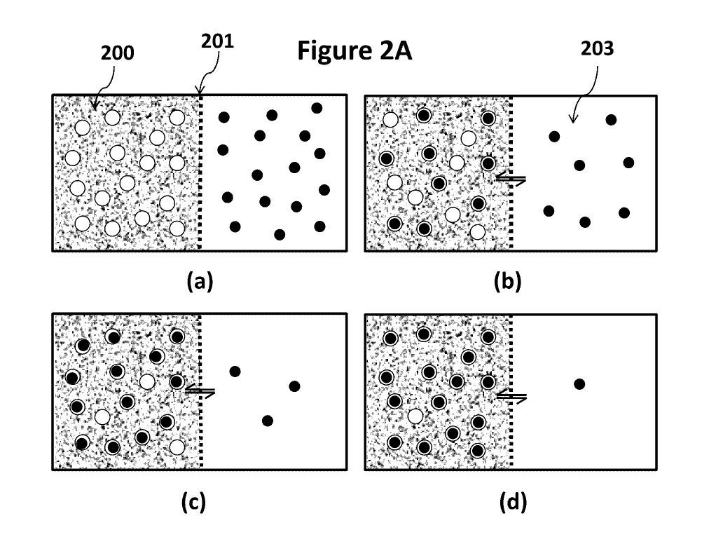

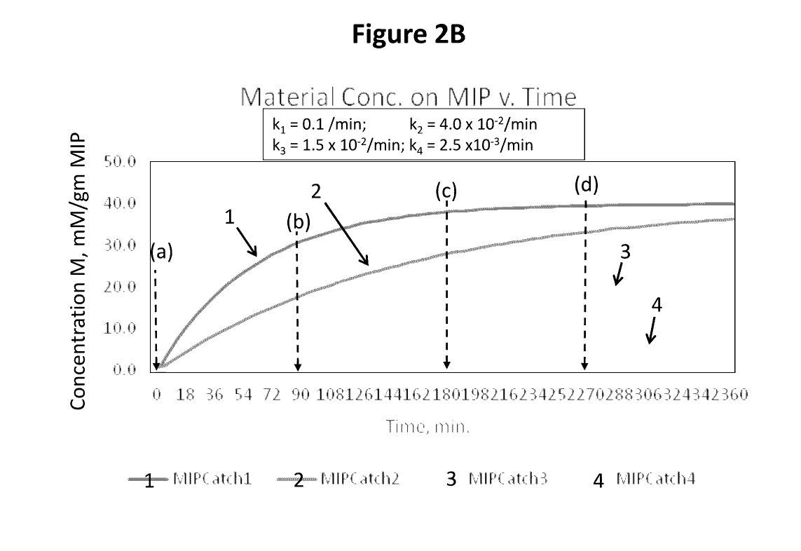

Note that in the following Figures, FIG. 2-7, graphical illustrations of the novel MIP systems are presented in which the MIP polymer matrix is illustrated on the left side of each rectangular frame (labeled (a)-(d)) as a shaded area (200 in FIG. 2A for example) with indicated binding sites either empty (white circles) or preloaded (circles with black dots) with a material (black dots). The vertical dotted line (201 in FIG. 2A for example) in the center of each frame illustrates that the entire surface of the MIP polymer matrix 200 shares an interface with the surrounding fluid media, which is indicated on the right side of each frame as an unshaded area (203 in FIG. 2A for example). A visual, representative number of material entities (black dots) are shown merely to illustrate the relative amount of the material present, either present in the fluid media or adsorbed into a corresponding binding site in the MIP polymer matrix and are intended to be non-limiting in anyway. Similarly, the number of material entities present are simply visual indicators provided to show the relevant extent of distribution of free and bound materials at arbitrary time frames starting at time zero (T₀) when the MIP polymer matrix is first exposed to the fluid media, and subsequent intermediate arbitrary time intervals of T₁, T₂ and finally T₃ representing an end point or the representative time at which the illustrated system has achieved an approximate state of equilibrium or steady-state behavior, the double-arrows intersecting the MIP surface boundary 201 with the surrounding fluid 203 media pictorially showing that the material can equilibrate between the MIP matrix 201 and the media 203.

FIG. 2A shows a graphical illustration of an novel MIP system (approximately 1.0 g weight) having an approximate equal number of material binding sites available (with a capacity of 40 mM/g, as shown by empty circles on the left side of the first frame (a) at an initial time zero (T₀), and at various later times T₁ in frame (b); T₂ in frame (c); and T₃ in frame (d); the MIP system being in contact with a fluid media in which a material is present at an initial starting concentration of 40 mM/L. The material is represented by the black dots.

FIG. 2B shows a graph corresponding to the illustration of FIG. 2A of the concentration (within the MIP) of a material adsorbed by 1 gm of the illustrated MIP system as a function of time from T=0 to T=360 min, where traces 1, 2, 3 and 4 show the adsorption profile of a MIP matrix having various average association binding constants with respect to the material.

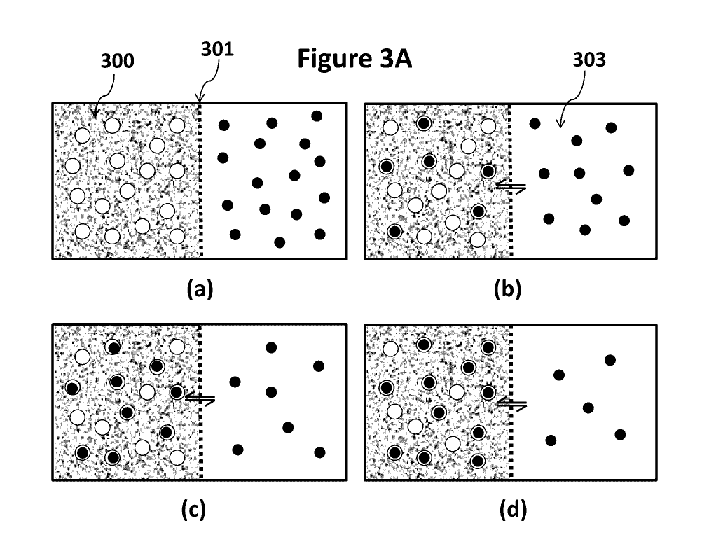

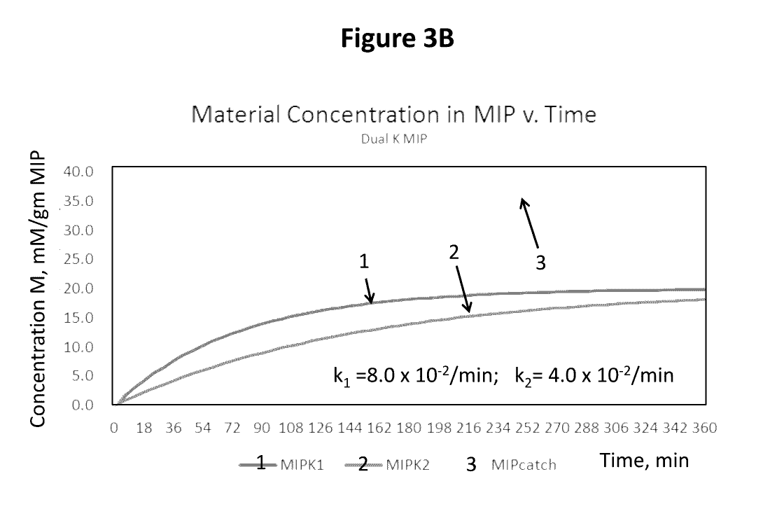

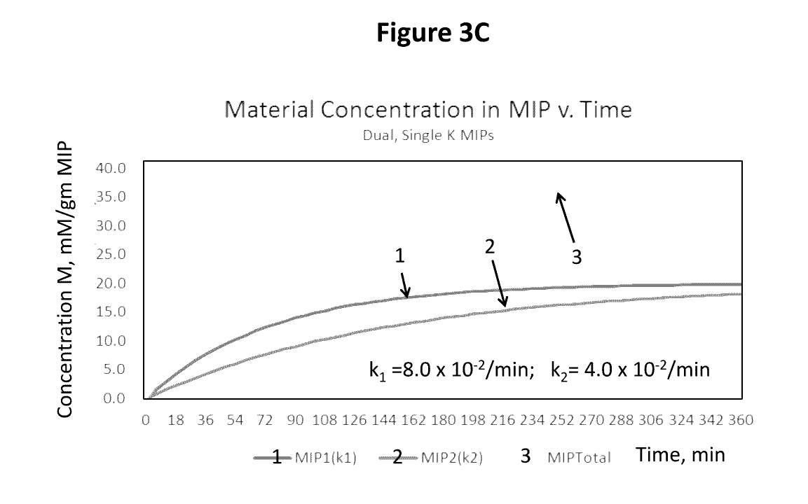

FIG. 3A shows a graphical illustration of a MIP system having an approximate equal number of two significantly different material binding sites (both shown by empty circles in frame (a) having k values of around 1.0×10⁻²/min and 5.0×10⁻¹/min, respectively, where the MIP system has a total material capacity of 20 mM/gm, shown initially at time zero (frame a); intermediate times (T1 and T2, frames b and c, respectively); and at equilibrium (T3, frame d), the MIP system being in contact with a fluid media in which the material is present at an initial starting concentration of about 50 mM/L. The material (molecular entity) is represented by the black dots.

FIG. 3B shows a graph corresponding to the illustration of FIG. 3A of the concentration of a material adsorbed by a MIP matrix (trace 3) as a function of time from T=0 to T=360 min, the MIP matrix having two unique kₘ values.

FIG. 3C shows a graph corresponding to the illustration of FIG. 3A of the concentration of a material adsorbed by a MIP system (trace 3) as a function of time from T=0 to T=360 min, the MIP system being composed of two MIP matrices, each individual MIP matrix having a unique kₘ value.

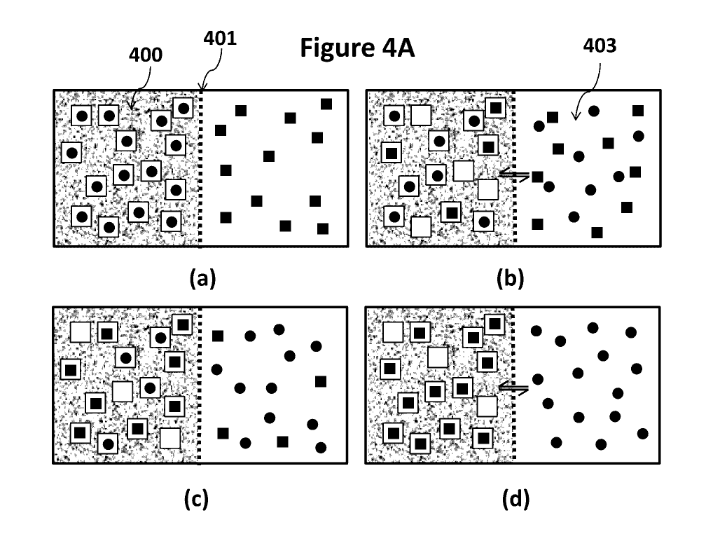

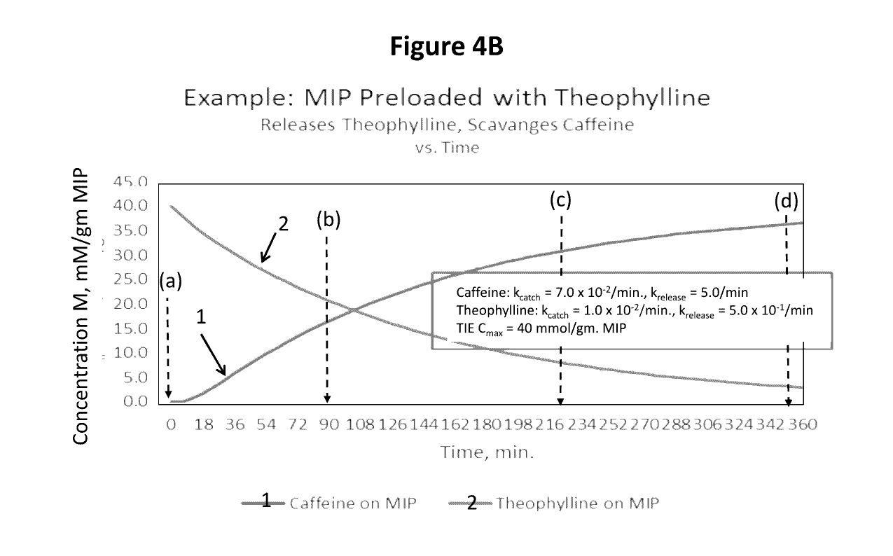

FIG. 4A shows a graphical illustration of a MIP system having an approximate equal number of two different sets of material binding sites (both shown by empty squares in frame a) selected to have significantly different k values for two different materials. One set of material binding site exhibits k values of 7.0×10⁻²/min and 5.0/min for a caffeine molecule; and a second set of material binding sites exhibits k values of 1.0×10⁻²/min and 5.0×10⁻²/min for a theophylline molecule, where the MIP system has a total material capacity of 40 mM/gm with respect to caffeine. Frame (a), shown initially at time zero (frame a), shows the MIP system preloaded with theophylline molecules occupying a substantial majority of MIP binding sites prior to contacting a fluid media that contains caffeine molecules present at an initial concentration corresponding to a total of about 40 mM of free caffeine. Frames (b)-(d) show illustrative times after the MIP system is contacted with the fluid media, which substantially reaches equilibrium at t=Tf corresponding to frame d. The MIP system remain in contact with the fluid media in which theophylline is present at an initial starting concentration within the MIP matrix of 50 mM/L. Here, theophylline molecules are represented as black circles (dots), while caffeine molecules are represented as black squares.

FIG. 4B shows a graph corresponding to the illustration of FIG. 4A (frames a-d) of the MIP concentration of two materials after the media has been contacted with 1 g of the example MIP system as a function of time from T=0 to T=360 min, where trace 1 shows the concentration of caffeine in the MIP matrix, trace 2 shows the respective concentration of theophylline in the MIP matrix, and the vertical dashed lines denoted as (a)-(d) approximately correspond to the time frames illustrated in FIG. 4A in frames (a)-(d), respectively.

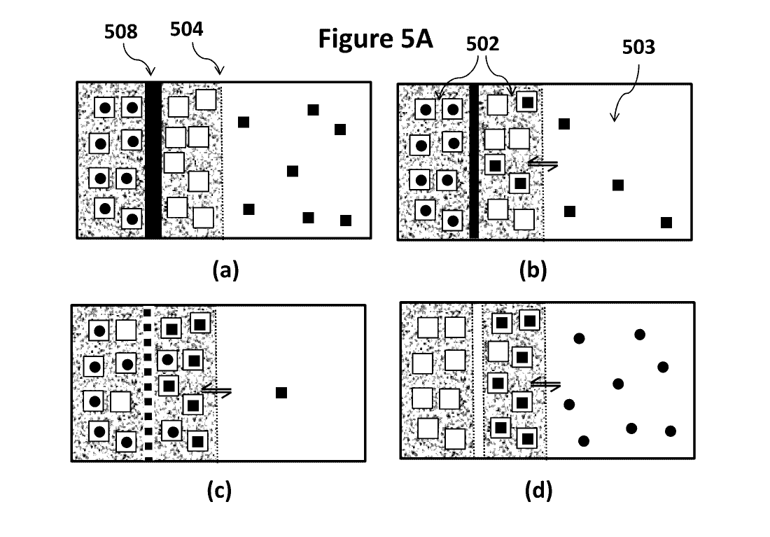

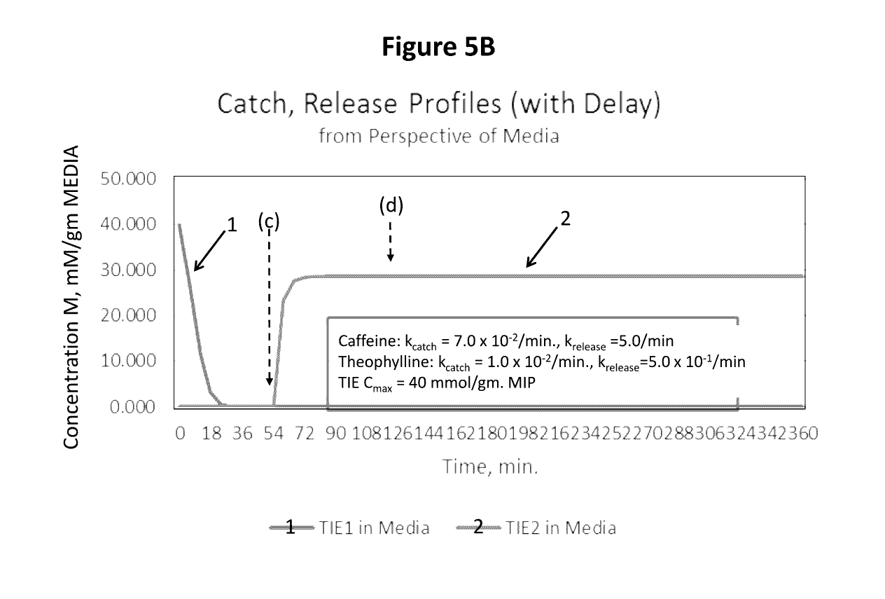

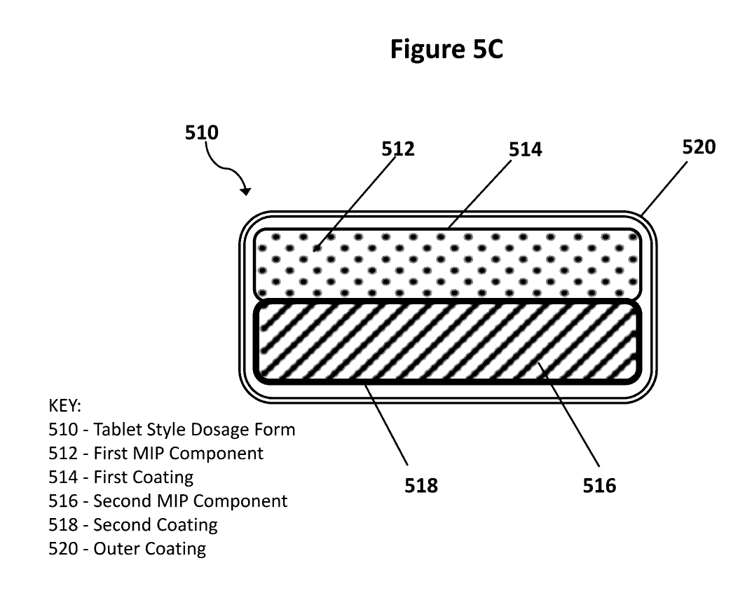

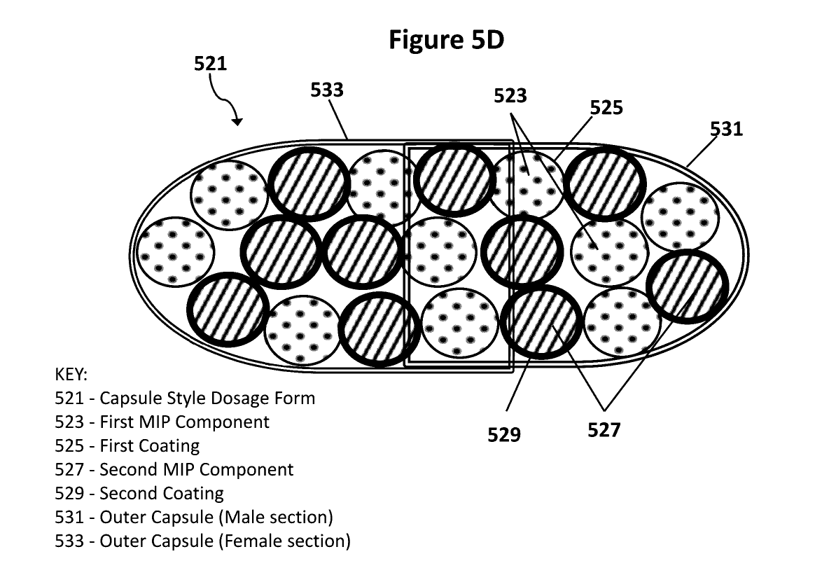

FIG. 5A shows a graphical illustration of a MIP system having an approximate equal number of two different sets of material binding sites (both shown by empty squares in frame a) selected to have significantly different k values for two different materials. One set of material binding site exhibits k values of 7.0×10⁻²/min and 5.0/min for a caffeine molecule; and a second set of material binding sites exhibits k values of 1.0×10⁻²/min and 5.0×10-2/min for a theophylline molecule, where the MIP system has a total material capacity of 40 mM/gm with respect to caffeine. Frame (a), shown initially at time zero (frame a), shows the MIP system having two components with approximately equal amounts of material, a first MIP component preloaded with theophylline molecules occupying a substantial majority of MIP binding sites and then coated with a barrier material illustrated as a solid black line on the left side of frame a. Also shown in frame (a) is a second MIP component that is not preloaded with any material and which is free of any barrier material. Both MIP components are otherwise identical in nature and both are in complete contact with a surrounding fluid media, represented in the right side of each frame. Frame (a) illustrates the state of the system at time zero, immediately prior to contacting the fluid media that contains caffeine molecules present at an initial concentration corresponding to a total of about 40 mM of free caffeine. Frames (b)-(d) show illustrative times after the MIP system is contacted with the fluid media. In frame b, the barrier material is starting to dissolve, while in frame c the barrier material has been substantially breached allowing exposure of the first MIP component to the liquid media. Frame d represents a time (Tf) at which the system substantially reaches equilibrium. Here, theophylline molecules are represented as black circles (dots), while caffeine molecules are represented as black squares.

FIG. 5B shows a graph corresponding to the illustration of FIG. 5A (frames a-d) of the concentration in the fluid media of two materials after the media has been contacted with 1 g of the example MIP system as a function of time from T=0 to T=360 min, where trace 1 shows the concentration of caffeine and trace 2 shows the respective concentration of theophylline in the media, while the vertical dashed lines denoted as (c) and (d) approximately correspond to the time frames illustrated in FIG. 5A in frames (c) and (d), respectively.

FIG. 5C shows a diagram corresponding to a cross-sectional view of an oral dosage form employing a first MIP matrix component and a coated second MIP matrix component in a dual layered tablet form, with an optional outer coating or shell surrounding the two component layers.

FIG. 5D shows a diagram corresponding to a cross-sectional view of an another oral dosage from employing a first MIP matrix component and a second coated MIP matrix component, both in the form of essentially spherical beads, contained within a lozenge shaped two part friction-fitting delivery capsule. The beads are not necessarily drawn to scale.

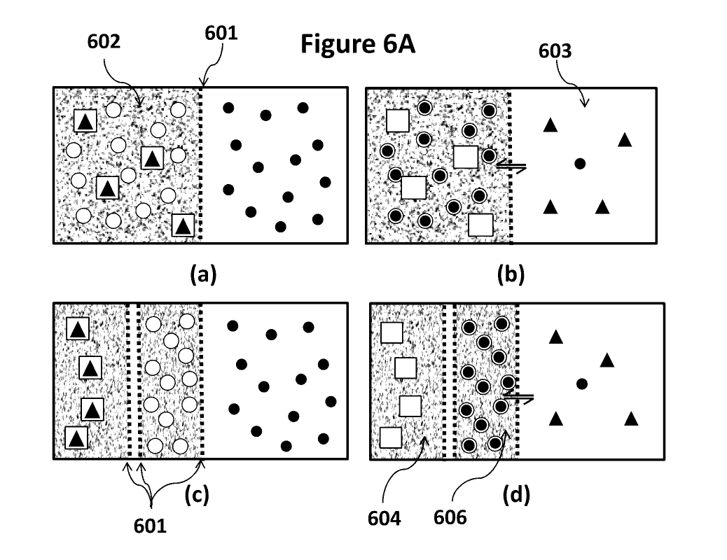

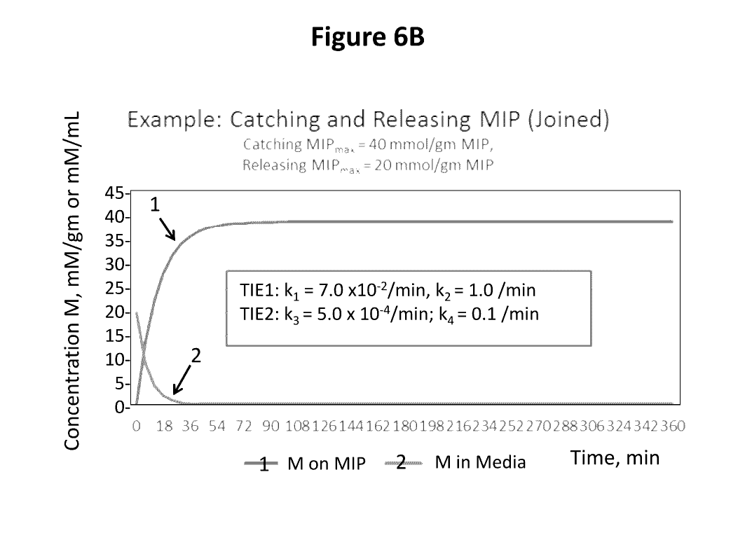

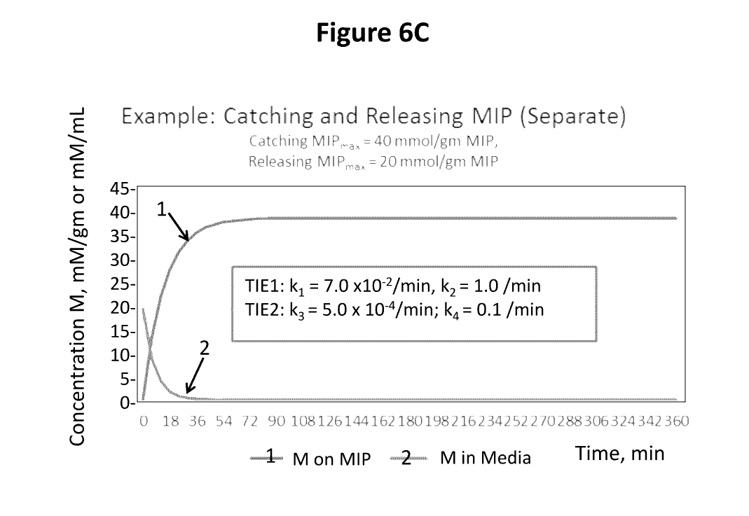

FIG. 6A shows a graphical illustration of a MIP system having a dissimilar number of two different sets of material binding sites, a first set of sites shown by empty white squares and a second set of sites represented by empty white circles. In frames (a) and (b) the two different sets of sites are present within the same MIP polymer matrix, while in frames (c) and (d) there are two physically separate MIP polymer matrices, each separate MIP matrix having only one type of site, as illustrated. Frames (a) and (c) represent the starting condition at time T=0, while frames (b) and (d) represent approximate equilibrium conditions at a final time, T=360 min, for the respective examples. In all frames, all MIP polymer matrix surfaces represented by a dotted interface (line 601) are all simultaneously in contact with the surrounding fluid media.

FIG. 6B shows a graph corresponding to the illustration of FIG. 6A (frames a and b) of the MIP concentration of two materials after the media has been contacted with 1 g of the exampled mixed MIP system as a function of time from T=0 to T=360 min, where trace 1 shows the concentration within the MIP matrix of a first material corresponding to the filled black circles, and trace 2 shows the concentration within the same MIP matrix of a second material corresponding to the filled black triangles indicated in FIG. 6A.

FIG. 6C shows a graph corresponding to the illustration of FIG. 6A (frames c and d) of the MIP concentration of two materials after the media has been contacted with 0.5 g of the each of the two example separate MIP systems as a function of time from T=0 to T=360 min, where trace 1 shows the concentration within the MIP matrix of a first material corresponding to the filled black circles, and trace 2 shows the concentration within the MIP matrix of a second material corresponding to the filled black triangles indicated in FIG. 6A.

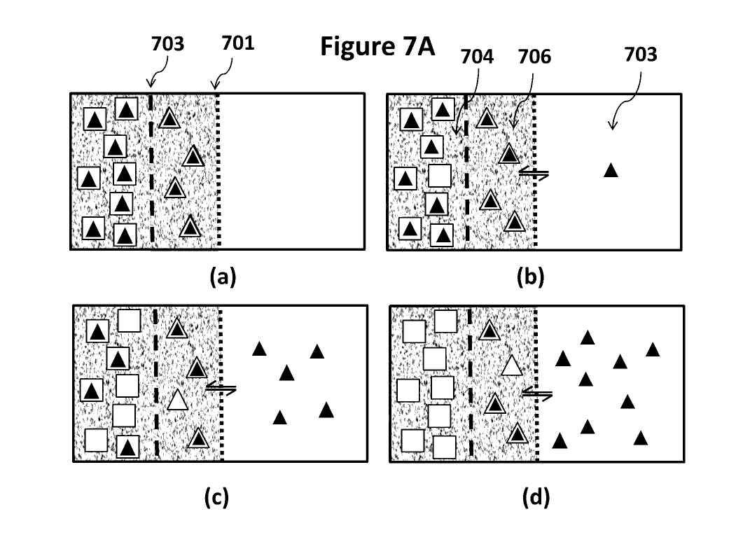

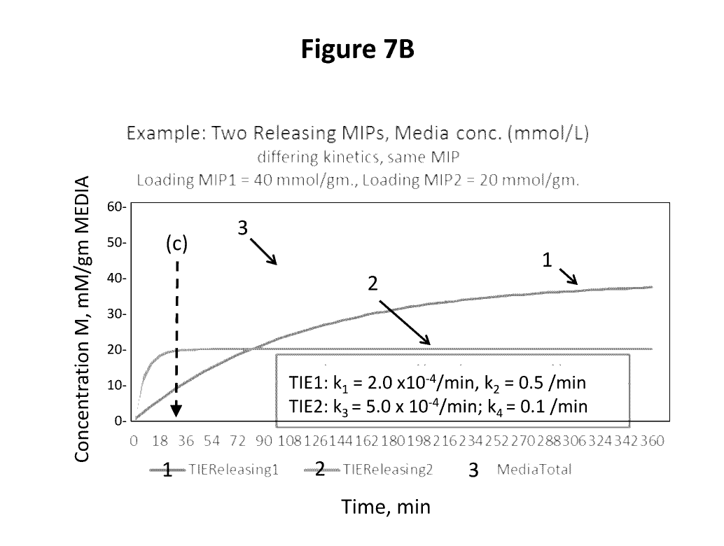

FIG. 7A shows a graphical illustration of a MIP system having two different sets of material binding sites, a first set of sites shown by empty white squares and a second set of sites represented by empty white circles. The MIP polymer matrix surface represented by a dotted interface (line 701) is in contact with the surrounding fluid media. Optionally, the MIP system can feature a dual set of material binding sites, or be two separate MIP matrices provided that both are in contact with the surrounding fluid media simultaneously represented by the slashed interface (line 703).

FIG. 7B shows a graph corresponding to the illustration of FIG. 7A (frames a-d) of the media concentration of the single material after the media has been contacted with 1 g of the exampled mixed MIP system as a function of time from T=0 to T=360 min, where trace 3 shows the total media concentration of the material, and trace 1 and trace 2 show the relative contribution to the media concentration of material released from the respective MIP sites as a function of time.

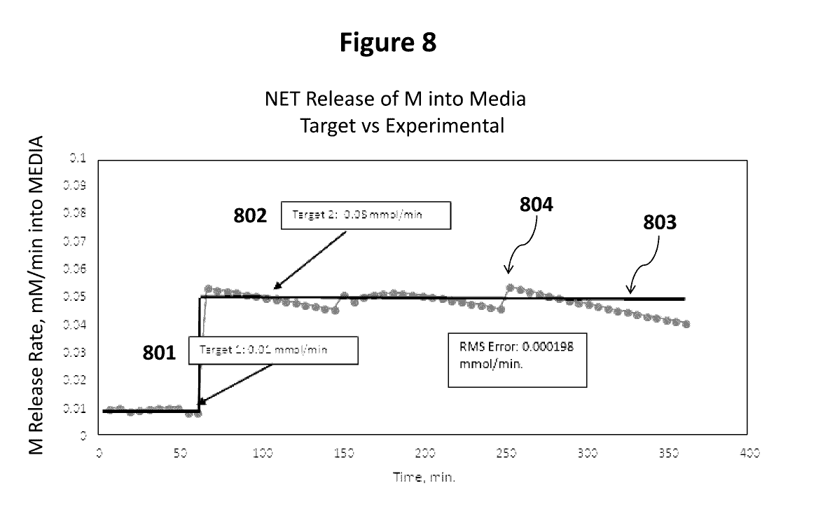

FIG. 8 shows a graph of a selected “step up” release profile from an initial to a final release rate for a material into a fluid media and the corresponding calculated release kinetics for an novel MIP system incorporating a delay release functionality.

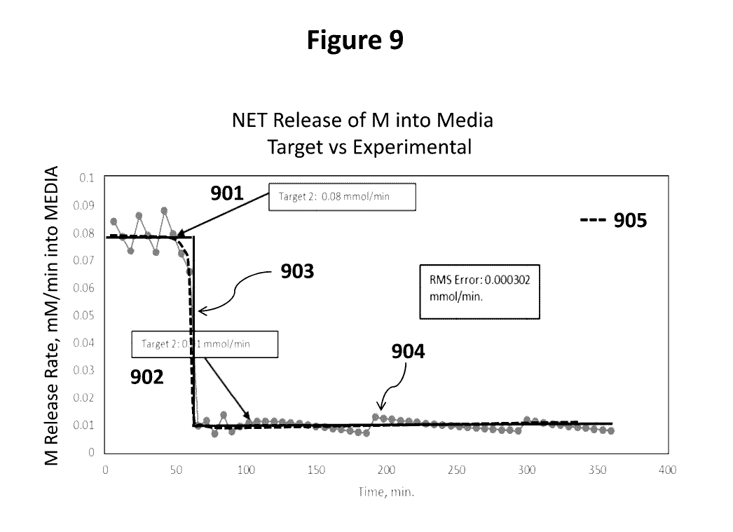

FIG. 9 shows a graph of a selected initial high dosage steady-state release, followed by a step down to a subsequent delayed low dosage steady-state release profile for a material into a fluid media and the corresponding calculated release kinetics for an novel MIP system incorporating a delay release step down functionality.

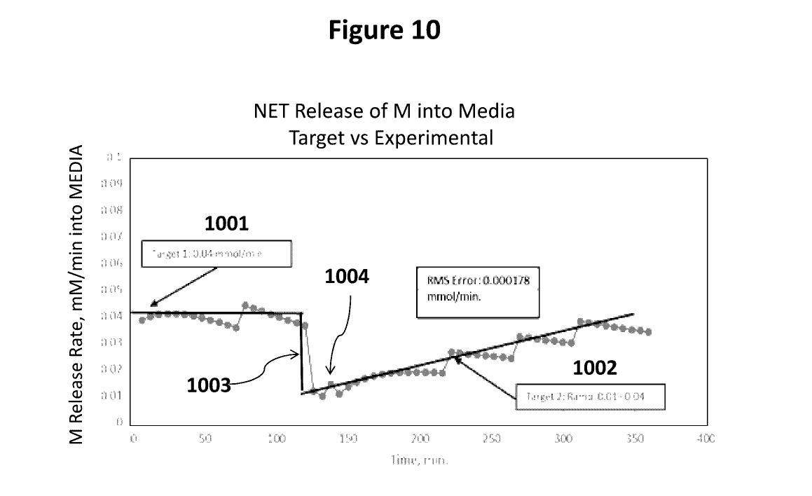

FIG. 10 shows a graph of a selected initial steady state dosage release followed by a drop to a delayed low-to-high ramp increasing release dosage profile for a material into a fluid media and the corresponding calculated release kinetics for an novel MIP system incorporating a delay release ramp-up functionality.

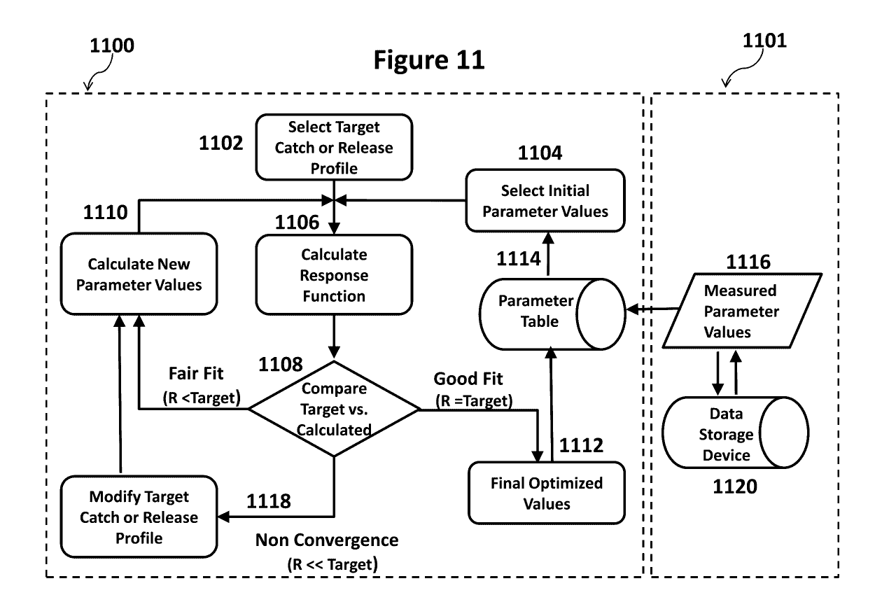

FIG. 11 shows one embodiment of an novel schematic process in diagrammatic form detailing the process for determining optimized parameter values for an novel MIP system starting with a select target catch and/or release profile seeded with initial MIP matrix parameters and system parameters derived from a database of measured or experimental parameter values, followed by successive iterative calculation steps solving for a match between desired and delivered adsorption and/or release profiles for one or more target materials, iterative calculations continued until an optimized set of target values are derived within a desired R-square fitting tolerance, with respect to the desired profile.

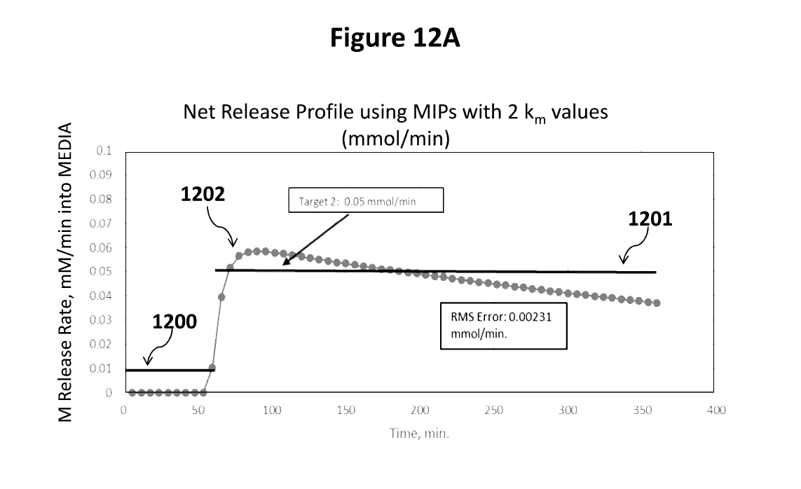

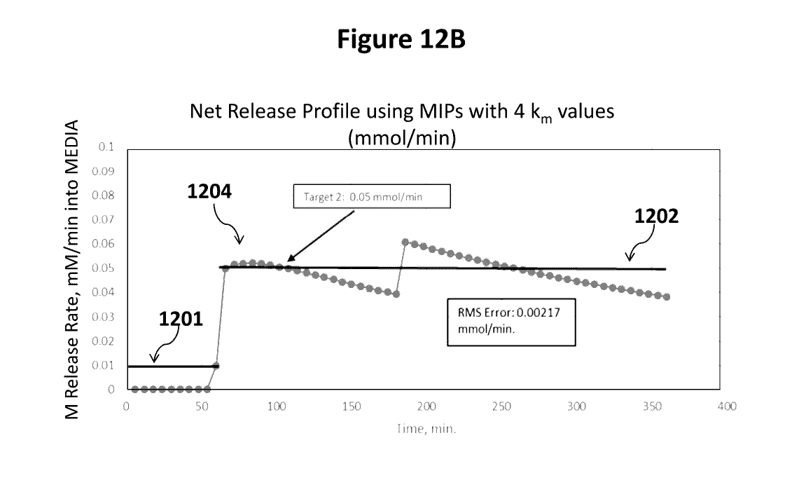

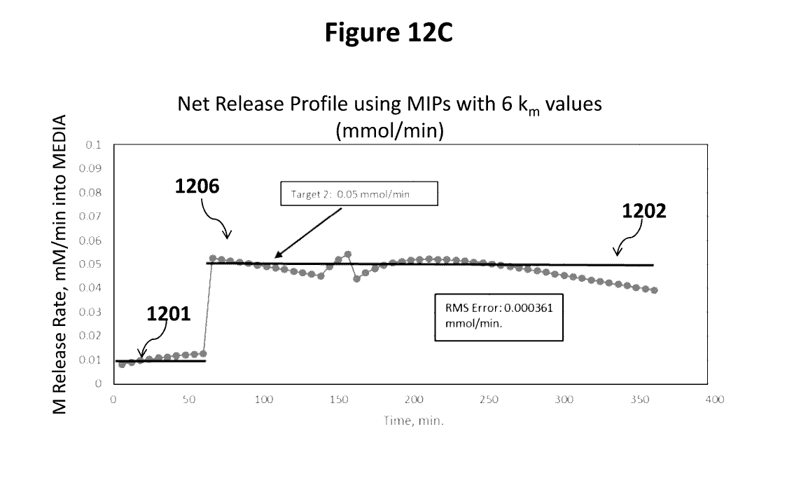

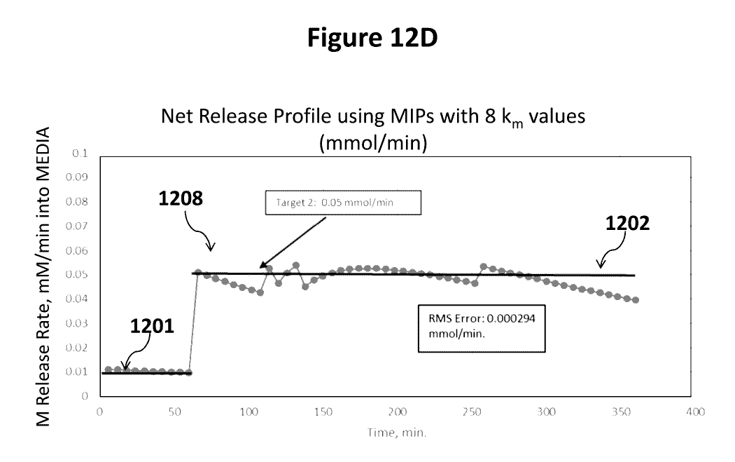

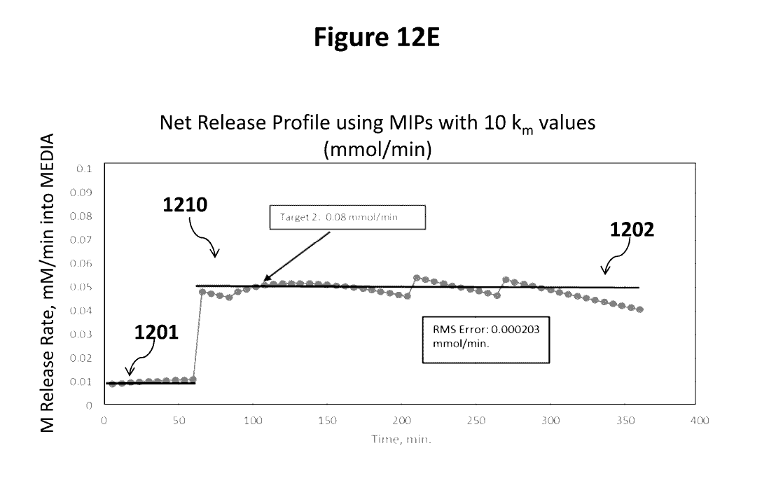

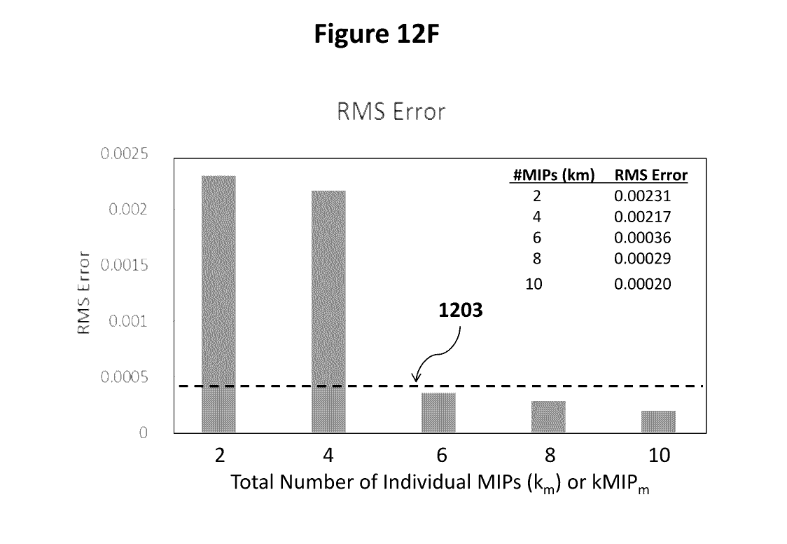

FIGS. 12A-E show the results of modeling an novel MIP system in order to achieve a desired controlled, time-delay dosage profile for theophylline with a delayed-step up release dosage capability, where an initial target release rate followed by a step-up to a higher target release rate, using MIPs having a varying number of sets of average associative binding constants.

FIG. 12F shows the root-mean-square (RMS) error of the successive novel MIP systems shown in FIGS. 12A-E compared to the desired dosage profile.

### DESCRIPTION

### Generality of Disclosure

This application should be read in the most general possible form. This includes, without limitation, the following:

References to specific techniques include alternative and more general techniques, especially when discussing aspects of the disclosure, or how the disclosure might be made or used.

References to “preferred” techniques generally mean that the inventor contemplates using those techniques, and thinks they are best for the intended application. This does not exclude other techniques for the disclosure, and does not mean that those techniques are necessarily essential or would be preferred in all circumstances.

References to a “MIP matrix” or “MIP matrices” generally mean a molecular imprinted polymer (MIP) in the physical form of a solid, particle, film, coating, web, fiber, foam, and the like and combinations thereof, wherein the physical form enables the MIP to be in fluidic contact with and capable of exchanging one or more materials with a fluid media.

References to a “MIP system” generally mean a collection or plurality of individual MIPs and/or MIP matrices combined in any desired physical form enabling each MIP or MIP matrix to be in fluidic contact with a fluid media, which is also in fluidic contact with every other MIP or MIP matrix within the MIP system, so that the ensemble is in fluidic contact with and capable of exchanging one or more materials with that fluid media.

References to a “target imprintable entity” (TIE) generally refer to a material that is capable of being molecularly imprinted and is used as a templating material to form a plurality of binding sites within a MIP matrix exhibiting an average associative binding constant for that particular TIE of k_{TIE}, and exhibiting a plurality of unique average associative binding constants, kₘ, for a set of selected n on-TIE materials.

References to “significantly” different, lower, greater, smaller, larger, etc. refer to the comparison of the (absolute) values of two numbers (A vs. B), or the values corresponding to the average values of two sets of numbers (A vs. B), in which the respective values are significantly different if numerically different by at least one significant digit within the range of the average experimental accuracy (error) for the two numbers; or if statistically distinct by at least one Least Significant Difference (LSD) unit, as determined at the 90% confidence interval for the average or median value of the averages of the two sets of numbers, respectively.

References to “suboptimal” or “suboptimum” refer to an average value of any of an association constant, binding constant, dissociation constant, equilibrium constant, exchange constant and the like, in which the absolute value of the indicated constant is lower than the absolute value of a referenced constant to which it is being compared.

References to a “catching MIP” and “catching kinetics” generally means the characteristic of a MIP with binding sites exhibiting one or more average associative binding constants of a selected non-TIE material with respect to a MIP site in which the kₘ or kₘ₍ᵣ₎ values are significantly lower than the corresponding (reverse) k_{TIE} value, so as to enable a controlled rate of adsorption (“catching”) of the selected non-TIE material into a MIP matrix or MIP system from a fluid media to achieve either a quantitative net adsorption of the non-TIE material, or enabling the establishment of a controlled equilibrium distribution of the non-TIE material between the MIP and the fluid media.

References to a “releasing MIP” and “release kinetics” generally means the characteristic of a MIP with binding sites exhibiting one or more average associative binding constants of a selected non-TIE material with respect to a MIP site in which the kₘ or k_{m(f)} values are substantially lower than the corresponding (reverse) knE value, typically by at least a factor of two, so as to enable a controlled rate of desorption (“release”) of the selected non-TIE material from a MIP matrix or MIP system into a fluid media to achieve either a quantitative net release of the non-TIE material, or enabling the establishment of a controlled equilibrium distribution of the non-TIE material between the MIP and the fluid media. It is to be noted that such classification of a binding site as a “catching” or “releasing” site is only descriptive in describing its relative average associative binding constant with respect to some other standard binding constant or reference material's binding constant under the same or similar circumstances and environmental conditions.

References to “molar” (M) or “millimolar” (mM) and respect rates including mM/sec (millimolar per second), mM/min (millimolar per minute), mM/hr (millimolar per hour) or mM/day (millimolar per day) refer to the average release and/or adsorption rate of the referenced material, expressed in molar quantities as defined by the average or aggregate molecular weight of the referenced material, absorbed into or released (desorbed) from, an novel MIP in contact with a fluid media.

References to reasons for using particular techniques do not preclude other reasons or techniques, even if completely contrary, where circumstances would indicate that the stated reasons or techniques are not as applicable.

Furthermore, the disclosure is in no way limited to the specifics of any particular embodiments and examples disclosed herein. Many other variations are possible which remain within the content, scope and spirit of the disclosure, and these variations would become clear to those skilled in the art after perusal of this application. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Read this application with the following terms and phrases in their most general form. The general meaning of each of these terms or phrases is illustrative, not in any way limiting.

### DETAILED DESCRIPTION

Conventionally, molecularly imprinted polymers (MIPs) are formed around a target imprintable entity (TIE) that is capable of being imprinted within the molecular framework of the polymer when the polymer is formed into a three-dimensional matrix hosting a plurality of the selected TIE materials within corresponding binding sites that are thus configured and tailored with respect to those TIEs. The TIE materials are then later extracted from the MIP matrix, leaving behind a plurality of cavities or sites that the TIE materials had previously occupied during the polymerization process. Without being bound by theory, it is believed that during the polymerization process, that the resulting polymeric structure configures itself physically around the TIEs present and thermodynamically adopts a structure with favorable energetic and entropic factors, thus forming sites configured to match the chemical and physical characteristics, including three dimensional features of the guest TIEs. Accordingly, these sites have a strong affinity for the TIEs, by analogy similar to that of a lock and key, the lock being the final polymer matrix and the key being the TIE, resulting in extremely high associative binding affinities of such MIP matrices for that particular TIE material.

The role of a porogen, that being the terminology used for a material that has the principal role of increasing the porosity of the resulting MIP matrix, is important in the consideration of solvent and polymerization systems employed to solubilize the TIEs and pre-polymer components (monomer, shorter polymers, cross-linking compounds, polymerization initiators and inhibitors, etc.). The selected porogen(s) and solvent(s) employed also effect the solution dynamics and chemical activities of all the chemical species present during the polymerization process, as well as to ensure homogeneity in the system prior to polymerization of the polymers (and optional copolymers) to form the MIP matrices of the present disclosure. Suitable porogens may be selected form solvents, co-solvents, wetting agents, dispersing agents, coupling agents, solubility enhancers, and other suitable materials, and combinations thereof, that act to increase the porosity of the resulting MIP matrices; increase the wettability of the pores; and/or decrease the contact angle between the MIP polymer and the fluid media used during polymerization or the desired fluid media in which the resulting novel MIP matrices are to be employed; or subsequently aid in the association of a selected material with the plurality of pores or binding sites within the MIP matrices. The term porogen is used frequently in the art, providing some insight into their nature of enhancing the formation of the pores or cavities formed around the TIEs during the polymerization process. Without being bound by theory, it is believed that the TIE sites formed are pore-like in nature, having been formed with a plurality of nearby TIEs present owing to the typical high concentrations employed, so that each pore is host to a multiple number of TIEs within a solvent or solvent-porogen cage, and following polymerization, the resulting pore is then physically defined and locked configurationally, rendering it and similar pores capable of later binding (after subsequent extraction of the TIE template material) a multiple number of TIEs or similar entities, possibly dozens or even hundreds, depending on the concentration of TIEs employed, the porogen selected, the solvent used, the polymer chemistry employed, and the polymerization conditions used to form the resulting MIP matrix.

Thus, the typical approach to producing MIPs is to select a porogen, a solvent and a polymer system so as to maximize the associative nature and selectivity of the resulting MIP matrix to exhibit TIE binding sites with extremely high specificity and high affinity for the TIEs. The high affinity results in correspondingly large associative binding constants. Further, the MIPs sites will also tend to exhibit much lower affinity or even no affinity for other materials present. Thus, a MIP polymer matrix initially formed to imprint a specific TIE, will later, when exposed to solution containing a mixture of those TIEs with other materials present, will tend to selectively adsorb the TIEs only, leaving the other materials behind in the solution. Generally, this approach is preferred where one desires to have high specificity and high associative binding constants in order to extract a desired TIE from a solution containing other unwanted materials, even those having similar structural and chemical features or characteristics.

In embodiments of the disclosure relating to the controlled release of a selected material, the MIP matrix would initially be in a state wherein most or all of the available TIE binding sites have been filled with the selected material (not necessarily the same material as the TIE used to imprint and form the binding sites), thus having a material concentration within the MIP essentially equal to the MIP matrix's saturation point. Accordingly, there would be few, if any, open binding sites at this initial stage, so that only consideration of the forward kinetics of release would be required to adequately describe the initial behavior of the system, because the reverse kinetics of adsorption would initially be inconsequential because of the low number of available, empty binding sites, regardless of the magnitude of the reverse binding (association) rate. Further, the magnitude of the reverse binding (adsorption) rate, for an overall controlled-release MIP, would be much lower in magnitude than the release rate, as overall release is the functionality that would be preferentially desired for a “releasing” system. Thus, for purposes of calculation and modeling of the novel MIP systems, the forward dynamic kₘ value is a reasonable rate constant to use to approximate the dynamic kinetic behavior, rather than K_{eq} of a novel controlled “release” system.

In the alternative, for the embodiments of the disclosure relating to the controlled adsorption of a selected material into a MIP matrix patterned with a TIE, the initial state would have most if not nearly all of the available binding sites empty and available for adsorbing the selected material. Accordingly, in this situation, only consideration of the reverse kinetics of binding would be necessary to describe the behavior of the system, and further, the forward kinetics of release would initially be inconsequential because of the low number of filled binding sites, regardless of the magnitude of the forward release (disassociation) rate. Further, the magnitude of the forward release (disassociation) rate (k_{m(f)}), for a overall controlled adsorption-type novel MIP, would be much lower in magnitude than the adsorption rate for the selected material, as controlled adsorption is the functionality that would be preferentially desired for a ‘catching’ system. Thus, for purposes of calculation and modeling of the novel MIP systems, the reverse dynamic kₘ₍ᵣ₎ value is a reasonable rate constant to use to approximate the dynamic kinetic behavior, rather than K_{eq} of an novel controlled “catch” system.

For both overall controlled “release” and controlled “catch” systems of the present disclosure, the MIPs would be designed to have one or more kₘ values (kₘ₍ᵣ) or k_{m(f)}, respectively) of sufficient magnitude to ensure the effective respective release or adsorption of the selected material, so that even at intermediate times while the systems are moving toward an equilibrium state, the same respective forward or reverse association rate constants would still effectively be representative of the system's behavior, particularly where the forward and reverse (association and disassociation) rates within a single MIP matrix differ in magnitude by a significant factor, such as at least a factor of 2 or more.

Accordingly, in further approaches and embodiments presented herein, the average associative rate constants (kₘ, m=1, . . . ) can be used to calculate, describe and model the dynamic and equilibrium states of the novel MIP matrices and MIP systems contemplated herein in relation to a fluidic media in which the novel MIP polymers are in communication.

To enable the design and selection of the appropriate MIPs polymer, matrices and systems of the present disclosure, the following mathematical discussion is presented to describe the dynamic and equilibrium characteristics of a model MIP polymer imprinted with a selected TIE material, with respect to the model MIP polymer's properties with relation to a second selected material whose media concentration is desired to be controlled in some desired and predetermined means.

Accordingly, the relationship between a MIP and a TIE (or any selected material) can be written as:

MIPₒₚₑₙ+TIE⇄MIP_{occupied} (Eq. 1)

A pseudo-reaction equation can be written as:

C_{MIP}_{ₒₚₑₙ}+C_{TIE}*⇄C_{MIP}_{_{w/TIE}} (Eq. 2)
wherein C_{TIE}* is the concentration of TIEs in the media (assumed to be constant.)

Thus, the equilibrium expression can be written as:

$${K}_{eq} = \frac{[ {C}_{{MIP}_{w \text{/} TIE}} ]}{[ {C}_{{MIP}_{open}} ] [ {C}_{TIE}^{*} ]} \qquad ( Eq . 3 )$$

Or,

$${K}_{eq .}^{*} = \frac{[ {C}_{{MIP}_{w \text{/} TIE}} ]}{[ {C}_{{MIP}_{open}} ]} \qquad ( Eq . 4 )$$

Before relating the equilibrium to the rate equations, we will need to develop a couple of additional relations. There is a relationship between the two concentrations, as:

C_{MIP}_{_{Max}}=C_{MIP}_{ₒₚₑₙ}+C_{MIP}_{_{w/TIE}} (Eq. 5)

Dividing by C_{MIP}_{ₘₐₓ} yields:

$$1 = \frac{{C}_{{MIP}_{open}}}{{C}_{{MIP}_{Max}}} + \frac{{C}_{MIPw \text{/} TIE}}{{C}_{{MIP}_{Max}}} \qquad ( Eq . 6 )$$

Defining the ratio of occupied sites to total sites available as “x” yields:

$$1 = x + \frac{{C}_{{MIP}_{open}}}{{C}_{{MIP}_{Max}}} \qquad ( Eq . 7 a )$$

Or, alternatively expressed as:

$$\frac{{C}_{{MIP}_{open}}}{{C}_{{MIP}_{Max}}} = 1 - x \qquad ( Eq . 7 b )$$

We are now in a position to relate the equilibrium K to the rate constants, k_{association} and k_{dissociation}, and the concentrations, C_{MIP}_{ₒₚₑₙ} and C_{MIP}_{_{w/TIE}}, noting that the expression denoted “association” is the same as “catch” (adsorption), and “dissociation” is the same as “release” (desorption).

The equilibrium equation can then be written as:

$${K}_{eq} = \frac{[ {C}_{{MIP}_{w \text{/} TIE}} ]}{[ {C}_{{MIP}_{open}} ] * [ {C}_{TIE}^{*} ]} = \frac{{k}_{association}}{{k}_{dissociation}} \qquad ( Eq . 8 )$$

For our purposes, we will assume a first-order rate relationship, expressed as:

$$\frac{d [ x ]}{d t} = {k}_{association} * [ x ] \qquad ( Eq . 9 )$$

And correspondingly, for the dissociation:

$$\frac{d [ 1 - x ]}{d t} = {k}_{dissociation} * [ 1 - x ] \qquad ( Eq . 10 )$$

Solving these two equations and returning to the concentration terms (instead of the fraction terms), then yield an expression for the rate of association, which is given by:

C_{MIP}_{ₜ}=C_{MIP}_{_{Max}}(1−e^{-k}^{_{association}}^{t}) (Eq. 11)

Thus, the corresponding rate of dissociation is then given by:

C_{MIP}_{ₜ}=C_{MIP}_{ₘₐₓ}e^{-k}^{_{dissociation}}^{t} (Eq. 12)

The present disclosure also encompasses MIP systems that have been formed with a plurality of modified material binding sites (MIPₘ) that exhibit at least one associative binding constant (k_{MIPm}, m=1) that is significantly lower than that exhibited by a material interacting with an unmodified TIE site (MIPᵤ) with respect to the TIE material used in the formation of the MIP matrix, such that:

k_{MIPm}<<k_{MIPu} (Eq. 13)
wherein the associative binding constants denoted by “k” refer to the average value of the collective binding constants of all similar MIP sites for a particular material, which typically manifest as a mono-modal and fairly narrow Gaussian average as site-to-site variations in molecularly imprinted polymer systems are fairly small owing to the manner in which the TIE(s) are imprinted, producing some uniformity in binding characteristics across the multitude of sites formed during MIP preparing.

Further, the present disclosure also encompasses MIP systems that feature a plurality of modified material binding sites (2, 3, . . . p) such that the collective plurality of associative binding constants is selected from the set of significantly different or modified TIE sites having unique associative binding constants that are all significantly different from each other and collectively are also significantly lower than that exhibited by an unmodified TIE site with respect to a selected material, expressed in set notation below such that:

{k_{MIP}|k_{MIPm}ε(k_{MIP1}<<k_{MIP2}<<k_{MIP3} . . . <<k_{MIPp}),k_{MIPm}<<k_{MIPu},m=1,2, . . . p} (Eq. 14)
wherein the mathematical expression, “a<<b” or “significantly less than”, denotes that the value of a is at least statistically less than the value of b, and wherein the set expression “{k_{MIP}|k_{MIPm}ε . . . }” denotes that all values of k_{MIP} are selected from a set of k_{MIPm} values that are all significantly different from each other and simultaneously, less than and significantly different then the value of k for a MIP system formed using an unmodified TIE material that exhibits an average associative binding constant of k_{MIPu}

Thus, in contrast to a MIP system employing an unmodified TIE for TIE site formation and thus exhibiting an average associative binding constant of K_{MIPu}, the novel MIPs exhibit at least one associative binding constant for a material that is significantly less than the average associative binding constant of an unmodified TIE site. Surprisingly, it has been discovered that when such a programmed MIP system having one or more associative binding constants is employed, that the MIP matrix has utility in controlling the binding characteristics and rates of both the capture and release of both unmodified TIEs and TIE-like materials alike, enabling pseudo zero- and first-order capture and release kinetics to be achieved, as well as programmable MIP systems capable of generating and maintaining an equilibrium distribution of one or more TIE and TIE-like materials between the MIP system and a fluid media in contact with the novel MIPs.

Accordingly, the present disclosure offers a unique approach for the programmed and controlled uptake and release of TIEs and TIE-like materials, the latter being materials that are chemically, physically and with respect to their associative binding characteristics, similar to, but not identical to the unmodified TIE materials used to produce the imprinted polymer binding sites. Examples, may include, but are not limited to TIE isomers, homologues, chemically modified TIEs and structural as well as stereo isomers of the unmodified TIE, as well as materials that share at least one similar chemical group, substituent, or unique chemical or physical feature with that of the unmodified TIE material.

Further, it has been surprisingly discovered that when one or more MIP matrices having a plurality of modified binding sites are combined exhibiting at least two significantly different associative binding constants for a selected material, then controlled catch and/or release capabilities providing pseudo-linear and zero-order ramp catch and/or release kinetics are exhibited by the novel MIP systems, as well as operating to achieve and maintain an equilibrium distribution of a material between the MIP systems and a fluid media in contact with the novel MIPs.

In addition, it has been discovered that when one or more of the novel MIP systems are combined with a simple delay functionality, being a means to delay exposure of the MIPs to the fluid media and including for example, but not limited to, a time-delay coating or sacrificial barrier, that the novel MIP systems can provide additional delayed catch and release behaviors, as well as delayed ramp and step-function-like catch and release profiles that cannot be achieved with conventional MIP systems.

### Programmable Catch and Release MIP Systems

In one embodiment of the present disclosure, a MIP system employs a TIE for its formation, that then exhibits a modified associative binding constant with respect to a material selected from the TIE, a TIE-like analog, and combinations thereof. In a first example, the system we are envisioning will provide a MIP structure that releases selected materials, per the first-generation models, and also a MIP structure that catches a second set of selected materials. Multiple combinations of these two features will be presented with respect to an ideal “zero order” kinetics solution, to determine the characteristics of the MIP materials required in order to accomplish that task.

In one embodiment, there is a selected plurality of MIPs with an average high association binding affinity that will operate as “catching” MIPs, which in general terms can be viewed as being MIPs with binding sites much more efficient at binding the selected material then a second selected plurality of “releasing” MIPs, the latter generally having binding sites with lower associative binding affinities than the “catching” MIPs. In addition to the ‘catching’ MIPs having a higher average associative binding affinity for a material, these “catching” MIPs are also likely to be much faster than the “releasing” MIPs in taking up the desired material from a media, as the higher average associative binding affinity favors a material bound to a catching MIP as opposed to a free material in the media or a material bound to a less receptive (lower binding affinity) ‘release’ MIP site. Thus in operation, as soon as a material is released from its binding site in the “releasing” MIP, it is quickly and efficient “taken up” by one of the plurality of “catching” MIP binding sites. Accordingly, be combining at least two MIPs having significantly different average associative affinities, one can tailor the resulting catch and/or release kinetics of either an absorbed material present in one of the MIPs, or that material present in a fluid in contact with the MIPs systems.

For modeling to be successful, it should account for the collective behavior of the MIPs, addressing which MIP(s) take up that released material, and, if many materials are released, in what proportion. A second consideration is that the “catching” MIPs will likely not be able to ‘satisfy’ all of its capacity to “catch” all available materials, because there will be a shortage of released materials.

Further, where a first “catching” MIPs average associative binding affinity is close, even if significantly different than that of a second “releasing” MIP, this will result in the former catching available materials at about the same rate as the “releasing” MIP releases them. Thus, the instantaneous bulk concentration of the material in the MIPs and fluid system will be driven by the ratio of capacities between the “releasing” MIPs and the “catching” MIPs for that particular material.

Where the “catching” MIP catches much slower than the “releasing” MIP, then the kinetics of the “catching” MIP should solely be driven by the “catching” kinetics, providing that the latter associative binding affinity is greater than the catching MIPs binding affinity, since it may be the rate-limiting reagent in the system.

Further, for very dilute solutions of available materials in the fluid media present with the MIPs, or circumstances where the bulk solution is large (i.e. there are few available excess of materials available for capture relative to the amount released), then the “catching” MIP will be limited, because it cannot catch unless and until it finds an available material. For these types of systems, the bulk concentration of materials will not be a consideration.

Finally, because most of the example models of interest to be presented for controlling solution concentration of MIPs in a fluid media involve the first release of materials from a saturated MIP host, then the selected associative binding affinities of interest are those in which the “catching” MIPs act faster than the “releasing” MIPs, and thus by virtue of the catching MIPs having the higher average associative binding constants, one can focus on kinetics driven by the concentration of materials on the respective MIPs, rather than the bulk material concentrations in the contacting fluid media. Naturally, further examples and embodiments are within the scope of the present disclosure wherein the kinetic profiles are reversed, and the bulk material concentrations in the fluid media are best used for modeling purposes.

Three main factors may contribute to the overall rate of catching a desired molecule (the “material”), and apply individually to each of the modeled catching MIPs sites: (a) the association kinetics (adsorption/desorption) of each individual catching MIP site; (b) the extent of loading (degree of occupancy of each individual MIP site; and (c) the availability of materials to catch (i.e., free, unassociated materials in the fluid media).

Now, for a system or collection of MIPs sites, one can designate the total number of “releasing” sites to be represented by N, while the total number of “catching” sites be represented by M. Now, if there is an excess of materials available in a liquid media or solution in intimate contact with the MIP polymer bearing a plurality of each type of MIP binding site, and each MIP site starts out in time as being completely empty; and each MIP site follows a 1^{st} (first) order catching or binding mechanism, that the equation (based on the concentration of materials “caught”) describing the binding kinetics is as follows:

C_{m,t}=C_{m,max}(1−e^{-k}^{ₘ}^{t}) (Eq. 15)
wherein C_{m,t} is the concentration of materials bound to catching MIPs at time T=t, and C_{m,max} is a maximum limiting value, being the maximum possible concentration of materials that can be bound to the plurality of catching MIP sites, denoted as MIPₘ; and kₘ is the rate of association for MIPₘ in units of min⁻¹ (1/min).

For any arbitrary time period, At, the amount or concentration of material entities (m) caught is then expressed as:

ΔC_{m,t}_{ᵢ}=C_{m,t}_{ᵢ}−C_{m,t}_{_{i−1}}=C_{m,max}(e^{-k}^{ₘ}^{t}^{_{i−1}}−e^{-k}^{ₘ}^{t}^{ᵢ}) (Eq. 16)
wherein tᵢ is the (i)^{th} time interval between the initial starting time, t₀ and the final or ending time period, t_{f}; and t₍ᵢ₋₁₎ denotes the (i−1)^{th} time interval.

Thus, in an unconstrained material environment, each MIPₘ would acquire a number of material entities (m) consistent with its own collective, but isolated kinetic behavior. This is a first approximation, although it is likely that there is a distribution of binding constants for the various individual binding sites, although the distribution could be fairly narrow; and there may also be second order effects due to interactions between the sites.

Nevertheless, where competition for materials occur amongst a collective plurality of available MIP sites, they compete for binding according to the relative rates of material acquisition by each individual site. Thus, they will proportionate, as:

$${r}_{m , i} = \frac{Δ {C}_{m , i}}{{\sum }_{j = 1}^{M} Δ {C}_{j , i}} \qquad ( Eq . 17 )$$

wherein r_{m,i} is the fractional distribution of materials binding to MIPₘ during the i^{th} interval of time.

However, there may be limitations. The first being that the most aggressive MIPs, i.e. those having a higher binding rate or constant amongst the collective plurality will tend to bind materials more efficiently and thus will very likely bind the materials preferentially and therefore “fill up” more quickly. A second limitation is that the binding kinetics are also likely to be somewhat slower on a partially filled MIP site compared to an empty MIP site, as well as a nearly fully filled MIP site than a partially filled MIP site, as it is conventionally known a given MIP binding site generally configures itself, based on the nature of the polymer matrix, solvent and the porogen media used during the synthetic formation and imprinting process, to bind a multiple number of materials per site. Thus, even considering an individual, isolated MIP site, the time dependent binding kinetics or constant for that site would be expected to vary somewhat with the extent of bound materials modifying, at least from a simple stochastic view anticipating some binding site competition, the expected binding constant as a function of bound material entities (m) and hence resulting in some variation in the binding constant with time. Accordingly, in some instances there may be a collection of MIPs that will not receive a full quantity of materials during a particular time interval, i, so that the MIP site's concentration at time t=i will not match 1st order binding kinetics, and that subset of MIP sites may actually succeed in acquiring more material entities during that time interval, i, than first order kinetics would predict.

However, assuming that the system will initially follow first order kinetics when the ratio of material entities (m) to available MIP sites is very high, but to only use the initial equation to determine the initial system parameters in order to determine approximate starting values and the various system parameters. Once the initial set of values and system parameters are determined to a reasonable first approximation, the catch (binding, adsorption) and release (desorption) characteristics can be refined by iterative modeling, as is commonly done for dynamic systems that exhibit some degree of time dependent behavior. Here, the initial state, approximated by calculations over a first, initial time period are used to calculate the initial binding parameters and then to more realistically approximate the number of available MIP binding sites and number of available materials, and the corresponding distribution of bound and free materials. Each successive iteration thus enables a more accurate calculation or estimate of the new values for each species concentration at the start of that incremental time period for that collection of MIPₘ and material concentration. These values are then used as the initial starting conditions for the next time interval, i+1, and iteratively, the same process used to a selected final time interval, t_{f}. Mathematically, this can be expressed as below:

ΔC_{m,t}_{ᵢ}=C_{m,t}_{ᵢ}−C_{m,t}_{_{i−1}}=Xᵢr_{m,i} (Eq. 18)
wherein for the next time interval, i+1:

C_{m,t}_{ᵢ₊₁}=C_{m,t}_{ᵢ}+Xᵢr_{m,i} (Eq. 19)
and wherein Xᵢ is the concentration in millimoles (mM) of material released by all releasing MIPs during the time interval, i.

This holds true providing that the system remains under the reasonable constraint that:

$${X}_{i} {r}_{m , i} \leq \frac{{C}_{m , i - 1} {e}^{- {k}_{m} Δ t}}{{gm}_{cMIP} * {f}_{{cMIP}_{m}}} \qquad ( Eq . 20 )$$

wherein gm_{cMIP} is the total weight in grams (gm) of catching MIPs, denoted as cMIP; and f_{cMIPm} is the fraction of catching MIPₘ's within the total weight of all catching MIPs or cMIPs.

Thus, the constraint imposed by Eq. 20 limits the binding of materials by providing that any given MIPₘ site cannot receive more materials than it would in an unconstrained kinetic environment. The binding is thus normalized so that, if the fraction of catching cMIPs is small, they cannot receive a disproportional abundance of the material entities. With this constraint (Eq. 20), the concentration as a function of time can now be expressed in the following equation:

$${C}_{m , {t}_{i + 1}} = {C}_{m , {t}_{i}} + ( {X}_{i} {r}_{m , i} or \frac{{C}_{m , i - 1} {e}^{- {k}_{m} Δ t}}{{gm}_{cMIP} * {f}_{{cMIP}_{m}}} ) \qquad ( Eq . 21 )$$

Now that we have a reasonable value for Cₘ at time interval ti, and can account for the number of materials that each MIPₘ will “catch” during that time interval, we can calculate the net or ‘excess’ number of material entities (#M) released into the system, as:

E=Excess #M=#M Released−#M Caught (Eq. 22)

Accordingly, the release is then governed by a modified version of Eq. 18, being expressible now for each successive time interval, t+i, as follows:

Xᵢ=Σₙ₌₁^{n=N}Gₜₒₜₐₗ*fₙ(C_{n,(0)}e^{-k}^{ₙ}^{t}−C_{n,(0)}e^{-k}^{ₙ}^{t+i}) (Eq. 23)
wherein Xᵢ is the concentration in mM of material entities (m) released across all MIPs for the time interval, i; Gₜₒₜₐₗ is the total number of grams of releasing MIPs present; fₙ is the fraction of releasing MIPs of type n, being denoted as MIPₙ; and C_{(n,(0)} is the starting concentration of materials already bound to MIPₙ sites, expressed in units of mM/gram MIPₙ.

Equation 23 can now be expressed as a function for any single time interval, i, as follows (and again under the constraint imposed by Eq. 20):

$$Δ {C}_{m , i} = {C}_{m , {t}_{i + 1}} - {C}_{m , {t}_{i}} = ( {X}_{i} {r}_{m , i} or \frac{{C}_{m , i - 1} {e}^{- {k}_{m} Δ t}}{{gm}_{cMIP} * {f}_{{cMIP}_{m}}} ) \qquad ( Eq . 24 )$$

Which then allows the total concentration of caught material entities (m) to be expressed as:

$${Y}_{i} = {\sum }_{m = 1}^{m = M} ( {X}_{i} {r}_{m , i} or \frac{{C}_{m , i - 1} {e}^{- {k}_{m} Δ t}}{{gm}_{cMIP} * {f}_{{cMIP}_{m}}} ) \qquad ( Eq . 25 )$$

wherein Yᵢ is the total concentration in millimoles (mM) of materials caught during the time period, i.

This derivation now allows the terms in Equation 20 to be substituted and re-expressed to show the net number of excess material entities released during the interval, i, which is as follows:

$$Γ = {\sum }_{n = 1}^{n = N} {G}_{total} {f}_{n} ( {C}_{n , ( 0 )} {e}^{- {k}_{n} t} - {C}_{n , ( 0 )} {e}^{- {k}_{n} t + i} ) - {\sum }_{m = 1}^{m = M} ( {X}_{i} {r}_{m , i} or \frac{{C}_{m , i - 1} {e}^{- {k}_{m} Δ t}}{{gm}_{cMIP} * {f}_{{cMIP}_{m}}} ) \qquad ( Eq . 26 )$$

wherein Γ (Gamma) is the net number of material entities (m) released (excess) during the interval, i, but still subject to the constraint that:

$${X}_{i} {r}_{m , i} \leq \frac{{C}_{m , i - 1} {e}^{- {k}_{m} Δ t}}{{gm}_{cMIP} {f}_{{cMIP}_{m}}} \qquad ( Eq . 27 )$$

Now that we can express the net release, Γ (Gamma), as a function of user defined inputs, we are in a position to develop the equations to design and measure performance of the MIPs systems, that is to say select and then tailor the MIPs to exhibit the desired catch and/or release profiles.

Performance is measured by iteratively calculating the model to achieve some desired (and acceptable) minimum error versus a selected target parameter. In one embodiment, the selected target parameter to be modeled could be a desired net release rate, Γ, or some minimal variation from the average release, X̅. In an earlier embodiment described herein above, the variation was based on one target for all time. In this following embodiment, we will illustrate a step change in a selected value after some time, t, to reflect an additional ‘delay’ parameter that can be introduced to account for a time-delay functionality added to one or more of the MIPs systems.

The average release over time interval J is given in Equation 28 below:

$$\overline{{X}_{J}} = ( {\sum }_{j = 1}^{j = J} {\sum }_{n = 1}^{n = N} {G}_{total} * {f}_{n} ( {C}_{n , ( 0 )} {e}^{- {k}_{n} {t}_{j}} - {C}_{n , ( 0 )} {e}^{- {k}_{n} {t}_{j + 1}} ) - {\sum }_{m = 1}^{m = M} ( {X}_{j} {r}_{m , j} or \frac{{C}_{m , j - 1} {e}^{- {k}_{m} Δ t}}{{gm}_{cMIP} * {f}_{{cMIP}_{m}}} ) ) \frac{1}{J} \\ \overline{{X}_{J}} = ( {\sum }_{j = 1}^{j = J} {\sum }_{n = 1}^{n = N} {G}_{total} * {f}_{n} ( {C}_{n , ( 0 )} {e}^{- {k}_{n} {t}_{j}} - {C}_{n , ( 0 )} {e}^{- {k}_{n} {t}_{j + 1}} ) - {\sum }_{m = 1}^{m = M} ( {X}_{j} {r}_{m , j} or \frac{{C}_{m , j - 1} {e}^{- {k}_{m} Δ t}}{{gm}_{cMIP} * {f}_{{cMIP}_{m}}} ) ) \frac{1}{J} \qquad ( Eq . 28 )$$

wherein J is the total number of time intervals, j. A simple extension of this iterative approach allows for multiple time intervals (and allowing for step changes), such that:

I=J+K (Eq. 29)
wherein I is the total of all time intervals, j and k, combined; K is the total number of time intervals in the second step, k (unitless, but selected to correspond to some convenient repeating time period).

It should be noted that Eq. 29 can easily be modified, by changing J indices to K indices, to determine the average release of materials during the total second time interval, K.

Next, one can determine an expression for the acceptable degree of error allowable for achieving a predictive value with acceptable accuracy, for example corresponding to a 90% or 95% confidence level. While determining error versus an average is possible, it is more helpful and instructive to determine error versus a pre-determined target, either for the J^{th} interval or the K^{th} interval.

The error for any given, single time interval, can be expressed as:

$${E}_{J , j} = ( {\sum }_{n = 1}^{n = N} {G}_{total} {f}_{n} ( {C}_{n , ( 0 )} {e}^{- {k}_{n} {t}_{j}} - {C}_{n , ( 0 )} {e}^{- {k}_{n} {t}_{j + 1}} ) - {\sum }_{m = 1}^{m = M} ( {X}_{j} {r}_{m , j} - {T}_{j} ) \qquad ( Eq . 30 )$$

wherein Xⱼr_{m,j} can also be expressed as:

$$( \frac{{C}_{m , j} {e}^{- {k}_{m} Δ t}}{{gm}_{cMIP} * {f}_{{cMIP}_{m}}} ) ;$$

and wherein E_{J,j} is the difference between the target release value and the actual release value corresponding to the j^{th} interval of time segment, J; Tⱼ is the target release value for segment J.

Alternatively, equation (16) can be easily modified and solved to express the error for the K^{th} time segment as well, if desired.

For a minimization routine, one generally seeks to minimize the error by minimizing the root-mean square (RMS) error, which is expressed below as:

$${E}_{rms , J} = {\sum }_{j = 1}^{j = J} ( ( {\sum }_{n = 1}^{n = N} {G}_{total} {f}_{n} ( {C}_{n , ( 0 )} {e}^{- {k}_{n} {t}_{j}} - {C}_{n , ( 0 )} {e}^{- {k}_{n} {t}_{j + 1}} ) - {\sum }_{m = 1}^{m = M} {( {X}_{j} {r}_{m , j} - {T}_{j} )}^{2} / J \qquad ( Eq . 31 )$$

wherein Xⱼr_{m,j} can also be expressed as

$$( \frac{{C}_{m , j} {e}^{- {k}_{m} Δ t}}{{gm}_{cMIP} * {f}_{{cMIP}_{m}}} ) ;$$

and wherein E_{rms,J} is a variation of the root-mean-square error of the calculated value versus the target value, Tⱼ.

Finally, the total root-mean-square error is the sum of the root-mean-square errors of both periods, J and K, which is calculated as follows:

E_{rms,TOTAL}=E_{rms,J}+E_{rms,K} (Eq. 32)

With Equation 32, one can now optimize a system for both the “catch” and “release” of materials by optimizing a plurality of collective MIP parameters by means of either using estimated or actual adsorption (catch) and desorption (release) association constants for the MIPs system with respect to the desired material.

In another embodiment, it may be desirable to also consider one additional variation: to delay the contact of a selected MIP with the media either containing the desired material to be caught or adsorbed, or into which the desired material is to be released. In one example embodiment of the present disclosure, one (or more in a plural system) of the MIPs can be coated with a suitable material that would slowly dissolve in the media, resulting in the exposure of that MIP to the media after a desired time delay has occurred following introduction of the coated MIP ensemble into the media.

In practice, a typical delay coating around a core of MIP polymer matrix would be constructed of some material that is slowly or sparingly soluble and/or disintegrates over a desired time period within the fluid or media of choice, so that it would take a period of time to be sufficiently dissolved or compromised so as to expose the core of MIP polymer to the bulk fluid or media. In a real-world system, it is likely that a coated MIP would “become active” gradually as the time-delay coating dissolves or becomes compromised, so that more and more available binding sites eventually become exposed to the bulk media (for example water, blood or other liquid), until the coating is sufficiently removed or compromised so the bulk of the available MIP sites on the MIP polymer core are active, now being totally exposed and accessible to interact with the bulk media. However, for ease in modeling and calculating a response in order to identify the desired system parameters, one can make a first approximation by assuming that the delay coating operates intact for a desired time interval, and then becomes fully dissolves or disintegrates, behaving for this approximate estimate as an “off-on” or triggered-release delay system. In an iterative approach by calculation, little error is found if the coating undergoes this transition within one time period of the iteration. In practice, this approach provides a fairly good first approximation in any event for most typical coating materials, which upon a first initial breach, act to effectively expose the majority of the protected core to the media once at least one hole, breach, fissure or infusion of media through the barrier coating material occurs.

However, assuming an “off-on” or step function change for a delay mechanism, then the fraction of available MIP sites for a second set of material entities (n) can be defined for the time interval preceding the trigger point (“off” time period) and the time interval after the trigger point, or on period in which the delay or barrier coating is no longer capable of exerting an appreciable effect on the availability of MIP sites to interact with as in the bulk media, or conversely for MIP sites preloaded with material n to begin to equilibrate and release materials into the media.

Next, the mass fraction can be defined as follows:

$${X}_{n , i} = \begin{matrix}for t \leq {T}_{n} , {X}_{n , i} = 0 \\ for t > {T}_{n} , {X}_{n , i} = {X}_{n , ( i - {T}_{n} )}\end{matrix} \qquad ( Eq . 33 )$$

wherein Tₙ is the time period at which point in time the MIPₙ coating is sufficiently compromised or removed and the MIP core begins functioning as if no coating was present. Likewise, for the “catching” MIPs, a similar approach yields:

$${X}_{m , i} = \begin{matrix}for t \leq {T}_{m} , {X}_{m , i} = 0 \\ for t > {T}_{m} , {X}_{m , i} = {X}_{m , ( i - {T}_{m} )}\end{matrix} \qquad ( Eq . 34 )$$

wherein X_{m,i} is the mass fraction of MIP sites available for catching materials present in the bulk media, and Tₘ is the time period at which point in time the MIPₘ coating is sufficiently compromised or removed and the MIP core begins functioning as if no coating was present.

Accordingly, now that the characteristic behaviors for desirable catch and release systems have been mathematically described as above, some specific example embodiments of the present disclosure can be presented.

### DETAILED EMBODIMENTS

In one embodiment of the present disclosure, a series of MIP matrices are contemplated having a range of suboptimal and significantly different average associative binding constants with respect to a selected material, which is initially present in an associated fluid media. Generally, it is the average value of the collective set of associative binding constants associated with the plurality of available binding sites within the MIP that is considered as the representative associative binding constant value for a selected material and MIP matrix, recognizing that the binding site properties tend to follow a normal statistically Gaussian distribution with respect to the set of k's and an average value, kₘ, as discussed in greater detail herein.

In one embodiment of the present disclosure, FIG. 1 shows a graph of a model release system being a combination of two MIP matrices each having a distinctive release rate with respect to a preloaded material (theophylline). Here the instantaneous concentration of this material in a fluid media is plotted as a function of time, from an initial period at t=0 to about 360 mins. A first example MIP matrix has an average dissociation or release constant of about 1.0×10⁻²/min with a loading capacity denoted by Cmax, of about 40.0 mmol/g of the first MIP (MIP 1). The second example MIP matrix has a substantially lower average dissociation rate or release rate constant of about 4.0×10⁻³/min, but also having a similar loading capacity of about 40.0 mmol/g with respect to a preloaded material. The first and second trace (numbered 1 and 2, respectively) show the individual MIP matrices characteristic release profile over time once the MIP polymers are contacted with a fluid media.

Both curves show an initial rapid increase to a starting maximum effective release rate, reflecting the high initial release from the respective matrices owing to the magnitude of the average dissociation constant combined with the initial MIP matrices having large initial concentrations (i.e. binding sites previously saturated with the material up to the loading capacity). Over time, the two release curves decrease as the effective material concentration within the respective MIP matrices decreases (naturally, the release rate constant being constant). Trace 3 shows the overall combination, or actual delivered material dosage delivery rate into the fluid media, being the result of the sum of the combined MIP matrix systems. Here it is seen that a combination of the novel MIP matrices can provide for a release profile that is tunable, by means of selecting MIP polymers that have the desired average dissociation rate constants which would provide the desired overall dosing profile for the selected material.

Mathematically, the release curve of MIP 1 (Trace 1) can be expressed by the following equation:

C_{1,t}=C_{1,0}*e^{-k}^{₁}^{t} (Eq. 35)

The accompanying release characteristics of MIP 2 can similarly be expressed as follows (Trace 2):

C_{2,t}=C_{2,0}*e^{-k}^{₂}^{t} (Eq. 36)

And then the sum of the two can be taken to express the overall, or net behavior of the combined MIP matrices (1 and 2) for this example MIP system's combined release profile, which is as follows:

C_{Total,t}=C_{1,t}+C_{2,t}=C_{1,0}*e^{-k}^{₁}^{t}+C_{2,0}*e^{-k}^{₂}^{t} (Eq. 37)

In a related embodiment, a single MIP polymer or MIP matrix having two distinct sets of binding sites with the same characteristic release rate constants of the first example embodiment could also be used, and in the absence of any diffusional effects or limitations, would operate to provide a substantially identical release profile as the first example embodiment.

FIG. 2A shows a graphical illustration of a system in which the material whose concentration is to be controlled (denoted as black dots) is present in the fluid media (denoted as the white shaded region on the right side of each frame, 203) and at an initial time (T=0, Frame a) no material has been absorbed by the MIP matrix (denoted as the gray shaded region on the left side of each frame, 200). The entire surface of the MIP matrix is in contact with the surrounding fluid media, the interface being denoted by the dotted line 201 in each frame. After a time interval of T1, denoted by frame b, some of the material has been absorbed by the MIP matrix, the process continuing at time T2 in frame c and finally reaching a relative steady-state equilibrium condition denoted in frame d at time Tf.

In this present example, the capacity of the MIP matrix is selected to accommodate a relative total concentration of 40 mM/g of the material, representing a saturation point beyond which the MIP matrix cannot no longer adsorb any additional net quantity of materials, although it remains in equilibrium with the surrounding liquid media with some unabsorbed materials present therein. The amount of total MIP material present is about 1 g, and the volume of the fluid media is 1 L.

Correspondingly, FIG. 2B shows a plot of the material concentration within the MIP matrix as a function of time, for the series of example novel MIPs with suboptimal binding sties, revealing that the MIP matrix is adsorbing the material from the liquid media under generally first order kinetics, the net concentration of captured materials increasing to the point, T=Tf, at which the system is nearly at a steady-state level and the relative distribution of material absorbed onto the MIP matrix and that of free material remaining in the liquid media are relatively constant with respect to trace 1, which represents a MIP with the largest average associative binding constant, k₁, of about 0.1/min. It is noted that the other MIP systems, with kₘ values that are progressively smaller, adsorb the free material from the media at a much slower pace, not quite achieving a steady state or equilibrium adsorption condition within the 360 min time frame contemplated here. Accordingly, it can be seen that employing MIPs that have at least significantly different average associative binding constants can enable the time-dependent release (or adsorption) of a material to adjusted. By employing a MIP with two or more different k values, careful selection of the k values will enable the MIP or MIP system to exhibit desired programmed time-delay (or adsorption) profiles by means of the MIPs or MIP sites with different k values acting in synergy to control the equilibrium distribution of a material between themselves and the fluid media in which the MIPs are in contact.

In another embodiment of the present disclosure, shown graphically in FIG. 3A, a MIP system (300) having two similar, but significantly different average associative binding constants (k₁ and k₂) as shown in FIG. 2, are employed, both shown by open circles (i.e. not visually distinguished), initially empty and in communication (301) with a fluid media (303) containing the material (black dots) whose concentration in the media is to desirably be control. In FIG. 3B, one embodiment employing a single MIP matrix that has been imprinted so as to exhibit two sets of binding sites having two significantly different average associative binding constants is explored. Here, trace 1 shows the relative amount of material over time, or the adsorption profile of one set of binding sites having an average k₁ or 8.0×10⁻²/min, while trace 2 shows the relative amount of material absorbed over time by the second set of binding sites having an average k₂ or 4.0×10⁻²/min, which is smaller and thus correspondingly absorbs material from the media and exchanges bound material at a slower rate than the first set of binding sites. The combined adsorption profile of the two MIPs acting in concert is shown by trace 3, which represents the actual adsorption profile over time of the novel MIP system having two unique and significantly different kₘ values.

In FIG. 3C, a closely related embodiment to that shown in FIG. 3B is explored, here the MIP system being composed of two separate MIPs or MIP matrices, each having its own corresponding kₘ value, which are identical to those in the system presented in FIG. 3B. It is to be noted that the resulting adsorption profiles of each of the separate MIPs denoted by trace 1 and trace 2 are identical to those seen in FIG. 3B in which a single MIP matrix had two sets of binding sites imprinted within it. Again, the overall adsorption profile, shown by trace 3, is the sum of the contribution of the component MIPs, being the same as the prior example. This illustrates the utility of the present disclosure in being able to combine separate MIPs or MIP matrices into MIP systems that operate to perform controlled time release and controlled time adsorption of a selected material, in addition to using a single MIP that has been imprinted to exhibit a plurality of distinct binding sites. A further advantage of combining separate MIPs or MIP matrices is that a wide combination can be contemplated, as well as the ability to adjust the relative proportion or weight of MIP materials present, controlling the binding or release capacity of the MIPs and the MIP system as well with respect to the selected material whose concentration in a fluid system is to be controlled.

By contrast, a conventional, MIP matrix formed using the unmodified TIE under optimal conditions would tend to exhibit an average associative binding constant having a magnitude significantly greater than that of the MIP systems of the present disclosure that depend on suboptimal binding sites and correspondingly lower average associative binding constants with respect to the material whose concentration in a fluid media is to be controlled. Thus, the conventional MIPs would tend to adsorb all free material extremely rapidly and not maintain an equilibrium or steady-state value of material in the media, and conversely, if dosed with the TIE material, tend not to release that material under practical timeframes. Further, any depletion of the material in the fluid media in contact with a traditional MIP matrix using an unmodified TIE would be substantially permanent, as the absorbed materials would not be released from the MIP matrix due to its high associative binding constant, preventing the conventional systems from being used effectively for maintaining a consistent, and non-zero material concentration in the fluid media in contact with the MIP system, even if selected to have similar limiting material binding capacities (Cₘₐₓ).

Accordingly, without changing the capacity of the MIP system, and by merely changing one of the MIP component's binding affinity (or incorporating binding sites within the MIP matrix of different affinity), the present novel systems can readily be tailored to provide a system that exhibits the desired uptake (or release) profile of any selected material, by using a MIP system exhibiting at least two or more unique, and significantly different average associative binding constants with respect to a selected material, wherein those two or more binding constants are suboptimal in value compared to the binding affinity of the MIP system with respect to the TIE material used in its formation.

Further, using the present novel approach of selecting the relative values of two significantly different MIP binding sites, one can readily tailor a system to provide for a desired steady state or equilibrium level of a material in a fluid media in contact with an novel MIP matrix.

It is important to note that these embodiments illustrate an important feature of the present novel approach in that the MIP systems employing two or more different binding sites (with significantly different associative binding constants than that exhibited by a MIP formed with an unmodified TIE) have utility in controlling the fluid media concentration of a selected material of interest, without relying on the ultimate capacity of the MIP system to limit material adsorption. Said another way, this enables an additional degree of freedom in designing and using MIP systems without the limiting value of the MIPs material binding capacity to be a controlling factor. However, the additional advantage of the present novel MIP systems is that the material binding capacity of the MIPs employed can also be used to modify the behavior, providing a more robust system with additional options for tailoring and controlling the rate of release and adsorption of any desired material into and out of a fluid media, as desired.

In another embodiment of the disclosure, a MIP system is designed to release a first material while catching or adsorbing a second, molecularly similar material present in the surrounding fluid environment. A particularly beneficial application would enable the dosing of a drug to a patient, for example, while adsorbing any unwanted, interfering or contra-indicated material that might be present in the patient's stomach, intestine or blood stream. For example, theophylline is a molecular compound often used in oral form for the treatment of breathing disorders, such as chronic obstructive pulmonary disease (COPD). However, caffeine is contra-indicated when taking theophylline, as it can increase the side effects of the drug, causing nausea, vomiting, insomnia, tremors, restlessness, uneven heartbeats, and seizure (convulsions).

In another embodiment, a MIP system illustrated as in FIG. 4A can be selected that has two significantly different k values with respect to caffeine adsorption, and simultaneously has two significantly different k values with respect to theophylline release. Here, MIPs having a first set of k values with respect to caffeine of 7.0×10⁻²/min and 5.0/min is selected in which the second set of k values with respect to theophylline is 1.0×10⁻²/min and 0.5/min. Initially, as illustrated in frame (a) of FIG. 4A, the theophylline (denoted by shaded circles) is loaded onto, or pre-absorbed, by the MIP matrix (400) approximately to the saturation point in this example, or about a level of 40 mM/g of theophylline in the MIP matrix, although optionally the degree of loading can be varied in order to change the overall behavior of the system as desired. Also shown in frame (a) is a fluid media (403) in contact with the MIPs matrix via the surface or interface (401) of the MIP matrix (400), the fluid containing undesired caffeine molecules (denoted by the shaded squares), at a concentration providing a total amount of about 40 mM of caffeine present. The remaining frames (b)-(d) show the system at various stages in time following the initial contact, illustrating the overall tendency of the MIP matrix to release the less tightly absorbed (lower k values) theophylline material and to adsorb the more tightly absorbed (higher k values) caffeine material over time as T progresses from T1 to T2 to a final approximate equilibrium state, Tf.

In FIG. 4B, the instantaneous concentrations of the two materials present within the MIP matrix are shown as a function of time, trace 1 corresponding to caffeine and trace 2 corresponding to theophylline. The vertical lines indicated as (a)-(d) correspond in time to the respective frames (a)-(d) as illustrated in FIG. 4A. Here, it is seen that initially, the theophylline concentration begins to decrease in the MIP matrix as material is released into the surrounding fluid media. In contrast, the MIP matrix shows a slight lag in adsorbing any caffeine material from the fluid media, due to the fact that in this embodiment, there were few, if any, additional caffeine binding sites that were not previously saturated with theophylline. Thus, there is a slight delay in caffeine adsorption because the sites must first desorb some theophylline to open up or make available, binding sites for the more highly associative (higher k) caffeine molecules. However, after this initial delay, the adsorption behavior of caffeine into the MIP matrix essentially mirrors the simultaneous desorption of theophylline from the MIP matrix into the surrounding matrix. At a point in time closely following T=T₁, illustrated by frame (b) of FIG. 4A and the dotted line (b) in the present figure, the level of caffeine absorbed begins to exceed that of the theophylline remaining in the fluid media. Finally, after some time, Tf, the system is nearing an approximate equilibrium state wherein nearly all of the theophylline has been released from the MIP matrix, which has then absorbed nearly all of the free caffeine present in the surrounding fluid media. Accordingly, this example embodiment illustrates one approach to delivering a material to a fluid environment while simultaneously removing (adsorbing) a second material.

In FIG. 5A, another embodiment of the present disclosure is explored. Here, the MIP matrix and fluid media conditions are identical in nearly all respects to the prior embodiment illustrated in FIGS. 4A and B. However, in this present embodiment, the MIP matrix is evenly divided into two components (right and left 502), of equal weight, being 0.5 g each. One of the component halves of the MIP matrix is coated with a delayed release material (508) that is slightly soluble in the fluid media (503), such that the delay release material coating will remain substantially intact for about 1 hour (60 minutes) and then be effectively breached by partial dissolution sufficient to enable the surrounding fluid media to contact at least a portion of the second coated MIP matrix component. The first component half (right 502) is uncoated and remains in full contact via its uncoated surface or interface (504) with the fluid media (503) throughout the time period. In contrast to the previous embodiment illustrated in FIGS. 4A and B, the FIG. 5 system behaves markedly different in that essentially all the free caffeine (denoted by shaded squares) initially present in the fluid media is absorbed by the uncoated (504) first MIP matrix component (right 502) within a fairly short time, as shown in trace 1 which represents the instantaneous concentration of caffeine in the fluid matrix as a function of time. Indeed, after about 30 min. there is substantially no caffeine remaining in the fluid media, having been absorbed by the uncoated first MIP matrix component. Note that in this example, there was no initial delay as seen in the prior embodiment wherein theophylline molecules had to first desorb from the MIP matrix to leave behind unoccupied binding sites for eventual caffeine adsorption to then proceed. Further, the rate of caffeine adsorption is no longer dependent on the concomitant release of theophylline from the MIP matrix, and thus proceeds fairly rapidly resulting in the near total adsorption of all caffeine initially present in the fluid media.

After a delay of about 60 min (denoted by vertical line c), the delay release coating (508) surrounding the second MIP matrix component (right 502) is breached by the fluid media exposing this second MIP material that has been pre-loaded with theophylline (denoted by shaded circles), which then begin to be desorbed into the fluid media (503), as shown by trace 2. Surprisingly, without the interference of competing caffeine molecules, even though the latter might have been expected to accelerate desorption owing to the higher association constant of the MIP matrix binding sites for caffeine (thus essentially displacing the less tightly bound theophylline molecules), it is seen instead that the theophylline is released much more rapidly. Accordingly, by about 90 min, well before point (d) is reached, essentially all the theophylline has been released from the first MIP component half (right, 502) into the surrounding fluid media. Thus, this example embodiment shows that an additional novel feature may optionally be included, being the use of a barrier coating on one or more of the novel MIP matrices that enables a time-delay or control-release, or inversely, a timed adsorption or controlled adsorption event to further utilized in order to produce a desired adsorption/desorption profile of one or a plurality of different materials associated with the novel MIP matrices.

In this present embodiment, the use of a time-delay coating on one of the MIP matrix components would enable a medicine such as theophylline to be released into a patient's stomach/intestinal track only after the levels of any competing, contra-indicated caffeine present was reduced to zero or some minimum desired level.

Following are two example embodiments of the present disclosure, utilizing the novel MIP systems with a delay or control-release coating on one or more MIP components in order to provide a delayed release of a medicine while simultaneously adsorbing a second molecular from the fluid media into which the medicine is desired to be released.

In FIG. 5C, an example embodiment of the novel MIPs present in a tablet style dosage form 510 for oral delivery of theophylline to a human patient is presented in diagrammatic fashion, the view corresponding to a cross-sectional view taken midpoint through said tablet. Here, the tablet style dosage form 510 has a first MIP component 512 that has at least one associative binding constant for caffeine that is sufficiently large in value so that the first MIP component 512 is able to adsorb its total binding capacity of caffeine when exposed to a fluid media having free caffeine molecules present in the fluidic solution within the desired time frame for medicine delivery. The first MIP component 512 can optionally be coated with a protective film or binding aid in the form of a first coating 514 that in this example dissolves quickly in the fluid media without offering any time-delay properties. A second MIP component 516 present features a least one associate binding constant for theophylline that is sufficiently small in value so that the second MIP component 516, when it is exposed to the fluid media, is capable of releasing substantially all of the previously dosed (absorbed) theophylline present within that MIP component. The second MIP component can optionally be coated with a time-delay or control-release coating, and in this example is coated with a time-delay second coating 518 that remains intact after the tablet style dosage form 510 disintegrates until such time as it is breached by the fluid media to expose the second MIP component 516 material to the fluid, the choice of coating, application method and thickness being selected so that the average time to breach is within a desired time period following ingestion or introduction of the tablet to a patient. In this present embodiment, the two MIP components 512 and 516 are in the shape of a short circular cylinder or half-tablet style dosage form and are immediately adjacent and aligned with respect to one another, optionally bound together with a suitable binding material present at their interface in order for the resulting tablet style dosage form 510 to maintain structural integrity. Optionally, the tablet can be coated with an outer coating 520, if desired to provide additional features to the example embodiment, such as a binder coating for structural integrity, an enteric coating to prevent dissolution within the stomach, a delay-release coating to ensure delayed dissolution for a selected time period, etc. Naturally, in other related embodiments, the structure, orientation, shape, size and coating options for the first and second MIP components 512 and 516, respectively, can be varied as desired for the particular application needed.

For example, in another related embodiment, a capsule style dosage form is presented in which the novel MIP materials are present in the form of small beads, optionally coated, which are in turn packaged within a tertiary outer container or capsule, such as a two section gelatin capsule familiar to the art.

FIG. 5D shows a diagram corresponding to a cross-sectional view of a capsule style dosage form 521 holding a plurality of beads (not shown to scale). The beads present include a plurality of beads composed of a first MIP component 523 and a second MIP component 527, both in the form of essentially rounded spheres, contained within a lozenge shaped and thin-walled, two part outer capsule comprising a male section 531 and a female section 543 into which the male section 531 frictionally slides and engages in a closed position, retaining the beads within its confines. The beads, coating thicknesses and capsule wall thicknesses are not drawn to scale. In this present embodiment,

Here, the capsule style dosage form 521 has a first conventional MIP component 523 that has at least one associative binding constant for caffeine that is sufficiently large in value so that the first MIP component 523 is able to adsorb its total binding capacity of caffeine when exposed to a fluid media having free caffeine molecules present in the fluidic solution within the desired time frame for medicine delivery. The first MIP component 523 is in the form of a plurality of spherical beads which can optionally be coated with a protective film or binding aid in the form of a first coating 525 that in this example dissolves quickly in the fluid media without offering any time-delay properties. A second, novel MIP component 527, present also in the form of a plurality of spherical beads, features a least one associate binding constant for theophylline that is sufficiently small in value so that the plurality of second MIP components 527, when it is exposed to the fluid media, is capable of releasing substantially all of the previously dosed (absorbed) theophylline present within that MIP component. Accordingly, following ingestion, once the outer capsule sections 531 and 533 dissolve or disintegrate sufficiently so as to be breached, the plurality of first MIP component 523 beads and second MIP component 527 beads are released from confinement to interact with the surrounding fluid media.

The beads comprising the second novel MIP component 527 can optionally be coated with a time-delay or control-release coating, and in this example embodiment are coated with a time-delay second coating 529 that remains intact after the capsule style dosage form 521 disintegrates, which occurs when the outer capsule sections 531 and 533 dissolve or disintegrate sufficiently so as to release the payload of MIP beads. The second coating 529 is selected as before to dissolve or be substantially breached at some selected average time following exposure to the media, at which point the theophylline laden second MIP component 527 begins to release the medicine to the surrounding fluid media, such as in the stomach or intestines of the patient receiving this dosage form, for example.

Of course, in other related embodiments, the structure, orientation, shape, size and coating options for the first and second MIP components 523 and 527, respectively, can be varied as desired for the particular application needed. This present embodiment illustrates that the novel MIP materials can be used in conjunction with a time-delay or control-release coating in order to control the update and release of target materials from and into a fluid media, respectfully.

In FIG. 6A, another embodiment of the present disclosure is explored in which a MIP matrix (602) has been imprinted with two different binding sites, represented by open (white) squares and circles on the left side of each frame. Two distinct materials, represented by black circles and black triangles are present, the first material (black triangles) having been preloaded onto the MIP matrix, while the second material (black circles) is initially present in the liquid media (603) in contact with the MIP matrix, whose surface or interface is represented by the dotted line 601. After some time has passed (frame b), the system has reached an approximate equilibrium and nearly all the first material has been released by the MIP matrix into the media, while most, if not all, of the second material present in the media has been absorbed by the MIP matrix.

In FIG. 6B, the relative concentrations of the first and second material within the MIP matrix are shown as a function of time over 360 min. Here, a first MIP site (white squares) has associative binding constants of 7.0×10⁻²/min and 1.0/min with respect to the first material (black triangles) and the second material (black circles), respectively, while a second MIP site (white circles) has associative binding constants of 5.0×10⁻⁴/min and 0.1/min with respect to the second material (black circles). It is seen that initially, the MIP sites preloaded with the first material to its saturation point of 20 mM/g begins to desorb or release that material into the surrounding fluid media owing to the low binding affinity, and within a short time period of less than about 36 min., nearly all the first material has been released, as shown by trace 2 in FIG. 6B. Simultaneously, the higher affinity MIP binding sites (with respect to the second material) results in a fairly rapid adsorption or catching of the second material from the fluid media into the MIP matrix, and after about 100 min., nearly all the second material has been absorbed from the media, up to its saturation point of 40 mM/g with respect to that second material.

In FIG. 6C, the relative concentrations of the first and second material within each of the two separated MIP matrices (604 and 606 in FIG. 6A frames c and d) are shown as a function of time over 360 min., both MIP matrices having unique MIP sites corresponding to the first and second materials, and also having the same associative binding constants as the embodiment presented in FIG. 6A frames (a) and (b) and in FIG. 6B. The only difference is that the two separate MIP matrix materials (604 and 606) have only a single molecular-type imprint rather than the single MIP matrix of example FIG. 6B having both types of binding sites within the same MIP matrix. Here, trace 1 and trace 2 show that the respective catching and release (adsorption and desorption) behavior of this embodiment is essentially identical to that exhibited by the mixed site MIP matrix embodiment. This illustrates that, providing that the MIP matrix materials are in contact via their surfaces or interfaces (collectively 601) with the fluid media (603), that adsorption and desorption kinetics unique to the MIP binding sites govern the equilibrium catch and release behaviors of the novel MIP matrices, providing even greater flexibility in that a plurality of separate MIP matrices, each having a unique imprinted binding site and corresponding associative binding constants, may be combined merely by physical combinations of separate physical polymer matrices in order to practice the present disclosure.

In a further example of the disclosure, another embodiment graphically illustrated in FIG. 7A features two MIP matrices (704 and 706) representing a MIP system having two different sets of binding sites, a first set of sites shown by empty white squares and a second set of sites represented by empty white circles, that are both preloaded with a material (illustrated as solid black triangles) to be released into the fluid media (703) in which the MIP system is submerged and its entire surface 701 interface in contact with the media. The vertical slashed line 703 in FIG. 7A is meant to illustrate that the two MIP matrix materials can be combined in any suitable fashion, including being a MIP matrix having both sets of different binding sites present, or separate MIP components having one only set of binding site each combined physically, such as for example, but not limited to mixed powders, mixed fibers, layered structures, coated substrates, webs, foams, and the like. Here, the two MIP matrices have binding sites exhibiting unique associative binding constants of k1=2.0×10⁻⁴/min and k2=0.5/min; and k3=5.0×10⁻⁴/min and k4=0.1/min, respectively, representing a stronger binding MIP matrix (706) and a weaker binding MIP matrix (704) with respect to the target material to be released (illustrated as solid black triangles), as shown in FIG. 7B.

In FIG. 7B, trace 1 shows the relative amount of preloaded material that is released into the fluid media, being the instantaneous fractional concentration released by the MIP matrix 706 with the relatively higher associative binding constants, while trace 2 shows the instantaneous fractional concentration of material released by the less associative MIP matrix 704, which has the smaller average associative binding constants with respect to the material being released. Comparison of traces 1 and 2 reveal the behavior of the two different MIP matrices with respect o the dosed material, the second MIP matrix 704 acting to nearly completely release its payload of material within 36 min of contact with the fluid media, indicated by time point (c), corresponding to frame 3 in FIG. 7A. In contrast, the first MIP matrix 706 has a greater overall affinity for the material, and tends to release it slower than does the MIP matrix 704. Accordingly, trace 3 shows the total concentration of the material in the fluid media over time, and thus reflects the overall release profile of the novel MIP system ensemble, a release profile that is unique to the novel system, and which is fully adjustable in regards to the desired speed and extent of material delivery, by selecting the associative binding rate properties of the two MIP matrix components, their relative proportions, and their relative loading capacities.

In a further embodiment of the present disclosure, a MIP system is explored that delivers a delayed step function release profile of a desired material into a fluid media, as shown in FIG. 8. In FIG. 8, the release of theophylline into a fluid media, such as for example stomach and intestinal fluids, is shown for an novel MIP system that has been designed by iterative modeling calculations to identify the required sets of average associative binding rate constants and relative molar proportions of a plurality of MIP matrices which when combined will deliver the ‘theoretical’ or desired release profile as shown in trace 803 by the solid line. The desired release profile 803 features a desired initial constant (steady state or zeroeth order) release target dosage rate 801 of 0.01 mM/min for an initial time period of from time zero (T=0) to about 60 min., followed by a stepped-up constant desired release target dosage rate 802 of 0.05 mM/min after about 60 min. and continuing until the MIP system is depleted of releasable material. Iterative calculations according to the present disclosure, converged after about 50 iteration steps (note that multiple iterations within these steps occur as part of the built in optimization routine) to a best fit dosage-response profile 804 shown by the connected dotted line in FIG. 8.

The best fit value of the model results compared to the desired release profile was determined to have a root-mean-square (RMS) error value of less than 0.000198 mM/min, showing at degree of dosage control precision of about +/−2.0% with respect to the low dosage target range (100%×0.000198/0.010) of 0.01 mM/min, and of about +/−0.4% with respect to the delayed high dosage target ranged (100%×0.000198/0.050) of 0.05 mM/min. Further iteration steps using the novel MIP calculations typically result in only slightly reduced RMS error and calculated average associative binding rate constants and relative molar proportions that are within the tolerance range of experimental error, so that there is no need for continued iterative refinement to determine target values of these parameters to be used to design a MIP system with the desired release profile.

In this embodiment, the MIP system consists of a plurality of five MIP matrices, whose selected associative binding rates (kₓₘ) (see column 1 of Table 1) were calculated starting with initial seed values as shown in column 3 of Table 1. In addition, initial seed values for the physical parameter constraints were selected, including the molar proportion of each MIP matrix or unique collective MIP binding site, and a delay parameter associated with each MIP matrix relating to the average delayed release time of a degradable protective coating or release layer on that respective MIP matrix. The molar binding capacities of the MIP matrices were held at fixed values for the calculation, being a constraint on the system, and enabling the molar proportion of each MIP matrix in the system to be calculated without codependency on this factor. Accordingly, Table 1 shows the initial and optimized kₓₘ values for a MIP system of 1 gram total polymer weight, to deliver an active theophylline material (m) to an aqueous fluid media, with some initial estimated kₓₘ values (see Table 1 note 2) and constraint ranges (note 1) imposed on the resulting calculated optimized kₓₘ values (note 3) of a MIP system capable of releasing the theophylline payload in a manner matching the desired release profile 803 shown in FIG. 8. It is to be noted that the “choppiness” in the calculated release profile trace 804 is partly owing to the iterative calculation approach employed and the incremental time unit of approximately six (6) min intervals used. Selecting additional iterations and/or selecting a smaller incremental time unit, say between 0.1 min to about 1 min would result in the calculated profile being smoother and converging faster to match the initial target release profile, only requiring a greater number of iterative calculations. However, the choice of the incremental time unit is dependent on the overall time period of catch or release desired, and larger increments are preferred for greater time periods to prevent unnecessary calculation where little additional improvement in optimization is achieved. For fairly short overall time periods of catch or release, a correspondingly smaller increment is preferentially used in order to converge to the optimized solution that better matches the desired response profile.

TABLE 1
Optimized Associative Binding Constants of MIP System with Five
MIP Components (Xₘ)

| MIP | | | |
|---|---|---|---|
| Component | | | |
| kₓₘ | Constraint (1) | Initial Kₓₘ (2) | Optimized Kₓₘ (3) |
| (Rate Constant) | 0 < k_{Xm} < Y | (mM/min⁻¹) | (mM/min⁻¹) |
| k_{R1} | 0 to 1.00 | 1 × 10⁻⁵ | 0.00221 |
| k_{R2} | 0 to 1.00 | 3 × 10⁻⁶ | 0.00244 |
| k_{R3} | 0 to 1.00 | 1 × 10⁻⁶ | 0.00289 |
| k_{R4} | 0 to 1.00 | 2 × 10⁻⁴ | 0.00323 |
| k_{R5} | 0 to 1.00 | 8 × 10⁻⁴ | 0.00265 |
| k_{C1} | 0 to 10.00 | 1 × 10⁻⁶ | 0.07183 |
| k_{C2} | 0 to 10.00 | 4 × 10⁻² | 5.315 |
| k_{C3} | 0 to 10.00 | 4 × 10⁻³ | 0.20945 |
| k_{C4} | 0 to 10.00 | 7.5 × 10⁻⁴ | 4.584 |
| k_{C5} | 0 to 10.00 | 6 × 10⁻³ | 0.0865 |

(1) Imposed constraint value of 0-1.0 for lower associative binding range for “releasing” MIP sites, and 0-10.0 for higher associative binding range for “catching” MIP sites.
(2) Initial values from database of collective MIP matrix associative binding constants derived from actual, experimental or modeled kinetic parameters for a particular polymer, porogen and TIE patterned MIP matrix.
(3) Calculated values representing optimized average associative binding constants for each MIP matrix constituting the MIP system.

In Table 2, the optimized mass fractions (see column 3, note 2) of the MIP system component MIP matrices corresponding to a set of “release” MIPs and a set of “catch” MIPS (see column 1) are shown along with the initial constraints (column 2, note 1) imposed on the system. Here, the initial seed values for each of the Mₓₙ values was an equimolar 0.2 unit value, so that the five (5) MIP matrices comprising the MIP system add up to a total mass fraction of 1.0, being unitless and a further constraint on the system, as this value represents the relative proportion of each MIP matrix with its own characteristic kₓₘ values as needed for the collective MIP system to deliver the desired release profile of theophylline in this novel embodiment. In this particular novel embodiment, each catch and release set of MIP matrices is also initially constrained to have equal weights in the system, although this constraint could also be modified by allowing the relative proportions to vary as well in other embodiment. In this present embodiment, having this catch and release ratio fixed (1:1 or equal weight) enables any resulting calculated k values to be combined if within experimental error, for a simpler solution to the target dosage profile. For example, if two optimized k values for a MP matrix are not significantly different or are not different within measureable experimental error, then the model and resulting system can be simplified by substituting the additive quantity resulting from combining the mass fractions of the two particular MIP materials with essentially similar k values. In this particular embodiment shown in FIG. 8 and Table 1, the individual MIP matrix component kₓₘ values all differ significantly by at least 1×10⁻² and accordingly, cannot be combined to simplify the resulting system.

TABLE 2
Optimized Mass fractions of MIP System

| MIP Component | | | |
|---|---|---|---|
| M_{Xm} | Constraint (1) | Optimized M_{Xm} (2) | |
| (Mass fraction) | 0 < M_{Xm} < 1.0 | (unitless) | Total (3) |
| R1 | 0 to 1.00 | 0.354 | |
| R2 | 0 to 1.00 | 0.206 | |
| R3 | 0 to 1.00 | 0.150 | |
| R4 | 0 to 1.00 | 0.114 | |
| R5 | 0 to 1.00 | 0.186 | |
| TOTAL R1-R5 | 1.00 | | 1.010 |
| C1 | 0 to 1.00 | 0.562 | |
| C2 | 0 to 1.00 | 0.110 | |
| C3 | 0 to 1.00 | 0.080 | |
| C4 | 0 to 1.00 | 0.124 | |
| C5 | 0 to 1.00 | 0.128 | |
| TOTAL C1-C5 | 1.00 | | 1.004 |

(1) Imposed constraint value of 0-1.0 for each individual mass fraction of that MIP matrix component, with the additional constraint that the total additive molar fraction of the collective sums to a value of 1.
(2) Initial values were arbitrarily set at 0.2 for each.
(3) Calculated optimized values are summed, with a target theoretical value of 1.0. Each catch and release set of MIP matrices is also given equal weight, being present in equal molar quantities.

In Table 3, the optimized coating delay factors for the example novel MIP system of FIG. 8 is shown are shown in column 3, wherein column 1 represents the delay factor for each particular MIP component D_{Xm} (min), while the constraint range value imposed on the iterative calculation is shown in column 2. The optimized set of delay factors, Dₓₘ, for the respective MIPₓₘ matrix components in some instances, converge to a zero value, such as for example R4 and C4 as shown in Table 3.

Accordingly, these particular MIP matrix components do not require a delay-release coating in the final MIP system. Further, some delay factors converge to the same or very close optimized value, indicating that the corresponding MIP matrices could be combined into a single system and coated with the same delay-release coating, optionally to simplify processing and reduce the number of coating steps required in formulation a controlled release MIP system. For example, in another embodiment, the three MIP matrices or components corresponding to R4 and C4 as explored above, could further be combined with C3, as its delay release factor of 2 min may be within the range of experimental error or close enough that the overall release profile would be essentially equivalent to the desired profile.

In yet another example embodiment, MIP components R1 and R2 could be combined and coated with a delay-release coating providing a 10 min delayed onset release mechanism, while MIP components R5 and C1 could similarly be combined and coated with a delay-release coating providing a 24 mm delayed onset release mechanism.

Alternatively, in another embodiment, the three MIP matrices or components could be physically combined because MIP component C2's optimized value is very close to that of R5 and C1, and the combined MIP matrices physically comingled and then coated with a single delay-release coating providing a 24 or 25 min delayed onset release mechanism could be employed without significantly altering the desired release profile.

Alternatively, in yet another novel embodiment, a single MIP matrix exhibiting the three respective binding sites with their representative kₓₘ values having the requisite number of sites present in a ratio corresponding to the ratio of their optimized molar ratios could be produced as a single MIP polymer matrix, which in turn could then be coated with a single delay-release coating providing a 24 or 25 mm delayed onset release mechanism could be employed without significantly altering the desired release profile.

In all these novel embodiments, the calculations could be repeated with the combinations described above chosen as model constraints, in order to fine tune the system or to seek alternative embodiments with fewer separate components required, and/or fewer separate coatings required in order to accurately reproduce and deliver the desired release profile initially sought.

TABLE 3
Optimized Coating Delay Factors for MIP System

| Delay Factor For | Constraint (2) | Optimized |
|---|---|---|
| MIP Component (1) | 0 < k_{Xm} < Z | D_{Xm} (3) |
| D_{Xm} (min) | (min) | (min) |
| R1 | 0 to 60 | 10 |
| R2 | 0 to 60 | 10 |
| R3 | 0 to 60 | 41 |
| R4 | 0 to 60 | 0 |
| R5 | 0 to 60 | 24 |
| C1 | 0 to 60 | 24 |
| C2 | 0 to 60 | 25 |
| C3 | 0 to 60 | 2 |
| C4 | 0 to 60 | 0 |
| C5 | 0 to 60 | 8 |

(1) Delay factor for MIP component of matrix or system
(2) Constraint on delay factor for a targeted change in release profile after 60 minutes
(3) Optimized delay factors for individual MIP component indicating the average time to release of a coating.

In another novel embodiment, a MIP system is presented that exhibits a selected initial high dosage steady-state release profile for a first period of time followed by a subsequent delayed step-down to a second, lower dosage release profile for a second period of time with respect to the controlled release of a material (theophylline in this example) into an aqueous fluid media, as shown in FIG. 9. Here, the desired release profile 903 features an initial high steady-state or constant release (901) of material at a rate of 0.08 mM for a period of 60 minutes, followed by a delayed lower dosage, but also steady-state or constant release (902) of the same material at a rate of 0.01 mM/min for a period of at least an additional 300 minutes or until the amount of available material within the MIP system is depleted or released into the aqueous fluid media.

Again, using a plural MIP matrix model, with five components contributing as “release” MIPs (R1 through R5) and five components contributing as “catch” MIPs (C1 through C5), with delay functionality included, the model calculations were applied and after fifty (50) iterations of calculations, the model converged to the optimized values shown in Table 4 for the set of average associative binding constants, Kₓₘ, mass fractions, M_{Xm}, and corresponding delay factors, D_{Xm}, providing a good fit with respect to the desired release profile 903 discussed above. Again, it is noted that there is some choppiness in the calculated release profile, notably in the initial release period 901 as seen in FIG. 9. Additional iterations serve to reduce the apparent fluctuations, but as discussed hereinabove, the optimized calculated values do not change significantly with additional iterations, confirming that the likely actual release profile will behave like the average value of the calculated release profile shown (see dotted line trace 905 reflecting the average calculated release profile), and that the apparent variations are due to the iterative nature of the calculations and the chosen time interval of 10 mm incremental time units.

TABLE 4
Optimized MIP System Parameters for High/Low Step-Down Steady
State Dosage Profile

| | | Optimized | |
|---|---|---|---|
| MIP Component | Optimized kₓₘ (1) | M_{Xm} (2) | Optimized D_{Xm} (3) |
| Xm | (mM/min⁻¹) | (unitless) | (min) |
| R1 | 0.01466 | 0.5491 | 49 |
| R2 | 0.02592 | 0.0556 | 6 |
| R3 | 0.01155 | 0.1890 | 0 |
| R4 | 0.00114 | 0.1664 | 31 |
| R5 | 0.02393 | 0.0307 | 3 |
| C1 | 0.14252 | 0.5960 | 10 |
| C2 | 0.69756 | 0.0628 | 14 |
| C3 | 0.12400 | 0.1161 | 12 |
| C4 | 0.30777 | 0.1762 | 11 |
| C5 | 5.23150 | 0.0589 | 12 |

(1) For a MIP system with total 40 mM capacity for theophylline, with calculated total gram weight of G_{mC} = 0.0220 gm, with an RMS Error = 0.000302.
(2) Summation of mass fraction composition of MIP system shows total F_{R} = 0.9910 and total F_{C} = 1.010.
(3) Note several very close delay factors for multiple separate MIP components.

In another embodiment of the present disclosure, a ramp-up release profile is explored in which the MIP system is tailored to produce a linearly increasing (“ramp up”) dosage release rate over a period of time, rather than a zero order or steady-state release profile as described hereinabove. In FIG. 10, this release profile is illustrated as trace 1003, which begins with an initial steady-state (zero order) release target 1001 of 0.04 mM/min of theophylline into an aqueous media for a period of about 120 minutes, followed by a drop in release to a value of about 0.01 mM/min initiating a ramped or linearly increasing dosage profile 1002 releasing material at an accelerating release rate of about 1.25×10⁻⁴ mM/min², corresponding to a ramp from 0.01 mM/min to 0.04 mM/min (0.03 mM change) over a 240 min time period). After 240 minutes, the amount of preloaded dosant (here, theophylline) would become essentially depleted from the MIP system, and the programmed release rate would drop to zero and terminate. Naturally, changing the MIP system parameters would enable changing the release characteristics to either shorten or prolong the material delivery window as desired, modify the time at which the ramp-up dosage regime begins, as well as the rates of release over the desired time window. For the model shown in FIG. 10, Table 5 shows the optimized values for a MIP system with five MIP catch components and five MIP release components resulting from the novel iterative calculations described herein.

Table 5 shows the calculated average associative binding constants, kₓₘ, mass fractions, M_{Xm}, and corresponding delay factors, D_{Xm}, for a MIP system whose release profile provides a very good match with respect to the desired release profile 1003 discussed above. Here again, several of the MIP matrix components require no delay functionality, enabling components R2 and C5, for example, to be used without a delay coating. Further, several MIP matrix components have very close delay factors, which would provide an option to combine the component MIP matrices within a partial MIP system and coat that system with a shared and common delay release coating, for example MIP matrices C1, C2 and C4 could optionally be combined and coating so as to have a delayed contact with the fluid media of between about 20-23 minutes after contact, without substantially altering the delivered release profile.

In further embodiments of the novel approach described here in constructing MIP systems with a desired catch and release characteristics capable of accurately achieving any desired dosing profile (including controlled and delayed adsorption and/or desorption of a material), one may optionally model simpler systems in which the number of MIP matrix components is reduced. Earlier example embodiments presented featured a dual MIP matrix component having a single set of “catch” and “release” type of kinetics, as well as more complicated systems in which a plurality of MIP matrix components are required in order to achieve more sophisticated dosage profiles. In addition, in yet other embodiments of the present disclosure, MIP systems employing a plurality of MIP matrix components with coatings or some other means of delaying the contact time of a particular MIP matrix component with another component or with the fluid media, may be employed. The coatings as well as other means of delaying the contact time as discussed above can be selected as desired from known art. Suitable means of delaying the contact time of a protected entity and an environment to which that entity is introduced that can be employed in this present disclosure can include any such means known in the art, including but not limited to films, coatings, layers, laminates, membranes, dissolvable capsules, containers, packaging, and the like, that either are activated, breached, compromised, dissolved, disabled, removed, or the like, in a time frame consistent with the required delay time for the particular novel MIP component or MIP system in which the delay feature is paired.

TABLE 5
Optimized MIP System Parameters for High/Low
Step-Down Ramp-Up Dosage Profile

| | | | Optimized |
|---|---|---|---|
| MIP Matrix | Optimized Kₓₘ (3) | Optimized M_{Xm} (2) | D_{Xm} (3) |
| Xm | (mM/min⁻¹) | (unitless) | (min) |
| R1 | 0.00134 | 0.19863 | 36 |
| R2 | 0.00602 | 0.23497 | 0 |
| R3 | 0.00247 | 0.19040 | 51 |
| R4 | 0.00126 | 0.20333 | 12 |
| R5 | 0.00196 | 0.18266 | 44 |
| C1 | 0.05334 | 0.56736 | 20 |
| C2 | 0.06039 | 0.14373 | 23 |
| C3 | 4.50550 | 0.07296 | 60 |
| C4 | 0.09875 | 0.10526 | 21 |
| C5 | 7.44861 | 0.10991 | 0 |

(1) For a MIP system with total 40 mM capacity for theophylline, with calculated total gram weight of G_{mC} = 0.0230 gm, with an RMS Error = 0.000178.
(2) Summation of mass fraction composition of MIP system shows total F_{R} = 1.0100 and total F_{C} = 0.9992.
(3) Note several very close delay factors for multiple separate MIP components.

Accordingly, these example embodiments are presented to show the wide range of both adsorption based and release based dosage control by the use of the novel MIP matrices and MIP systems in a fluid media to control and/or provide a programmable catch or release profile of a material into or out of, or the establishment of a desired equilibrium state, between a selected material with some degree of association with the MIP system and the fluid media in which the MIP system is introduced.

FIG. 11 shows one embodiment of an novel MIP modeling process in diagrammatic form detailing a process 1100 for determining optimized parameter values for an novel MIP system starting with a first step 1102 to select target catch or release profile as “seed” values for the initial MIP matrix parameters and system parameters that are initially looked up in a parameter table 1114 that is derived from a database of measured or experimental parameter values 1116, followed by successive iterative calculation steps 1106 through 1110 solving for a match to within a specified R target value (comparison step 1108) between the desired 1102 and calculated adsorption and/or release profile parameter values (1110) for one or more target materials, iterative calculations continued until a final optimized set of parameter values 1112 are derived within a desired R-square fitting tolerance, determined at step 1108, with respect to the desired profile.

In one embodiment of an novel process 1100 to determine the optimized set of MIP system parameter values 1112, if the R value is exceedingly poor with respect to the desired value(s), this suggests that the iterative calculations are non-converging or have converged on a localized, non-optimal minimum that requires the desired target profile to be modified in step 1118, either by changing the seed values, changing the number of iterations, changing the convergence conditions, and the like, and combinations thereof, in order to enable the calculations to iterate successfully to a global minimum solution with an good fitting R value to provide final optimized values 1112. Accordingly, one or more of a plurality of MIPs and MIP matrices and/or one or more MIP matrices with one or more of a plurality of optimized associative binding constants are then combined to produce the novel MIP matrix or MIP system that exhibits the desired programmed and time-delay profile for the particular material(s) selected. Once the MIPs and MIP matrix are synthesized and/or assembled, the actual measured (experimental) system parameters 1116 can be determined in step 1101 and stored in a searchable accessible database located on a computer drive, network drive or other similar data storage medium (1120) associated therewith, and these values used to update the parameter table 1114, to improve the accuracy and predictability of the novel MIP modeling process 1100.

In a series of figures, FIGS. 12A through 12F show the result of modeling an novel MIP system in order to achieve a desired controlled, time-delay dosage profile for theophylline with a delayed-step up release dosage capability, where an initial target release rate 1200 of 0.01 mM/min for about 60 mins is followed by a step-up to a higher target release rate 1201 of about 0.05 mM/min for an overall duration of about 360 mins before the dosed material is exhausted from the time-delay MIP system that is to be constructed using MIPs having the calculated values according to the novel MIP modeling process 1100 described hereinabove.

In FIG. 12A, modeling results showing the resulting dosage profile 1202 (denoted by connected dots as indicated) of a MIP matrix having two (2) significantly different kₘ values (average associative binding constants) with respect to theophylline is shown against the desired initial dosage rate 1200 and delayed release second dosage rate 1202. It can be seen that an novel MIP matrix exhibiting two kₘ values also does not provide the desired time-delay profile, although the general shape of the release profile is at least representative of the desired step-change.

Further, as seen in FIG. 12B, the use of four (4) kₘ values also does not provide the desired time-delay profile, although the general shape of the calculated release profile 1204 is at least representative of the desired step-change and the second release rate value is closer to the desired level. However, the use of six (6) kₘ values as seen in FIG. 12C does provide a calculated release profile 1206 that is very close to the desired profile, operating to deliver an initial dosage rate very close to the desired initial rate 1201, and a second time-delayed release rate very close to the desired secondary rate 1202.

Accordingly, modeling the novel MIP systems with a greater number of individual kₘ values results in successively better fits between the desired release rates and the actual release profile. As seen in FIG. 12D, the resulting release profile 1208 achieved using a MIP system with eight (8) kₘ values is very close to the desired target initial and secondary release rates 1201 and 1202. It is to be noted that the calculated values in the FIG. 12 series of novel example embodiments show some iterative fluctuations in calculated values owing to the incremental iterative value of approximately six (6) minute intervals selected for use in the novel optimization routine 1100. Selection of a shorter incremental iterative value of, say, one minute, would result in a smoother calculated profile, but would add additional iteration steps to novel optimization routine 1100. However, depending on the complexity of the desired release (or corresponding adsorption profiles for an novel “catching” MIP system), a shorter or longer incremental iterative value could be selected. By way of example, the selection of six (6) minute intervals here provided a total of 120 points (360 minute release profile over 120 steps of six minute intervals). In practice, any reasonable number of iterative steps and number of total iterations can be selected in order to calculate a desired release or adsorption profile for a given situation. Further, the results in FIG. 12 are presented as “connect the dot” data points, while a truer reflection of the calculated or anticipated release profile would be an average trend value or best fit equation between the collective set of individual calculated values (dots) shown for the calculated delivery profile 1208.

In one further embodiment, an novel MIP system employing ten (10) kₘ values is presented in FIG. 12E, showing a nearly perfect calculated release profile 1210 that very nearly duplicates the desired release profile, both in terms of the desired initial 1201 release rate and the secondary delayed release rate 1202.

FIG. 12F shows a bar chart of the overall root-mean-square (RMS) errors of the corresponding modeled MIP systems described above and presented in FIGS. 12A-E. An acceptable RMS error corresponding to +/−0.0005 as shown by the dotted line 1203 reveals that for the particular desired release profile exampled in the novel embodiment for FIG. 12, that a MIP system with at least six (6) or more individual kₘ values would provide an acceptable optimized overall release profile. As expected, the greater number of individual kₘ values selected, either by modeling a MIP with multiple kₘ values, or a collection of MIPs having a single, unique kₘ value, results in reduced RMS error and a closer fit between the desired and anticipated (calculated) profiles for either releasing or adsorbing a selected material. Again, in other embodiments, a collection of individual MIP matrices having unique and significantly different kₘ values could also be employed, in addition to one or a plurality of individual MIP matrices having one or more different kₘ values, and combinations thereof, to achieve an novel MIP system that operates to controllably release and/or adsorb one or more selected materials according to a desired “catching” and/or “release” profile for each selected material.

It is to be noted that in additional embodiments, both the novel method and the novel MIPs can be selected to achieve a MIP matrix and/or MIP system that can operate to catch or release, or both, any selected materials or combination of different materials, following virtually any conceivable desired profile, including desired delays that can be achieved using MIPs exhibiting at least two or more significantly different (unique) average associative binding constants, optionally in combination with a delay release element associated with one or more of the MIPs.

### Complementary Molecular Pairing Examples

In another series of embodiments, the novel MIP polymers, optionally in the form of beads, coatings, particles, fibers, fiber webs, foams, films, sheets and/or combinations thereof, may be used to both simultaneously release a selected first material into a system and to remove a selected second material from that same system. Applications were this method of using the novel MIP polymers and devices constructed thereof include the release of drugs and medicines while removing potentially contra-indicated materials that would otherwise interfere or negate the desired effect of the delivered drug and/or medicine.

For example, theophylline is prescribed for the treatment of Chronic Obstructive Pulmonary Disorder (COPD), a disease that effects a large number of human patients and for which the medicine acts as a bronchodilator to ease breathing. In the illustration below, the structures of theophylline (I) and caffeine (II) are compared, and seen to differ only in caffeine having one additional methyl group on the five-membered indole ring. Other potential compounds that could be employed as TIEs to produce modified associative binding site kinetics include for example, but are not limited to 3-Isobutyl-1-methylxanthine (Structure III) and 3,7-Dimethyl-1-prop argylxanthine (Structure IV).

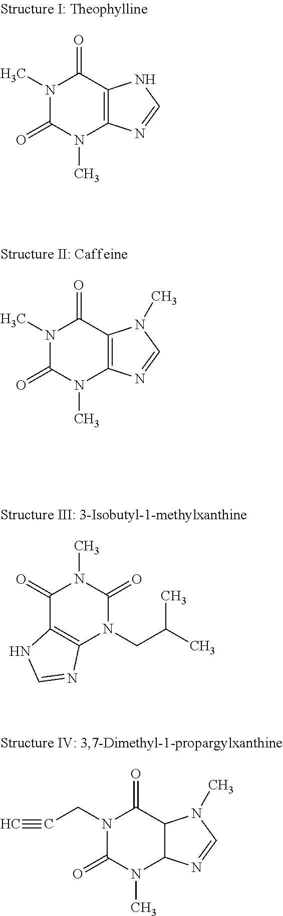

Thus, in one embodiment, the novel MIP polymers are imprinted with caffeine as the selected TIE during polymerization, and the caffeine later extracted from the MIP polymer matrix. Then, theophylline is infused into the resulting caffeine-imprinted polymer matrix, whose MIP sites, owing to the similarity in molecular structures, will act to bind the theophylline, but not irreversibly because the molecules are distinguished by a difference in the molecular structure, and caffeine having been the imprinted entity, will still retain a higher binding efficacy as it is a much closer molecular fit. Accordingly, in this embodiment, the theophylline infused MIP polymers, formulated into a dosage form that can be ingested, such as a tablet or capsule, can be ingested. Once ingested, the theophylline will be released while any free caffeine simultaneously present in the stomach and digestive track, for example, will be strongly and irreversibly adsorbed by the MIP polymers. Further, due to the similar molecular geometries, theophylline is likely to be released slower than if dosed directly, as the MIP binding sites will have some affinity for the molecule, but not as strong a binding efficacy as caffeine, but will act to release the theophylline over time due to diffusion and equilibrium concentration effects accordingly, even if no caffeine is present to displace the infused theophylline.

In yet a further embodiment, the novel MIP polymers are imprinted with caffeine as the selected TIE, and the caffeine extracted from the polymers, and the MIP polymer is then added to or formulated into a dosage form that can be ingested, such as a tablet or capsule also having the requisite amount of theophylline present, optionally in a readily assimilated form or alternatively in a slow release dosage form. Once ingested, the theophylline would be released from the dosage form as it contacts stomach fluids and enters the digestive tract, and the novel MIP polymers would also disperse as well, but due to having caffeine binding sites present on their surfaces, would adsorb caffeine present in the stomach and/or intestinal tract so as to limit or prevent caffeine being absorbed into the bloodstream while the medicine is being absorbed.

Table 6 shows examples of some measured kinetic data that can be used in the design, programming and selection of the novel MIP systems. Kinetic data reveals multiple choices of monomer and co-monomer, TIE material, porogen and use of associative molecules to generate various example MIPs with modified average associative binding constants, here for theophylline. Example 1 represents the results of a standard approach to making a MIP, showing the results of a study by Norell, M. C., et. al., (see footnote 1) revealing a conventional MIP templated using theophylline as the TIE and a methacrylic acid monomer as the polymer formation starting materials, resulting in a MIP with a high dissociation constant (k_{diss}) of about 1.0×10⁻⁵ mM/g-min with respect to theophylline. Note that the constant cited is for dissociation, so that the corresponding association constants are inversely proportion in value (i.e. a smaller dissociation constant correlates to a larger association constant, and vice versa). According to one embodiment method of the present disclosure, shown as Example 2 in Table 6, one would design a MIP with modified average association binding constants by including a material that acts as an “associative molecule” in conjunction with the TIE material during the MIP polymerization process, the associative molecule selected being any compatible material that associates with the TIE material or has multiple similar molecular features unique to the TIE material, which results in the formation of binding sites exhibiting increased average associative binding constants compared to the conventional MIP Example 1.

In Example 2, the resulting MIP would be suitable for a catching system with an improved, or superoptimal average associative binding constant, thus having the potential for improved adsorption and retention of the targeted material to be controllable absorbed.

Example 3 illustrates the use of a co-monomer in the polymer system to modify the binding characteristics of the TIE material. In this example, the more polar vinyl acetate monomer is incorporated into the MIP matrix during polymerization, resulting in the formation of binding sites with a lower average associative binding constant corresponding to sites having lower affinity for the TIE material (here, theophylline) likely owing, without being bound by theory, to the decreased hydrophobicity of resulting binding sites due to the presence of vinyl acetate groups in the resulting MIP matrix. In Example 4, a theophylline-like material, 3-Isobutyl-1-methylxanthine, having some similar structural features to theophylline, but also being a larger, bulkier molecule, is used as a TIE, the MIP being formed using methacrylic acid in a solvent, resulting in a somewhat larger dissociation constant of 2.0×10⁻⁴ mM/g-min, so that with respect to theophylline, the latter would have a somewhat lower average associative binding affinity, such that kₘ<<knE.

In Example 5, another theophylline-like material, 3,7-Dimethyl-1-propargylxanthine is used as the TIE, the MIP being formed using methacrylic acid in a solvent under otherwise identical conditions as Example 4, and resulting in a much larger dissociation constant of 8.0×10⁻⁴ mM/g-min, so that with respect to theophylline, the latter would have a substantially (much) lower average associative binding affinity, such that kₘ<<<km.

In Example 6, theophylline itself is used as the TIE in combination with an associative molecule 1 and the addition of a select porogen 2, in addition to the solvent system, the MIP being formed using methacrylic acid in a solvent under otherwise identical conditions as Example 4. Owing to the use of an associative molecule and a select porogen, the resulting binding sites within the MIP have a much lower dissociation constant of 1.0×10⁻⁶, showing that the additional presence of a second molecule and the choice of porogen can substantially alter the resulting binding characteristics of the MIP matrix even with respect to the actual TIE material used for imprinting. Here, the lower dissociation constant produces a MIP with an average associative binding constant with respect to theophylline that is substantially greater than that achieved in the other example approaches, resulting in km>>k_{TIE}, the k_{TIE} reference value being that of the “unmodified” TIE binding sites formed in MIP Example 1.

TABLE 6
Various MIPs with Modified Average Associative
Binding Constants for Theophylline

| Exam- | | | k_{diss} (3) | |
|---|---|---|---|---|
| ple | Polymer | | (mM/g- | kₘ vs. |
| # | System (1) | Template (2) | min) | k_{TIE} (4) |
| 1 | Methacrylic Acid | Theophylline | 1.0 × 10⁻⁵ | = |
| | Solvent (5) | | | |
| 2 | Methacrylic Acid | Theophylline + | 3.0 × 10⁻⁶ | kₘ > k_{TIE} |
| | Solvent | Associative | | |
| | | Molecule 1 | | |
| 3 | Methacrylic | Theophylline | 1.0 × 10⁻⁴ | kₘ < k_{TIE} |
| | Acid + | | | |
| | Vinyl Acetate | | | |
| | Solvent | | | |
| 4 | Methacrylic Acid | 3-Isobutyl-1- | 2.0 × 10⁻⁴ | kₘ << k_{TIE} |
| | Solvent | methylxanthine | | |
| 5 | Methacrylic Acid | 3,7-Dimethyl-1- | 8.0 × 10⁻⁴ | kₘ <<< k_{TIE} |
| | Solvent | propargylxanthine | | |
| 6 | Methacrylic Acid | Theophylline + | 1.0 × 10⁻⁶ | kₘ >> k_{TIE} |
| | Solvent | Associative | | |
| | | Molecule 1 + | | |
| | | Porogen (6) | | |

(1) Norell, M. C., et. al., “Theophylline Molecularly Imprinted Polymer Dissociation Kinetics”, Jour. Of Molecular Recognition, Vol. 11, 98-102, 1998. An average dissociation value for theophylline of about 1 × 10⁻⁵ is a reasonable starting approximation of the value for a conventional MIP using the same material (theophylline) as the templating entity.
(2) Example template entities and additional associative molecules, choice of porogen (solvent), selected to achieve desired average associative binding constant.
(3) Example M is actual measured value from reference, footnote (1) above. Note that dissociation constants are inversely proportional to their respect association constants.
(4) Estimated kₘ values with respect to modified average associative binding constant as influenced by choice of template(s), porogen, polymer type (monomers), associative molecules, solvent and polymerization conditions employed to produce a MIP.
(5) Standard solvent system used as reported by reference, footnote (1) above.
(6) Alternative solvent or cosolvent added.

In additional embodiments, any drug or medicine having a known molecular or biological contra-indicated agent that can be imprinted (hence being used as a TIE) can be combined in a single dosage form in combination with a medicine, so that the medicine can be ingested and absorbed as needed, while the MIP polymer operates to adsorb the contra-indicated agent so as to prevent the simultaneous adsorption of the undesired agent with the medicine. In some embodiments, the medicine can simply be infused into the MIP polymers that have been imprinted with the contra-indicated TIE, while in other embodiments, the medicine can simply be coformulated or compounded with the MIP polymers into a single dosage form. In further embodiments, the medicine can be associated with the novel MIPs, MIP matrices and MIP systems in order to be delivered in a programmed and time-controlled manner, with or without a delay functionality, in order to achieve any desired dosage profile, while simultaneously being coupled with a second MIP that has been imprinted with a TIE, the TIE being a second contra-indicated material that is to be absorbed from a fluid media while the novel MIPs release the desired medicine into that same fluid media.

Table 7 provides a list of common drugs and medicines and their known contra-indicated agents that interfere with the medicine and/or cause undesirable side effects when both materials are present and/or absorbed simultaneously into the bloodstream during treatment.

In some embodiments, a polymer that would not be degraded or decomposable under physiological conditions found within a target body organ system, such as but not limited to the stomach, intestine, blood stream, lung, tumor, or other organ or bodily fluid, would be preferred so as not to release the adsorbed contra-indicated material while still present in the body.

In other embodiments, a degradable polymer that eventually is degraded, decomposed or metabolized under physiological conditions found within a target body organ system could suitably be employed by selecting a polymer that would resist the degradation while it adsorbs the selected material, but degrades and releases the material back into the system after the primary medicine has had a chance to be absorbed and/or exert its beneficial physiological benefit while the contra-indicated material has been temporarily bound and rendered ineffective in interfering with the medicine for some selected period of time, which can be adjusted by appropriate selection of the polymer material used to form the MIP polymers, and the optional use of a delay functionality associated with one or more of the MIPs or MIP matrices employed.

Accordingly, in one embodiment, caffeine imprinted polymers could be used in any suitable selected dosage form in conjunction with a medicine, including but not limited to albuterol, theophylline, ciprofloxacin, levofloxacin, moxifloxacin, linezolid, aripiprazole, clozapine, olanzapine, quetiapine, risperidone, and ziprasidone, and combinations thereof.

In another embodiment, MIP polymers imprinted with extracts of glycyrrhizin, St. John's Wort and/or Senna could be used in any suitable selected dosage form in conjunction with a medicine, including but not limited to digoxin and glycoside-based medicants, and combinations thereof.

In yet further embodiments, MIP polymers imprinted with tyramine and/or histamine could be used in any suitable selected dosage form in conjunction with a medicine, including but not limited to oxazolidinone, oxazolidinon-derived antibacterials, linezolid, anti-mycobacterial, ethambutol, isoniazid, rifampin, combinations of rifampin and isoniazid, combinations of rifampin, isoniazid and pyrazinamide, monoamine oxidase inhibitors, phenelzine, tranylcypromine, and combinations thereof.

In other embodiments, MIP polymers imprinted with warfarin could be used in any suitable selected dosage form in conjunction with a medicine, including but not limited to statins, atorvastatin, fluvastatin, lovastatin, pravastatin, simvastatin, rosuvastatin, gemfibrozil, and combinations thereof.

In yet another embodiment, MIP polymers imprinted with Vitamin K could be used in any suitable selected dosage form in conjunction with a medicine, including but not limited to anticoagulants, warfarin, and the like.

In a further series of embodiments, MIP polymers imprinted with a selected NSAID (non-steroidal anti-inflammatory drug), such as but not limited to acetylsalicylic acid (aspirin), celecoxib (Celebrex™), dexdetoprofen (Keral™), diclofenac (Voltaren™, Cataflam™, Voltaren-XR™), diflunisal (Dolobid™), etodolac (Lodine™, Lodine™ XL), etoricoxib (Algix™), fenoprofen (Fenopron™, Nalfron™), firocoxib (Equioxx™, Previcox™), flurbiprofen (Urbifen™, Ansaid™, Flurwood™, Froben™), ibuprofen (Advil™, Brufen™, Motrin™, Nurofen™, Medipren™, Nuprin™), indomethacin (Indocin™, Indocin™ SR), ketoprofen (Actron™, Orudis™, Oruvail™, Ketoflam™), ketorolac (Toradol™, Sprix™), licofelone, lornoxicam (Xefo™), loxoprofen (Loxonin™, Loxomac™, Oxeno™), lumiracoxib (Prexige™), meclofenamic acid (Meclomen™), mefenamic acid (Ponstel™), meloxicam (Movalis™, Melox™, Recoxa™, Mobic™), nabumetone (Relafen™), naproxen (Aleve™, Anaprox™, Midol™, Naprosyn™, Naprelan™), nimesulide (Sulide™, Nimalox™, Mesulid™), oxaporozin (Daypro™, Dayrun™, Duraprox™), parecoxib (Dynastat™), piroxicam (Feldene™), rofecoxib (Vioxx™, Ceoxx™, Ceeoxx™), salsalate (Mono-Gesic™, Salflex™, Disalcid™, Salsitab™), sulindac (Clinoril™), tenoxicam (Mobiflex™), tolfenamic acid (Clotam™ Rapid, Tufnil™), and/or valdecoxib (Bextra™) could be used in any suitable selected dosage form in conjunction with a medicine, including but not limited to intracellular proton pump inhibitors, dexlansoprazole, esomeprazole, lansoprazole, omeprazole, pantoprazole, rabeprazole, and combinations thereof.

In another embodiment, MIP polymers imprinted with a potassium ion binding entity could be used in any suitable selected dosage form in conjunction with a medicine, including but not limited to ACE (angiotensin converting enzyme) inhibitors, captopril, enalapril, lisinopril, moexipril, quinapril, ramipril, diuretics, bumetanide, furosemide, hydrochloro-thiazide, metolazone, triamterene, triamterene combined with hydrochlorothiazide, and combinations thereof. The above illustration provides many different embodiments or embodiments for implementing different features of the disclosure. Specific embodiments of components and processes are described to help clarify the disclosure. These are, of course, merely embodiments and are not intended to limit the disclosure from that described in the claims.

TABLE 7
Contra-Indicated Drug Interactions (1)

| Disease/Medical | Indicated | Contra- | |
|---|---|---|---|
| Condition | Medication | indicated Agent | Adverse Effect |
| Asthma | Bronchodilators: | caffeine | Using |
| Bronchodilators treat | albuterol | | bronchodilators |
| and prevent | theophylline* | | with foods and |
| breathing problems | | | drinks that have |
| from bronchial | | | caffeine can |
| asthma, chronic | | | increase the |
| bronchitis, | | | chance of side |
| emphysema, and | | | effects, such as |
| chronic obstructive | | | excitability, |
| pulmonary disease | | | nervousness, and |
| (COPD). These | | | rapid heart beat |
| medicines relax and | | | |
| open the air | | | |
| passages to the | | | |
| lungs to relieve | | | |
| wheezing, shortness | | | |
| of breath | | | |
| Antibacterials | Quinolone | caffeine | Use of |
| Medicines known as | Antibacterials: | | ciprofloxacin may |
| antibiotics or | ciprofloxacin | | result in the |
| antibacterials are | levofloxacin | | buildup of |
| used to treat | moxifloxacin | | caffeine in the |
| infections caused by | | | body |
| bacteria | | | |
| Blood Pressure | ACE Inhibitors: | potassium | ACE inhibitors |
| Regulators: ACE | captopril | | can increase the |
| (Angiotensin | enalapril | | amount of |
| Converting Enzyme) | lisinopril | | potassium in |
| inhibitors alone or | moexipril | | your body |
| with other medicines | quinapril | | (hyperkalemia). |
| lower blood pressure | ramipril | | Too much |
| or treat heart failure. | | | potassium can be |
| They relax blood | | | harmful and can |
| vessels so blood | | | cause an |
| flows more smoothly | | | irregular |
| and the heart can | | | heartbeat and |
| pump blood better | | | heart palpitations |
| | | | (rapid |
| | | | heartbeats). |
| Diuretics for control | Diuretics: | potassium | Diuretics, like |
| of Blood Pressure | bumetanide | | triamterene (not |
| and Fluid Retention | furosemide | | with |
| | hydrochloro- | | hydrochlorothiazide), |
| | thiazide | | lower the |
| | metolazone | | kidneys' ability to |
| | triamterene | | remove |
| | (triamterene + | | potassium, which |
| | hydro- | | can cause high |
| | chlorothiazide) | | levels of |
| | | | potassium in the |
| | | | blood stream |
| | | | (hyperkalemia). |
| | | | Too much |
| | | | potassium can be |
| | | | harmful and can |
| | | | cause an |
| | | | irregular or rapid |
| | | | beating of the |
| | | | heart. |
| Glycosides treat | Glycosides: | glycyrrhizin | Digoxin with |
| heart failure and | digoxin | St. John's Wort | glycyrrhizin can |
| abnormal heart | | Senna | cause irregular |
| rhythms. They help | | | heart beat and |
| control the heart | | | heart attack. |
| | | | Avoid taking |
| | | | digoxin with |
| | | | Senna and St. |
| | | | John's Wort since |
| | | | they may |
| | | | decrease the |
| | | | amount and |
| | | | action of digoxin |
| | | | in your body. |
| Lipid-Altering Agents | Statins: | Grapefruit juice | Large amounts of |
| (also called Statins) | atorvastatin | warfarin | grapefruit juice |
| or | fluvastatin | | can raise the |
| (HMG-CoA | lovastatin | | levels of statins in |
| reductase inhibitors) | pravastatin | | your body and |
| Statins lower | simvastatin | | increase the |
| cholesterol by | rosuvastatin | | chance of side |
| lowering the rate of | gemfibrozil | | effects if taking |
| production of LDL | | | atorvastatin, |
| (low-density | | | lovastatin, or |
| lipoproteins, or | | | simvastatin. |
| sometimes called | | | Combining |
| “bad cholesterol”). | | | gemfibrozil and a |
| | | | statin increases |
| | | | risk of |
| | | | rhabdomyolysis |
| | | | and subsequently |
| | | | renal failure |
| Vitamin K Agonists/ | warfarin | Vitamin K | Vitamin K in food |
| Anticoagulants | | | can make the |
| Anticoagulants are | | | medicine less |
| also called “blood | | | effective. |
| thinners.” They lower | | | |
| the chance of blood | | | |
| clots forming or | | | |
| growing larger in | | | |
| your blood or blood | | | |
| vessels. | | | |
| Gastroesophageal | Proton Pump | NSAID | Treatment to |
| Reflux Disease | Inhibitors: | Non-steroidal | reduce the risk of |
| (GERD) and Ulcers | dexlansoprazole | anti- | stomach ulcers in |
| Proton Pump | esomeprazole | inflammatory | people taking |
| Inhibitors (PPIs) | lansoprazole | drugs: | nonsteroidal anti- |
| work by decreasing | omeprazole | ibuprofen | inflammatory |
| the amount of acid | pantoprazole | | drugs (NSAIDs) |
| made in the | rabeprazole | | |
| stomach. They treat | | | |
| conditions when the | | | |
| stomach produces | | | |
| too much acid. | | | |
| Antibacterials | Oxazolidinone | tyramine | High levels of |
| | Antibacterials: | caffeine | tyramine can |
| | linezolid | | cause a sudden, |
| | | | dangerous |
| | | | increase in blood |
| | | | pressure. |
| Antimycobacterials | Anti- | tyramine | High levels of |
| treat infections | mycobacterials: | histamine | tyramine can |
| caused by | ethambutol | | cause a sudden, |
| mycobacteria, a type | isoniazid | | dangerous |
| of bacteria that | rifampin | | increase in your |
| causes tuberculosis | rifampin + | | blood pressure. |
| (TB), and other kinds | isoniazid | | Foods with |
| of infections. | (rifampin + | | histamine |
| | isoniazid + | | can cause |
| | pyrazinamide) | | headache, |
| | | | sweating, |
| | | | palpitations |
| | | | (rapid |
| | | | heartbeats), |
| | | | flushing, and |
| | | | hypotension (low |
| | | | blood pressure). |
| Antidepressants- | MAOI: | tyramine | High levels of |
| Monoamine Oxidase | phenelzine | | tyramine can |
| Inhibitors (MAOIs) | tranylcypromine | | cause a sudden, |
| MAOIs treat | | | dangerous |
| depression in people | | | increase in your |
| who haven't been | | | blood pressure. |
| helped by other | | | |
| medicines. | | | |
| Antipsychotics treat | Antipsychotics: | caffeine | Avoid caffeine |
| the symptoms of | aripiprazole | | when using |
| schizophrenia and | clozapine | | clozapine because |
| acute manic or | olanzapine | | caffeine can |
| mixed episodes from | quetiapine | | increase the |
| bipolar disorder. | risperidone | | amount of |
| | ziprasidone | | medicine in your |
| | | | blood and cause |
| | | | side effects. |

(1) Title: Avoid Food Drug Interactions, Published by: National Consumers League and the US FDA, Source: U.S. Department of Health and Human Services - FDA Division, Online: www.nclnet.org or www.fda.gov/drugs, Publication Number: (FDA) CDER 10-1933.

While the above embodiments relate to drugs and medicines, the novel MIP polymers could also be used to treat other liquids where it is desired to remove a TIE material or TIE-like first material and substitute and/or release a second material into the treated liquid.

For example, in a series of embodiments, a MIP polymer device in the form of a fiber web fashioned into the form of a spoon or stirring stick, for example, is imprinted with sucrose (sugar), glucose and/or fructose as the TIE material. After extraction of the TIE to produce the imprinted polymer, the MIP polymer web is then dosed with an appropriate level of a desired sweetening agent, including for example, but not limited to sorbitol, mannitol, glycerol, acesulfame potassium, aspartame, cyclamate, isomalt, saccharin, sucralose, alitame, thaumatin, neohesperidine dihydrochalcone, aspartame-acesulfame salt, maltitol, lactitol, xylitol, stevia, and erythritol, or combinations thereof, which are released into the treated liquid, effectively replacing the original sugars with an artificial sweetener and effectively turning any sugar-containing beverage into a lower calorie sugar-free dietary beverage.

In another embodiment, a MIP polymer device in the form of a fiber web fashioned into the form of a spoon or stirring stick, for example, is imprinted with a sodium ion binding entity. After extraction of the TIE (here a sodium salt to maintain ionic neutrality) to produce the imprinted polymer, the MIP polymer web is then dosed with an appropriate level of a salt substitute, for example but not limited to a potassium salt of chloride, bromide, nitrate, sulfate, hydroxide, and/or combinations thereof, which are released into the treated liquid, effectively replacing the original sodium with a healthier substitute and rendering the liquid sodium free or at least reducing the sodium level substantially. In one embodiment, two MIP matrices with identical magnitudes of association and dissociation constants are also within the scope of the present disclosure and can be useful. For example, a combination of novel MIP matrices (separate MIP polymers) could be combined in a MIP system with either some separation in space (sharing contact with the same fluid media, but spatially apart by at least some effective distance relative to the system being treated, i.e. with some intermediary shared volume of fluid media that is desired to be treated) or separation in time (one or more of the MIP polymers or MIP matrices coupled with a time-release coating so as to delay its contact with a shared fluid media) having the same magnitude of associative and dissociative rates, or averages thereof, so that the first “release” MIP matrix controllable releases its payload material, while after some (optionally extended) delay at least in part dictated by the rate of the material's diffusion into and throughout the volume of shared fluid media, the second “catch” MIP matrix controllable absorbs free material at the same rate, thus enabling a transitory release of the payload material into the shared volume of fluid media or vicinity of the dual MIP system, the material then being scavenged by the second MIP matrix. In further embodiments, selection of the overall binding capacity of the second MIP matrix could be adjusted to leave a net amount of unabsorbed (owing to the second MIP matrix material binding capacity being lower than that of the first MIP matrix initially holding and releasing the material payload) material in the shared fluid media.

### Open and Closed Media Systems

In embodiments in which the novel MIP matrices and systems described herein are used in closed media systems, i.e., wherein the MIP polymer and fluid media are of finite volume and no other addition, exchange or loss of the target select material of interest occurs, the closed system is expected to eventually achieve an equilibrium condition, wherein the amount of material present associated with the novel MIPs and the amount of material present in the fluid media have reached their steady state concentrations as dictated by the relative forward binding and reverse release rate ratio, or equilibrium constant. However, for the initial period and nearly most of the period of time prior to the system achieving equilibrium, the initial rate(s) of release or adsorption of a material (km) still dominate the respective release or catching mechanisms, enabling these kinetic rate(s) to be used to reasonably approximate the controlled release and catch profiles of the novel MIP system.

In embodiments in which the novel MIP matrices and systems described herein are use in open systems, i.e., wherein the MIP polymer and fluid media are not necessarily fixed in space or volume, such as for example but not limited to changing volumes or dynamic (flowing or exchanging) fluid systems wherein the selected material is being consumed or dispersed into the space or volume so that static equilibrium conditions are not expected to prevail, then the dynamic forward release or reverse adsorption (catching) kinetics are expected to adequately describe and enable prediction of the novel MIP system's controlled release or controlled adsorption profiles, respectively, to an acceptable degree of accuracy.

### Example MIP Polymer Forms

The novel MIP polymers, matrices and systems may be formed in a variety of physical forms and configurations. Table 8 below illustrates some example embodiments, and non-limiting examples, of various MIP polymer forms and potential application areas where such disclosed forms could be used or applied for either adsorbing or releasing materials into a fluid media.

TABLE 8
Various MIP Polymer Forms and Application Areas

| Form | Potential Utility or Application Area |
|---|---|
| Particles, | Ingestables (pharmaceuticals by ingestion or |
| Powders, | nanoparticles via injection) |
| Granules | Packed column beds or devices (contained but |
| | permeable) |
| | Incorporated into coatings (paints, finishes) |
| | Incorporated into other water or solvent permeable |
| | materials |
| | Particulate products (fertilizers, spill kits, |
| | additives to products) |
| | Agglomerated products (cat litter, absorbents, |
| | fertilizers) |
| Fibers | Ingestables |
| | Incorporated into fabrics, polymers (water or |
| | solvent permeable materials) |
| | Analytical and scientific measuring and diagnostic |
| | devices |
| | Monitoring and metering systems |
| | Fiber products (sutures, dental floss) |
| | Bicomponent fibers; functionality outside, low- |
| | cost structure inside |
| Fiber Webs, | Textiles - bedding, clothing |
| Woven and | Medical fabrics (bandages, wraps, clothing, |
| Non-wovens, | masks, gowns, sponges) |
| Sponges | Filter media (coffee filters, air filters, water filters, |
| | HEPA devices) |
| | Shaped fiber objects (compressed plugs, fittings, |
| | septums, stoppers, etc.) |
| Films, Membranes | Coatings, cast films on surfaces (countertops, |
| | tools, devices) |
| | Films formed by in situ polymerization onto |
| | surface of object or mold (condoms, catheters, |
| | medical inserts, stents, subdural and subdermal |
| | implants, devices like insulin pumps, hearing |
| | devices, vision aids, heart pacers and the like, |
| | inside coatings of packaging, cans, bottles, etc.) |
| | Self-supporting films (sheets, wraps, packaging |
| | materials) |
| | Formed onto supporting materials (permeable, |
| | porous, so potentially dual-active MIP surface) |
| Laminates | Films formed and applied to surfaces |
| | (antimicrobial cutting boards, medical devices, |
| | infection control on objects) |
| | Applied to supporting materials (impermeable, |
| | non-porous substrate so only one-active MIP |
| | surface) |
| Cast Objects | In-mold polymerization to form shaped objects |
| | (inserts, plugs, mechanical parts of devices, |
| | contact lenses) |
| | Any cast shape or object currently formed by |
| | plastics (pipes, utensils, tools, insulation, etc.) |
| Gel Matrices | Ingestables (release drugs at controlled rate then |
| (Controlled | dissolve) |
| solubility MIPs) | Water-treatment |
| | Food prep/food storage (temporarily capture |
| | undesired material) |
| | Cleaners |
| Ionic Liquids | Smart Liquids and Solids |
| (MIPs with | Lubricants with catch and/or release functionality |
| Ionic Liquid | Foams as fire retardants etc |
| Salts) | Energetic Smart Materials - Batteries and |
| | Photovoltaic's |

### Example Media

Suitable media in which the MIPs and related MIP matrices and systems of the present disclosure can operate and be used for the delivery or extraction of a selected material include liquids, gases and fluids of human or animal origin, including but not limited to blood, plasma, lymphatic fluid, mucus, saliva, gastric juices, cerebrospinal fluid, sweat, tears, aqueous and vitreous humors of the eye, semen, urine and vaginal secretions, and the like. In general, any media that enables the transport (adsorption and de-adsorption) of a selected material with, into or out of an novel MIP polymer, matrix or associated system, is suitable for use and is included in the scope of the present disclosure.

Additional media include mechanical fluids, such as for example, but not limited to aviation oils and lubricants, axle and transmission oils, bearing and circulating oils, car engine oils, compressor oils, electrical oils, gear oils, greases, diesel engine oils, hydraulic fluids, marine lubricants, process oils, slideway oils and turbine oils, and the like.

Also included are cooling system fluids, such as for example, but not limited to engine coolants, antifreeze, fuel coolants, hydraulic oils, corrosion inhibitors, engine oil coolers, and the like.

Additional media include refrigerants, such as for example, but not limited to CFC (chlorofluorocarbons), CFO (chlorofluoroolefins), HCFC (hydrochlorofluorocarbons), HCFO (hydrochlorofluoroolefins), HFC (hydrofluorocarbons), HFO (hydrofluoroolefins), HCC (hydrochlorocarbons), HCO (hydrochloroolefins), HC (hydrocarbons), HO (hydroolefins and alkenes), PFC (perfluorocarbons), PFO (perfluoroolefins), PCC (perchlorocarbons), PCO (perchloroolefins) and H (halons and haloalkanes), and the like.

Additional media include herbicidal fluids and related carrier solvents, such as for example, but not limited to those materials applied to the ground, seeds, sprouts, plants, plant debris, flowers, fruit, vegetables, roots, leaves, buds, bark, and the like, including acaricides, antifungals, antimicrobials, bacteriosides, bacteriostats, disinfectants, germicides, nematacides, and the like.

Additional media include alcoholic based beverages, such as for example, but not limited to ale, beer, cauim, chicha, cider, desi daru, haungjiu, icariine liquor, kilju, kumis, mead, nihamanchi, palm wine, pulque, sake, sonti, tepache, tonto, tiswin, wine and other alcoholic liquids, including ferments, condensates, distils and extracts, and the like.

Suitable media include non-alcoholic beverages and foods, such as for example, but not limited to water, milk and dairy-based beverages, soy-based and nut-based beverages, juices, vegetable extracts and juices, coffee, tea, soft drinks, carbonated beverages, sports beverages, energy drinks, and the like.

Additional media include vegetable oils, such as for example, but not limited to major oils (Coconut oil, Corn oil, Cottonseed oil, Olive oil, Palm oil, Peanut oil, Rapeseed oil, Canola oil, Safflower oil, Sesame oil, Soybean oil, Sunflower oil and the like), nut oils (Almond oil, Beech nut oil, Brazil nut oil, Cashew oil, Hazelnut oil, Macademia oil, Mongongo nut oil, Pecan oil, Pine nut oil, Pistachio oil, Walnut oil and the like), citrus oils (Grapefruit seed oil, lemon oil, orange oil and the like), melon and gourd oils (Bitter gourd oil, bottle gourd oil, buffalo gourd oil, butternut squash seed oil, Egusi seed oil, Pumpkin seed oil, watermelon seed oil, and the like), food supplement oils (Acai oil, Black seed oil, Black currant seed oil, Borage seed oil, Evening primrose oil, Flaxseed oil and th like) and other edible oils (amaranth oil, apricot oil, apple seed oil, Argan oil, Avocado oil, Babassu oil, Ben oil, Tallow nut oil, Chestnut oil, Carob pod oil, Cocoa butter, Cocklebur oil, Cohune oil, coriander seed oil, date seed oil, Dika oil, False flax oil, Grape seed oil, Hemp oil, Kapok seed oil, Kenaf seed oil, Lallemantia oil, Mafura oil, Marula oil, Meadowfoam seed oil, Mustard oil, Niger seed oil, Poppy seed oil, Nutmeg butter, Okra seed oil, Papaya seed oil, Perilla seed oil, Persimmon seed oil, Pequi oil, Pili nut oil, Pomegranate seed oil, Poppyseed oil, Pracixi oil, Prune kernel oil, Quinoa oil, Ramtil oil, Rice bran oil, Royle oil, Shea nut oil/butter, Sacha inchi oil, Sapote oil, Seje oil, Taramira oil, Tea seed oil, Thistle oil, Tigernut oil, Tobacco seed oil, Tomato seed oil, Wheat germ oil), and the like.

Suitable media include vinegars, such as for example, but not limited to apple cider, Balsamic, beer, cane, coconut, Date, distilled, fruit, honey, malt, Palm, raisin, rice, sherry, spirit, white and wine vinegars, and the like.

Additional media include sauces and condiments, such as for example, but not limited to brown sauces (Bordelaise, chateaubriand, charcutiere, demi glace, gravy, poutine, romesco, sauce africane, sauce au poivre, wine), butter sauces (beurre maine, café de paris, meuniere sauce), emulsified sauces (aioli, béarnaise sauce, hollandaise sauce, mayonnaise, remoulade, salad crème, tartar sauce), green sauces (salsa verde), hot sauces (Phrik nam pla, buffalo sauce, chili sauce, datil pepper sauce, enchilada sauce, tabasco sauce), meat-based sauces (amatriciana, barese ragu, Bolognese, carbonara, Cincinnati chile, Neapolitan ragu, picadillo, ragu, sloppy joe), sauces from fresh, chopped ingredients (chimichurri, gremolota, muidei, onion sauce, persillade, pesto, pico de gallo, salsa cruda, salsa verde, sauce gribiche, sauce yierge, tkemali), sweet sauces (butterscotch sauce, caramel sauce, chocolate gravy/sauce, custard/crème anglaise, fudge sauce, fruit sauces), white sauces (béchamel sauce, mushroom sauce, Mornay sauce, sauce Allemande, sauce Americaine, supreme sauce, yogurt sauce), and the like.

Additional media include liquid effluent and process streams, such as for example, but not limited to waste water, blackwater (human waste), cesspit, septic, sewage, rain water, groundwater, surplus manufactured liquids from domestic urban rainfall runoff, seawater ingress, direct ingress of river water, direct ingress of manmade liquids, spills, highway drainage, storm drain runoff, industrial waste streams, industrial site drainage, industrial process waters, organic waste, organic or non bio-degradable/difficult-to-treat waste streams, toxic waste, emulsions, agricultural drainage, hydraulic fracturing, and the like.

Suitable media include fluids that are liquid at elevated temperatures and/or pressures, such as for example, but not limited to molten solids, liquid metals and composite, supercritical liquids, and the like.

Additional media include gaseous fluids, such as for example, but not limited to gas effluent streams from stationary sources including smoke stacks of power plants, manufacturing facilities (factories) and waste incinerators, as well as furnaces and other types of fuel-burning heating devices, mobile sources including motor vehicles, marine vessels, and aircraft, controlled burn practices in agriculture and forest management, fumes from paint, hair spray, varnish, aerosols and other solvents, waste deposition in landfills, military resources, such as nuclear weapons, toxic gases, germ warfare, and rocketry, air borne dust streams, and the like.

Additional media include gases, such as for example, but not limited to elemental (atomic) gases, gaseous compounds, molecular gases, air and other mixed gases, liquid-saturated gases and partially saturated gases, fumes, smoke (gas, plus entrained solids), tobacco smoke, pipe smoke and fireplace smoke, and the like.

### APPENDIX

This disclosure is accompanied by an Appendix which includes copies of all the equations. The Appendix is included by reference as if fully set forth herein.

Although the disclosure is illustrated and described herein as embodied in one or more specific examples, it is nevertheless not intended to be limited to the details shown, since various modifications and structural changes may be made therein without departing from the spirit of the disclosure and within the scope and range of equivalents of the claims. Accordingly, it is appropriate that the appended claims be construed broadly and in a manner consistent with the scope of the disclosure, as set forth in the attached claims.

What is claimed is:

1. A polymeric matrix comprising:
a plurality of binding sites within a molecularly imprinted polymer (MIP) that exhibit at least one average associative binding constant (kₘ) with respect to a selected material (m);
wherein the magnitude of said average associative binding constant is significantly different than that of the average equilibrium associative binding constant exhibited by said polymer matrix for a reference target imprinted entity (TIE) used as the template forming entity in the formation of said plurality of binding sites within said MIP; and
wherein said plurality of binding sites operate to enable the controlled capture and the controlled release of said selected material, and combinations thereof, when in contact with a fluid media.

2. The polymeric matrix of claim 1, wherein said molecularly imprinted polymer is formed by means of polymerizing a plurality of monomers into a three dimensional matrix in the presence of a target imprinted entity, a porogen, optionally a cosolvent, optionally comonomers, optionally a pore modifying agent, and optionally a cross-linking agent, and combinations thereof.

3. The polymeric matrix of claim 1, wherein said molecularly imprinted polymer with said plurality of binding sites exhibits at least one average associative binding constant (kₘ) that is suboptimal with respect to a selected material (m) compared to the average associative binding constant (k_{TIE}) of said polymer matrix for a target imprinted entity (TIE) used as the template forming entity in the formation of said plurality of binding sites within said molecularly imprinted polymer.

4. The polymeric matrix of claim 1, wherein said molecularly imprinted polymer comprises two or more sets of binding sites; wherein each said set of binding sites exhibits a significantly different average associative binding constant (kmₙ, n=1,2,3 . . . ) with respect to said selected material;
wherein at least two of said sets (n) of binding sites are formed during a polymerization process using at least one second polymerization aid than is different than a first polymerization aid employed in the formation of a first set of binding sites;
wherein said second polymerization aid is selected from a different TIE, a different porogen, a different solvent, a different cosolvent, a different pore modifying agent, or combinations thereof; and
wherein said significantly different average associative binding constants differ by at least on least significant difference (LSD) unit at the 80% confidence level.

5. The polymeric matrix of claim 4, wherein said sets of binding sites exhibit an average associative binding constant that is significantly lower than that of the average equilibrium associative binding constant exhibited by said polymer matrix for a target imprinted entity (TIE) used as the template forming entity in the formation of said plurality of binding sites within said MIP;
wherein each of said average equilibrium associative binding constants for each of said sets of binding constants are each significantly different in magnitude from each other; and
wherein said average equilibrium associative binding constants differ by at least on least significant difference (LSD) unit at the 80% confidence level, or alternatively at the 90% confidence level, or alternatively at the 95% confidence level, or alternatively at the 99% confidence level.

6. The polymeric matrix of claim 1, wherein said fluid media comprises a fluid selected from air, an aqueous solution, a bodily fluid, a liquid, a chemical composition, a solvent, a vapor, water, and combinations thereof.

7. The polymeric matrix of claim 1, wherein said selected material differs from said target imprinted entity in at least one feature selected from a chemical, physical or stereo isometric characteristic of said target imprinted entity.

8. The polymeric matrix of claim 2, wherein said selected material shares at least one common attribute with said target imprinted entity;
wherein said at least one common attribute is selected from an atom, a chemical group, a chemical bond, a substituent group, an atomic arrangement, a molecular arrangement, a chemical structure, a charge bearing chemical group, an isomer, a stereo-isomer, a sequence of atomic or molecular entities, a three-dimensional structure or portion of a three-dimensional structure, and combinations thereof.

9. The polymeric matrix of claim 1 further comprising a delay element associated with at least one of said molecularly imprinted polymer.

10. The polymeric matrix of claim 1 comprising a combination of two or more distinct molecularly imprinted polymer matrices each having at least one or a plurality of sets of binding sites wherein each said set of binding sites exhibits an average associative binding constant (kmₙ) with respect to said selected material; wherein each of said sets (n) of binding sites is formed during a polymerization process using one of a different monomer, a different comonomer, a different polymer, a different cross-linking agent, a different TIE, a different porogen, a different solvent, a different cosolvent, a different pore modifying agent, or combinations thereof.

11. The polymer matrix of claim 10 further comprising one or a plurality of distinct delay elements each associated with one or more of said distinct molecularly imprinted polymer matrices each having a time delay factor or dissolution characteristic that is significantly different from each other of said other time delay factors or dissolution characteristics.

12. The polymer matrix of claim 1 wherein said at least one average associative binding constant (kₘ) has a value that is less than the average associative binding constant for the TIE used to template said molecular imprinted polymer by one least significant difference (LSD) unit at an 80% confidence level, or alternatively at the 90% confidence level, or alternatively at the 95% confidence level, or alternatively at the 99% confidence level.

13. The polymer matrix of claim 4 wherein said set of average associative binding constants each have values that are less than the average associative binding constant for the TIE used to template said molecular imprinted polymer, and wherein each of said plurality of average associative binding constants for said material are significantly different from each other by at least one significant difference (LSD) unit at an 80% confidence level, or alternatively at the 90% confidence level, or alternatively at the 95% confidence level, or alternatively at the 99% confidence level.

14. The polymer matrix of claim 4, wherein said set of average associative binding constants each have values that are less than the average associative binding constant for the TIE used to template said molecular imprinted polymer; wherein each of said plurality of average associative binding constants for said material differ by at least a factor of two in magnitude with respect to each other.

15. The polymer matrix of claim 4, wherein said set of average associative binding constants each have values that are less than the average associative binding constant for the TIE used to template said molecular imprinted polymer; wherein at least two of said plurality of average associative binding constants for said material differ by at least a factor of two in magnitude with respect to each other.

16. The polymer matrix of claim 4, wherein said set of average associative binding constants each have values that are significantly less than the average associative binding constant for the TIE used to template said molecular imprinted polymer; wherein at least two of said plurality of average associative binding constants for said material differ by at least a factor of ten in magnitude from each other.

17. A molecularly imprinted polymer comprising: a polymeric matrix formed in the presence of a target imprintable entity, a plurality of monomers, a solvent, optionally one or more porogens, and optionally a second plurality of comonomers; wherein said polymeric matrix exhibits at least one set of suboptimal binding sites with an average associative binding constant for a reference material that is lower in magnitude with respect to the average associative binding constant exhibited by the target imprintable entity employed; wherein said reference material is selected from the group consisting of said target imprintable entity, an analog, isomer or derivative of said target imprintable entity, an associative molecule, and combinations thereof.

18. A molecularly imprinted polymer according to claim 17 further comprising at least one porogen, a solvent, and optionally additional comonomers, copolymers, cross-linking agents, coupling agents, and combinations thereof; wherein said polymeric matrix exhibits a plurality of suboptimal binding sites with an average associative binding constant for a reference material that is lower in magnitude with respect to the average associative binding constant exhibited by the target imprintable entity employed; wherein said reference material is selected from the group consisting of said target imprintable entity, an analog, isomer or derivative of said target imprintable entity, an associative molecule, and combinations thereof.

19. A method of controlling the concentration of a material within a fluid media comprising the use of a polymeric matrix comprising:
a plurality of binding sites within a molecularly imprinted polymer (MIP) that exhibit at least one average associative binding constant (kₘ) with respect to a selected material (m);
wherein the magnitude of said average associative binding constant is significantly different than that of the average equilibrium associative binding constant exhibited by said polymer matrix for a reference target imprinted entity (TIE) used as the template forming entity in the formation of said plurality of binding sites within said MIP;
and wherein said plurality of binding sites operate to enable the controlled capture and the controlled release of said selected material, and combinations thereof, when in contact with a fluid media.

20. The method according to claim 19 wherein said polymeric matrix comprises two or more sets of binding sites; wherein each said set of binding sites exhibits a significantly different average associative binding constant (kmₙ, n=1,2,3 . . . ) with respect to said selected material;
wherein at least two of said sets (n) of binding sites are formed during a polymerization process using at least one second polymerization aid than is different than a first polymerization aid employed in the formation of a first set of binding sites;
wherein said second polymerization aid is selected from a different TIE, a different porogen, a different solvent, a different cosolvent, a different pore modifying agent, or combinations thereof;
wherein said significantly different average associative binding constants differ by at least on least significant difference (LSD) unit at the 80% confidence level; and
optionally, a second polymer matrix; wherein said second polymer matrix comprises one or a plurality of distinct delay elements each associated with a first or second molecularly imprinted polymer; wherein said delay element is selected from one or more release coatings, said release coatings having a time delay factor or dissolution characteristic that is significantly different from each other of said other time delay factors or dissolution characteristics.
